(19) United States
(12) Patent Application Publication
(10) Pub. No.: US 2005/0090663 A1
(43) Pub. Date: Apr. 28, 2005

(54) Steroid spirolactonization

(75) Inventors: Thaddeus S. Franczyk II, Chesterfield, MO (US); Grace M. Wagner, St. Louis, MO (US); Bruce A. Pearlman, Kalamazoo, MI (US); Amphlett G. Padilla, Portage, MI (US); Jeffrey L. Havens, Mattawan, MI (US); Sonja S. Mackey, Saint Paul, MN (US); Haifeng Wu, Portage, MI (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia Corporation, St. Louis, MO (US)

(21) Appl. No.: 10/806,081

(22) Filed: Mar. 22, 2004

Related U.S. Application Data
(60) Provisional application No. 60/456,716, filed on Mar. 21, 2003.

(51) Int. Cl. C07J 71/00; C07J 9/00

(52) U.S. Cl. 540/63; 552/548

(57) ABSTRACT

A steroid comprising a 17-spirolactone or corresponding open lactone structure is obtained by carbonylation of a 17-alkenyl or 17-alkynyl substrate. A 17-alkenyl intermediate may be prepared by semi-hydrogenation of a 17-alkynyl group. Multiple reaction schemes are disclosed for preparation of a 3-keto-9,11-epoxy-17-spirolactone steroid such as eplerenone. Novel intermediates are also disclosed, as well as steps for forming such novel intermediates, or converting them to further intermediates or products, by semi-hydrogenation, carbonylation, 6,7-dehydrogenation, furylation or other transformations or combinations thereof.

### CROSS-REFERENCE TO RELATED APPLICATIONS

This application claims priority from application Ser. No. 60/456,716, filed Mar. 21, 2003, the contents of which are hereby incorporated by reference in their entirety.

### BACKGROUND OF THE INVENTION

This invention generally relates to processes for preparing steroid compounds, and more particularly, to processes for preparing steroid compounds having a spirolactone moiety at the C-17 position. In certain preferred embodiments, the invention relates to novel processes for the C-17 spirolactonization of steroid compounds, and novel intermediates produced therein, which are useful in the preparation of methyl hydrogen 9(11)α-epoxy-17α-hydroxy-3-oxopregn-4-ene-7α,21-dicarboxylate, γ-lactone (otherwise referred to as eplerenone or epoxymexrenone).

Methods for preparing 9(11)-epoxy steroids, and eplerenone in particular, are described in co-assigned U.S. patent application Ser. No. 10/392,833, entitled “Processes To Prepare Eplerenone”, filed on even date herewith and hereby incorporated herein by reference in its entirety. Further, methods for preparing C-17 spirolactone steroid compounds are also described in co-assigned U.S. patent application Ser. No. 10/392,857, filed on even date herewith and hereby incorporated herein by reference in its entirety.

### SUMMARY OF THE INVENTION

This invention provides for, in part, novel processes for the C-17 spirolactonization of steroid compounds and novel steroidal compositions produced as intermediates therein.

Accordingly, in a first embodiment, the present invention is directed to a process for the preparation of a 17-spirolactone steroid compound. The process comprises carbonylating a steroid substrate which is substituted at the C-17 position with a first substituent selected from the group consisting of hydroxy and protected hydroxy; and a second substituent selected from the group consisting of alkenyl and alkynyl.

The present invention also encompasses a process for the preparation of a 17-spirolactone steroid compound. The process comprises reducing the 17-alkynyl group of a 17-alkynyl-17-hydroxy steroid compound, or a counterpart compound having a protective group blocking the 17-hydroxyl, to produce a 17-alkenyl-17-hydroxy steroid compound. The process further comprises carbonylating the protected or unprotected 17-alkenyl-17-hydroxy steroid compound to produce the 17-spirolactone steroid compound.

In another embodiment, the present invention is directed to a process for the preparation of a 17-spirolactone steroid compound. The process comprises carbonylating a hydroxyl-protected or unprotected 17-alkynyl-17-hydroxy steroid compound to produce a steroid intermediate comprising a 17-lactenone steroid compound. The process further comprises reducing the 17-lactenone steroid compound of the intermediate to produce a 17-spirolactone steroid compound.

In various embodiments, the present invention is further directed to a process for the preparation of a compound corresponding to the Formula 1503: 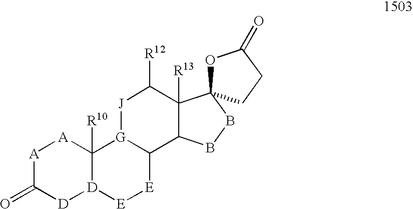
wherein:
R¹⁰, R¹² and R¹³ are independently selected from the group consisting of hydrogen, halo, haloalkyl, hydroxy, alkyl, alkoxy, hydroxyalkyl, alkoxyalkyl, hydroxycarbonyl, cyano and aryloxy;
-A-A- represents the group —CHR¹—CHR²— or —CR¹═CR²—;
where R¹ and R² are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano and aryloxy or R¹ and R² together with the carbons of the steroid backbone to which they are attached form a cycloalkyl group;
—B—B— represents the group —CHR¹⁵—CHR¹⁶—, —CR¹⁵═CR¹⁶— or an α- or β-oriented group: 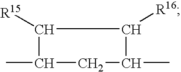
where R¹⁵ and R¹⁶ are independently selected from the group consisting of hydrogen, halo, alkyl, alkoxy, acyl, hydroxyalkyl, alkoxyalkyl, hydroxycarbonyl, alkoxycarbonyl, acyloxyalkyl, cyano, and aryloxy; or
R¹⁵ and R¹⁶, together with the C-15 and C-16 carbons of steroid nucleis to which R¹⁵ and R¹⁶ are respectively attached, form a cycloalkylene group:
-D-D- represents the group: 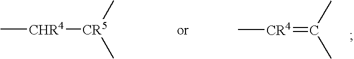
where R⁴ and R⁵ are independently selected from the group consisting of hydrogen, halo, alkyl, alkoxy, acyl, hydroxyalkyl, alkoxyalkyl, hydroxycarbonyl, alkoxycarbonyl, acyloxyalkyl, cyano and aryloxy or R⁴ and R⁵ together with the carbons of the steroid backbone to which they are attached form a cycloalkyl group;
-G-J- represents the group: 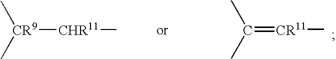
where R⁹ and R¹¹ are independently selected from the group consisting of hydrogen, hydroxy, protected hydroxy, halo, alkyl, alkoxy, acyl, hydroxyalkyl, alkoxyalkyl, hydroxycarbonyl, alkoxycarbonyl, acyloxyalkyl, cyano and aryloxy or R⁹ and R¹¹ together form an epoxy group;
-E-E- represents the group —CHR⁶—CHR⁷— or —CR⁶═CR⁷—;
where R⁶ is selected from the group consisting of hydrogen, halo, alkyl, alkoxy, acyl, hydroxyalkyl, alkoxyalkyl, hydroxycarbonyl, alkoxycarbonyl, acyloxyalkyl, cyano and aryloxy; and
R⁷ is selected from the group consisting of hydrogen, hydroxy, protected hydroxy, halo, alkyl, cycloalkyl, alkoxy, acyl, hydroxyalkyl, alkoxyalkyl, hydroxycarbonyl, alkoxycarbonyl, acyloxyalkyl, cyano, aryloxy, heteroaryl, heterocyclyl, acetylthio, furyl and substituted furyl, or
R⁶ and R⁷, together with the C-6 and C-7 carbons of the steroidal nucleus to which R⁶ and R⁷ are respectively attached, form a cycloalkylene group,
or R⁵ and R⁷, together with the C-5, C-6 and C-7 carbons of the steroid nucleus form a pentacyclic ring fused to the steroid nucleus and comprising a 5,7-lactol, 5,7-hemiacetal or 5,7-lactone corresponding to the structure: 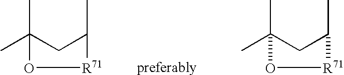
   wherein R⁷¹ comprises ═CH(OH), ═CH(OR⁷²) or ═CH═O.

The process comprises carbonylating a 17-hydroxylprotected or unprotected 17-vinyl-17-hydroxy steroid compound of Formula 1502: 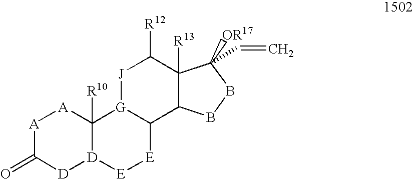
wherein R¹⁷ is hydrogen or a hydroxyl-protecting group, and R¹⁰, R¹², R¹³, -A-A-, —B—B—, -D-D-, -G-J- and -E-E- are as defined above in Formula 1503.

In various other embodiments, the present invention is directed to a process for the preparation of a compound corresponding to the Formula 2503: 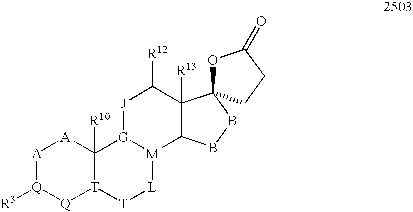
wherein:
R³ is selected from the group consisting of hydrogen, hydroxy, alkoxy, hydroxyalkyl, alkoxyalkyl and hydroxycarbonyl, dihydrocarbylamino, di(substituted hydrocarbyl)amino, and N-heterocyclyl;
-G-J- represents the group 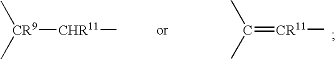
where R⁹ and R¹¹ are independently selected from the group consisting of hydrogen, hydroxy, protected hydroxy, halo, alkyl, alkoxy, acyl, hydroxyalkyl, alkoxyalkyl, hydroxycarbonyl, alkoxycarbonyl, acyloxyalkyl, cyano and aryloxy;
-Q-Q- represents the group 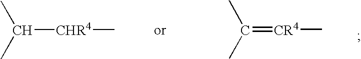
where R⁴ is selected from the group consisting of hydrogen, halo, alkyl, alkoxy, acyl, hydroxyalkyl, alkoxyalkyl, hydroxycarbonyl, alkoxycarbonyl, acyloxyalkyl, cyano and aryloxy; or 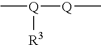
together represent the group 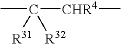
where R³¹ and R³² are independently selected from the group consisting of hydroxy and alkoxy, or R³¹, R³² and the C-3 carbon of the steroid nucleus to which they are attached form the group 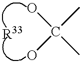
where R³³ is alkylene
-T-T- represents the group 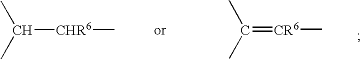
where R⁶ is selected from the group consisting of hydrogen, halo, alkyl, alkoxy, acyl, hydroxyalkyl, alkoxyalkyl, hydroxycarbonyl, alkoxycarbonyl, acyloxyalkyl, cyano and aryloxy;
-L-M- represents the group 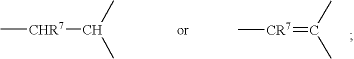
where R⁷ is selected from the group consisting of hydrogen, hydroxy, protected hydroxy, halo, alkyl, cycloalkyl, alkoxy, acyl, hydroxyalkyl, alkoxyalkyl, hydroxycarbonyl, alkoxycarbonyl, acyloxyalkyl, cyano, aryloxy, heteroaryl, heterocyclyl, acetylthio, furyl and substituted furyl;
and R¹⁰, R¹², R¹³, -A-A-, and —B—B— are as defined above for Formula 1503.

The process comprises carbonylating a 17-hydroxyl-protected or -unprotected 17-vinyl-17-hydroxy steroid compound of Formula 2502: 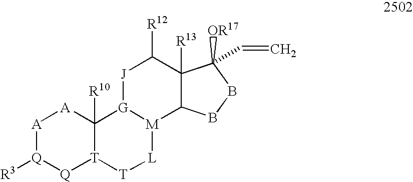
wherein R¹⁷ is as defined above in Formula 1502; R¹⁰, R¹², R¹³, -A-A-, and —B—B— are as defined above for Formula 1503; and R³, -G-J-, -Q-Q-, -T-T-, and -L-M- are as defined above in Formula 2503.

Unless stated otherwise, organic radicals referred to as “lower” in the present disclosure contain at most 7, and preferably from 1 to 4, carbon atoms.

A lower alkoxycarbonyl radical is preferably one derived from an alkyl radical having from 1 to 4 carbon atoms, such as methyl, ethyl, propyl, isopropyl, butyl, isobutyl, sec.-butyl and tert.-butyl; especially preferred are methoxycarbonyl, ethoxycarbonyl and isopropoxycarbonyl. A lower alkoxy radical is preferably one derived from one of the above-mentioned C₁-C₄ alkyl radicals, especially from a primary C₁-C₄ alkyl radical; especially preferred is methoxy. A lower alkanoyl radical is preferably one derived from a straight-chain alkyl having from 1 to 7 carbon atoms; especially preferred are formyl and acetyl.

A methylene bridge in the 15,16-position is preferably β-oriented.

Still further, the present invention is directed to novel steroid compounds of Formulae XXII, XXIV, XXV, XXVI, and XXVII, as described herein below, and the compounds set forth in Table 1.

TABLE 1

| | |
|---|---|
| Formula A XXII | 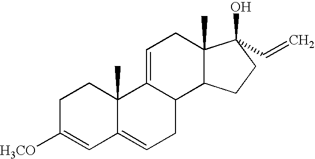 |
| Formula B XXV | 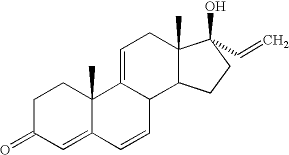 |
| Formula C XXIV | 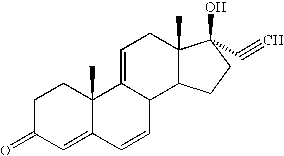 |
| Formula D XXVI | 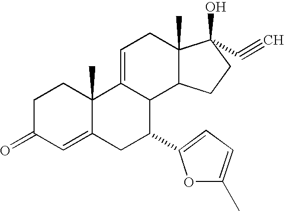 |
| Formula E XXVII | 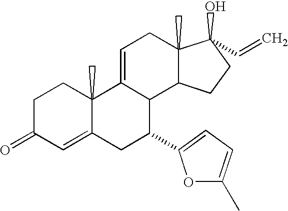 |

Other objects of the invention will be in part apparent and in part pointed out hereinafter.

### DETAILED DESCRIPTION OF THE PREFERRED EMBODIMENTS

In accordance with the present invention, Applicants have discovered a process for the preparation of steroid compounds having a spirolactone moiety at the C-17 position. The process of the present invention generally comprises a carbonylation and a selective hydrogenation of a steroid substrates. An advantage of the process is that the carbonylation and selective hydrogenation reactions may be conducted as isolated steps, in either order, or in situ in a single reaction zone. Further, as is demonstrated below, certain preferred embodiments of the invention provide novel processes for the preparation of epoxymexrenone (methyl hydrogen 9(11)α-epoxy-17α-hydroxy-3-oxopregn-4-ene-7α,21-dicarboxylate, γ-lactone).

It has further been discovered that, in a process scheme for the synthesis of epoxymexrenone, or a 17-spirolactone of similar overall structure, the hydrogenation and carbonylation steps for introduction of the spirolactone group can be integrated with other process steps, such as, for example, 6,7-dehydrogenation of a 3-enol ether-7-furylation, oxidation of a 7α-furyl group to 7α-alkoxycarbonyl, and 9(11)-epoxidation, with a high degree of flexibility as to reaction sequence. By selection of sequence, it is possible to minimize any processing problems that certain steps of such syntheses may otherwise impose on others, and to shift reactions of relatively low yield to early stages of an overall synthesis where the value of the substrate lost is relatively low, thereby minimizing the overall manufacturing cost for the ultimate product.

Steroid Substrate

Steroid substrates for use as starting materials in processes of the present invention generally comprise steroid compounds substituted at the C-17 position with a first substituent selected from the group consisting of hydroxy and protected hydroxy; and a second substituent selected from the group consisting of alkenyl and alkynyl. In preferred embodiments, the steroid substrates are substituted at the C-17 position with a first substituent comprising a hydroxy group and a second substituent comprising an alkenyl or an alkynyl group, more preferably a second substituent comprising a vinyl or an ethynyl group:

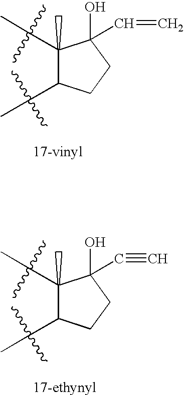

In a first embodiment, the steroid substrate comprises a 17-hydroxy-17-ethynyl steroid or a 17-hydroxyl-protected counterpart thereof comprising a compound of Formula 1501: 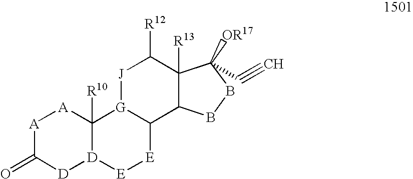
wherein R¹⁷ is as defined above in Formula 1502, and R¹⁰, R¹², R¹³, -A-A-, —B—B—, -G-J-, -D-D-, and -E-E- are as defined above in Formula 1503.

In this and other embodiments, suitable 17-hydroxyl-protective groups include, e.g., alkyl and acyl substituents such as methyl, ethyl, propyl, butyl, phenyl, acetyl, benzyl, xylyl, etc.

In another embodiment, the steroid substrate comprises a 17-hydroxy-17-ethynyl steroid or 17-hydroxyl-protected counterpart thereof comprising a compound of Formula 2501: 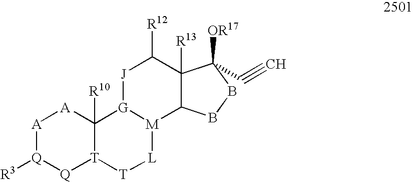
wherein R¹⁷ is as defined above in Formula 1502; R¹⁰, R¹², R¹³, -A-A-, and —B—B— are as defined above for Formula 1503; and R³, -G-J-, -Q-Q-, -T-T-, and -L-M- are as defined above in Formula 2503.

In still another embodiment, the steroid substrate comprises a 17-hydroxy-17-vinyl steroid or a 17-hydroxyl-protected counterpart thereof comprising a compound of Formula 1502: 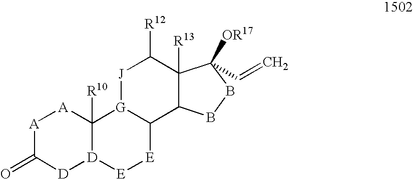
wherein R¹⁰, R¹², R¹³, R¹⁷, -A-A-, —B—B—, -D-D-, -G-J-, and -E-E- are as defined above in Formula 1501.

In another embodiment, the steroid substrate comprises a 17-hydroxy-17-vinyl steroid or a 17-hydroxyl-protected counterpart thereof comprising a compound of Formula 2502: 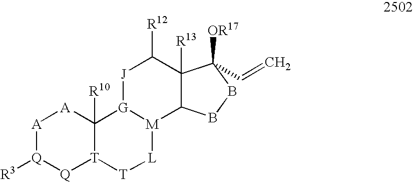
wherein R¹⁷ is as defined above in Formula 1502; R¹⁰, R¹², R¹³, -A-A-, and —B—B— are as defined above for Formula 1503; and R³, -G-J-, -Q-Q-, -T-T-, and -L-M- are as defined above in Formula 2503.

In various preferred embodiments, a 3-keto structure corresponding to formula 1501 or 1502, R¹², R¹⁰ and R¹³ are independently selected from the group consisting of hydrogen, fluoride, chloride, bromide, iodide, fluoromethyl, fluoroethyl, fluoropropyl, fluorobutyl, chloromethyl, chloroethyl, chloropropyl, chlorobutyl, bromomethyl, bromoethyl, bromopropyl, bromobutyl, iodomethyl, iodoethyl, iodopropyl, iodobutyl, hydroxy, methyl, ethyl, straight, branched or cyclic propyl and butyl; methoxy, ethoxy, propoxy, butoxy, hydroxymethyl, hydroxyethyl, hydroxypropyl, hydroxybutyl, methoxymethyl, methoxyethyl, methoxypropyl, methoxybutyl, ethoxymethyl, ethoxyethyl, ethoxypropyl, ethoxybutyl, propoxymethyl, propoxyethyl, propoxypropyl, propoxybutyl, butoxymethyl, butoxyethyl, butoxypropyl, butoxybutyl, hydroxycarbonyl, cyano, phenoxy, benzyloxy;
-A-A- represents the group —CHR¹—CHR²— or —CR¹═CR²;
where R¹ and R² are independently selected from the group consisting of hydrogen, fluoride, chloride, bromide, iodide, methyl, ethyl, propyl, butyl, methoxy, ethoxy, propoxy, butoxy, acetyl, propionyl, butyryl, hydroxymethyl, hydroxyethyl, hydroxypropyl, hydroxybutyl, methoxymethyl, methoxyethyl, methoxypropyl, methoxybutyl, ethoxymethyl, ethoxyethyl, ethoxypropyl, ethoxybutyl, propoxymethyl, propoxyethyl, propoxypropyl, propoxybutyl, butoxymethyl, butoxyethyl, butoxypropyl, butoxybutyl, hydroxycarbonyl, methoxycarbonyl, ethoxycarbonyl, propoxycarbonyl, butoxycarbonyl, acetoxymethyl, acetoxyethyl, acetoxypropyl, acetoxybutyl, propionyloxymethyl, propionyloxyethyl, butyryloxymethyl, butyryloxyethyl, cyano, phenoxy and benzoxy;
or R¹ and R² together with the carbons of the steroid nucleus to which they are attached form a (saturated) cyclopropylene, cyclobutylene, cyclopentylene, cyclohexylene or cycloheptylene group;
—B—B— represents the group —CHR¹⁵—CHR¹⁶—, —CR¹⁵═CR¹⁶— or an α- or β-oriented group: 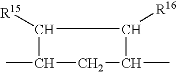
where R¹⁵ and R¹⁶ are independently selected from the group consisting of hydrogen, fluoride, chloride, bromide, iodide, methyl, ethyl, propyl, butyl, methoxy, ethoxy, propoxy, butoxy, acetyl, propionyl, butyryl, hydroxymethyl, hydroxyethyl, hydroxypropyl, hydroxybutyl, methoxymethyl, methoxyethyl, methoxypropyl, methoxybutyl, ethoxymethyl, ethoxyethyl, ethoxypropyl, ethoxybutyl, propoxymethyl, propoxyethyl, propoxypropyl, propoxybutyl, butoxymethyl, butoxyethyl, butoxypropyl, butoxybutyl, hydroxycarbonyl, methoxycarbonyl, ethoxycarbonyl, propoxycarbonyl, butoxycarbonyl, acetoxymethyl, acetoxyethyl, acetoxypropyl, acetoxybutyl, propionyloxymethyl, propionyloxyethyl, butyryloxymethyl, butyryloxyethyl, cyano, phenoxy and benzoxy;
or R¹⁵ and R¹⁶, together with the C-15 and C-16 carbons of the steroid nucelus to which R¹⁵ and R¹⁶ are respectively attached, form a cyclopropylene, cyclobutylene, cyclopentylene, cyclohexylene, cycloheptylene group;
-D-D- represents the group 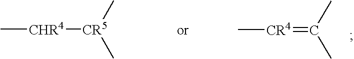
where R⁴ and R⁵ are independently selected from the group consisting of hydrogen, fluoride, chloride, bromide, iodide, methyl, ethyl, propyl, butyl, methoxy, ethoxy, propoxy, butoxy, acetyl, propionyl, butyryl, hydroxymethyl, hydroxyethyl, hydroxypropyl, hydroxybutyl, methoxymethyl, methoxyethyl, methoxypropyl, methoxybutyl, ethoxymethyl, ethoxyethyl, ethoxypropyl, ethoxybutyl, propoxymethyl, propoxyethyl, propoxypropyl, propoxybutyl, butoxymethyl, butoxyethyl, butoxypropyl, butoxybutyl, hydroxycarbonyl, methoxycarbonyl, ethoxycarbonyl, propoxycarbonyl, butoxycarbonyl, acetoxymethyl, acetoxyethyl, acetoxypropyl, acetoxybutyl, propionyloxymethyl, propionyloxyethyl, butyryloxymethyl, butyryloxyethyl, cyano, phenoxy and benzoxy; or R⁴ and R⁵ together with the carbons of the steroid backbone to which they are attached form a cyclopropylene cyclobutylene, cyclopentylene, cyclohexylene, cycloheptylene group;
-G-J- represents the group 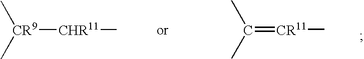
where R⁹ and R¹¹ are independently selected from the group consisting of hydrogen, hydroxy, protected hydroxy, fluoride, chloride, bromide, iodide, methyl, ethyl, propyl, butyl, methoxy, ethoxy, propoxy, butoxy, acetyl, propionyl, butyryl, hydroxymethyl, hydroxyethyl, hydroxypropyl, hydroxybutyl, methoxymethyl, methoxyethyl, methoxypropyl, methoxybutyl, ethoxymethyl, ethoxyethyl, ethoxypropyl, ethoxybutyl, propoxymethyl, propoxyethyl, propoxypropyl, propoxybutyl, butoxymethyl, butoxyethyl, butoxypropyl, butoxybutyl, hydroxycarbonyl, methoxycarbonyl, ethoxycarbonyl, propoxycarbonyl, butoxycarbonyl, acetoxymethyl, acetoxyethyl, acetoxypropyl, acetoxybutyl, propionyloxymethyl, propionyloxyethyl, butyryloxymethyl, butyryloxyethyl, cyano, phenoxy and benzoxy; or R⁹ and R¹¹ together form an epoxy group;
-E-E- represents the group —CHR⁶—CHR⁷— or —CR⁶═CR⁷—, wherein R⁶ is selected from the group consisting of hydrogen, fluoride, chloride, bromide, iodide, methyl, ethyl, propyl, butyl, methoxy, ethoxy, propoxy, butoxy, acetyl, propionyl, butyryl, hydroxymethyl, hydroxyethyl, hydroxypropyl, hydroxybutyl, methoxymethyl, methoxyethyl, methoxypropyl, methoxybutyl, ethoxymethyl, ethoxyethyl, ethoxypropyl, ethoxybutyl, propoxymethyl, propoxyethyl, propoxypropyl, propoxybutyl, butoxymethyl, butoxyethyl, butoxypropyl, butoxybutyl, hydroxycarbonyl, methoxycarbonyl, ethoxycarbonyl, propoxycarbonyl, butoxycarbonyl, acetoxymethyl, acetoxyethyl, acetoxypropyl, acetoxybutyl, propionyloxymethyl, propionyloxyethyl, butyryloxymethyl, butyryloxyethyl, cyano, phenoxy and benzoxy; and
R⁷ is selected from the group consisting of hydrogen, hydroxyl, protected hydroxyl, fluoride, chloride, bromide, iodide, methyl, ethyl, propyl, butyl, cyclopropyl, cyclobutyl, cyclopentyl, cyclohexyl, cycloheptyl, methoxy, ethoxy, propoxy, butoxy, acetyl, propionyl, butyryl, hydroxymethyl, hydroxyethyl, hydroxypropyl, hydroxybutyl, methoxymethyl, methoxyethyl, methoxypropyl, methoxybutyl, ethoxymethyl, ethoxyethyl, ethoxypropyl, ethoxybutyl, propoxymethyl, propoxyethyl, propoxypropyl, propoxybutyl, butoxymethyl, butoxyethyl, butoxypropyl, butoxybutyl, hydroxycarbonyl, methoxycarbonyl, ethoxycarbonyl, propoxycarbonyl, butoxycarbonyl, acetoxymethyl, acetoxyethyl, acetoxypropyl, acetoxybutyl, propionyloxymethyl, propionyloxyethyl, butyryloxymethyl, butyryloxyethyl, cyano, phenoxy, benzoxy, pyrrolyl, imidazolyl, thiazolyl, pyridyl, pyrimidyl, oxazolyl, acetylthio, furyl, substituted furyl, thienyl and substituted thienyl;
or R⁶ and R⁷, together with the C-6 and C-7 carbons of the steroid nucleus to which R⁶ and R⁷ are respectively attached, form a (saturated) cyclopropylene cyclobutylene, cyclopentylene, cyclohexylene, cycloheptylene group.

In many of such embodiments,
R¹² is selected from the group consisting of hydrogen, fluoride, chloride, bromide, iodide, fluoromethyl, fluoroethyl, fluoropropyl, fluorobutyl, chloromethyl, chloroethyl, chloropropyl, chlorobutyl, bromomethyl, bromoethyl, bromopropyl, bromobutyl, iodomethyl, iodoethyl, iodopropyl, iodobutyl, hydroxy, methyl, ethyl, straight, branched or cyclic propyl and butyl; methoxy, ethoxy, propoxy, butoxy, hydroxymethyl, hydroxyethyl, hydroxypropyl, hydroxybutyl, and cyano;
R¹⁰ and R¹³ are methyl;
-A-A- represents the group —CH₂—CH₂— or —CH═CH—;
—B—B— represents the group —CHR¹⁵—CHR¹⁶—, —CR¹⁵═CR¹⁶— or an α- or β-oriented group: 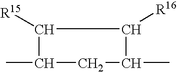
where R¹⁵ and R¹⁶ are independently selected from the group consisting of hydrogen, fluoride, chloride, bromide, iodide, methyl, ethyl, propyl, butyl, methoxy, ethoxy, propoxy, butoxy, acetyl, propionyl, butyryl, hydroxymethyl, hydroxyethyl, hydroxypropyl, hydroxybutyl and cyano;
or R¹⁵ and R¹⁶, together with the C-15 and C-16 carbons of the steroid nucelus to which R¹⁵ and R¹⁶ are respectively attached, form a cyclopropylene, cyclobutylene, cyclopentylene, cyclohexylene, cycloheptylene group;
-D-D- represents the group 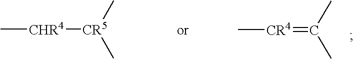
where R⁴ and R⁵ are independently selected from the group consisting of hydrogen, fluoride, chloride, bromide, iodide, methyl, ethyl, propyl, butyl, methoxy, ethoxy, propoxy, butoxy, acetyl, propionyl, butyryl, hydroxymethyl, hydroxyethyl, hydroxypropyl, hydroxybutyl and cyano;
G-J- represents the group 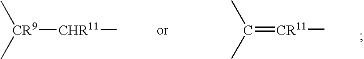
where R⁹ and R¹¹ are hydrogen; or R⁹ and R¹¹ together form an epoxy group;
-E-E- represents the group —CHR⁶—CHR⁷— or —CR⁶═CR⁷—, wherein R⁶ is selected from the group consisting of hydrogen, fluoride, chloride, bromide, iodide, methyl, ethyl, propyl, butyl, methoxy, ethoxy, propoxy, butoxy, acetyl, propionyl, butyryl, hydroxymethyl, hydroxyethyl, hydroxypropyl, hydroxybutyl and cyano; and
R⁷ is selected from the group consisting of hydrogen, hydroxyl, protected hydroxyl, fluoride, chloride, bromide, iodide, methyl, ethyl, propyl, butyl, cyclopropyl, cyclobutyl, cyclopentyl, cyclohexyl, cycloheptyl, methoxy, ethoxy, propoxy, butoxy, acetyl, propionyl, butyryl, hydroxymethyl, hydroxyethyl, hydroxypropyl, hydroxybutyl, cyano, furyl, thienyl, substituted furyl and substituted thienyl;
or R⁶ and R⁷, together with the C-6 and C-7 carbons of the steroid nucleus to which R⁶ and R⁷ are respectively attached, form a (saturated) cyclopropylene cyclobutylene, cyclopentylene, cyclohexylene, cycloheptylene group,
or R⁵ and R⁷, together with the C-5, C-6 and C-7 carbons of the steroid nucleus form a pentacyclic ring fused to the steroid nucleus and comprising a 5,7-lactol, 5,7-hemiacetal or 5,7-lactone corresponding to the structure: 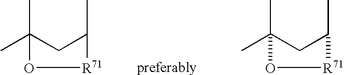
   wherein R⁷¹ comprises ═CH(OH), ═CH(OR⁷²) or ═CH═O.

In various preferred embodiments, R¹² is selected from the group consisting of hydrogen, halo, hydroxy, alkyl, alkoxy, hydroxyalkyl, alkoxyalkyl, hydroxycarbonyl, cyano and aryloxy;
R¹⁰ and R¹³ are methyl, particularly β-methyl;
-A-A- represents the group —CH₂—CH₂—;
—B—B— represents the group —CHR¹⁵—CHR¹⁶—; where R¹⁵ and R¹⁶ are hydrogen;
or R¹⁵ and R¹⁶, together with the C-15 and C-16 carbons of the steroid nucleus to which they are respectively attached, form a (saturated) cycloalkylene group;
-D-D- represents the group: 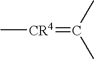
where R⁴ is hydrogen;
-E-E- represents the group —CHR⁶—CHR⁷—; where R⁶ is hydrogen;
where R⁷ is selected from the group consisting of hydrogen, furyl, substituted furyl, thienyl, substituted thienyl and acetylthio;
or R⁶ and R⁷, together with the C-6 and C-7 carbons of the steroid nucleus to which they are respectively attached, form a (saturated) cycloalkylene group;
-J-G- represents the group 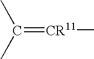
where R¹¹ is hydrogen.

In compounds of formulae 1501 and 1502, particular exemplary substituents which may constitute R³ include hydrogen, hydroxy, methoxy, ethoxy, propoxy, butoxy, hydroxymethyl, hydroxyethyl, hydroxypropyl, hydroxybutyl, methoxymethyl, methoxyethyl, methoxypropyl, methoxybutyl, ethoxymethyl, ethoxyethyl, ethoxypropyl, ethoxybutyl, propoxymethyl, propoxyethyl, propoxypropyl, propoxybutyl, butoxymethyl, butoxyethyl, butoxypropyl, butoxybutyl, hydroxycarbonyl, N,N dimethylamino, N,N-diethylamino, N,N-dipropylamino, N,N-dibutylamino, N,N-diallylamino, N,N-diphenylamino, N-pyrrolidinyl, N-piperidinyl and N-morpholino.

Preferably, R³ is selected from the group consisting of methoxy, ethoxy, propoxy and butoxy.

R¹⁰, R¹² and R¹³ are independently selected from the group consisting of hydrogen, fluoride, chloride, bromide, iodide, fluoromethyl, fluoroethyl, fluoropropyl, fluorobutyl, chloromethyl, chloroethyl, chloropropyl, chlorobutyl, bromomethyl, bromoethyl, bromopropyl, bromobutyl, iodomethyl, iodoethyl, iodopropyl, iodobutyl, hydroxy, methyl, ethyl, straight, branched or cyclic propyl and butyl; methoxy, ethoxy, propoxy, butoxy, hydroxymethyl, hydroxyethyl, hydroxypropyl, hydroxybutyl, methoxymethyl, methoxyethyl, methoxypropyl, methoxybutyl, ethoxymethyl, ethoxyethyl, ethoxypropyl, ethoxybutyl, propoxymethyl, propoxyethyl, propoxypropyl, propoxybutyl, butoxymethyl, butoxyethyl, butoxypropyl, butoxybutyl, hydroxycarbonyl, cyano, phenoxy, benzyloxy;
-A-A- represents the group —CHR¹—CHR²— or —CR¹═CR²—;
where R¹ and R² are independently selected from the group consisting of hydrogen, fluoride, chloride, bromide, iodide, methyl, ethyl, propyl, butyl, methoxy, ethoxy, propoxy, butoxy, acetyl, propionyl, butyryl, hydroxymethyl, hydroxyethyl, hydroxypropyl, hydroxybutyl, methoxymethyl, methoxyethyl, methoxypropyl, methoxybutyl, ethoxymethyl, ethoxyethyl, ethoxypropyl, ethoxybutyl, propoxymethyl, propoxyethyl, propoxypropyl, propoxybutyl, butoxymethyl, butoxyethyl, butoxypropyl, butoxybutyl, hydroxycarbonyl, methoxycarbonyl, ethoxycarbonyl, propoxycarbonyl, butoxycarbonyl, acetoxymethyl, acetoxyethyl, acetoxypropyl, acetoxybutyl, propionyloxymethyl, propionyloxyethyl, butyryloxymethyl, butyryloxyethyl, cyano, phenoxy and benzoxy;
or R¹ and R² together with the carbons of the steroid nucleus to which they are attached form a (saturated) cyclopropylene, cyclobutylene, cyclopentylene, cyclohexylene or cycloheptylene group;
—B—B— represents the group —CHR¹⁵—CHR¹⁶—, CR¹⁵═CR¹⁶— or an α- or β-oriented group: 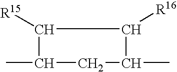
where R¹⁵ and R¹⁶ are independently selected from the group consisting of hydrogen, fluoride, chloride, bromide, iodide, methyl, ethyl, propyl, butyl, methoxy, ethoxy, propoxy, butoxy, acetyl, propionyl, butyryl, hydroxymethyl, hydroxyethyl, hydroxypropyl, hydroxybutyl, methoxymethyl, methoxyethyl, methoxypropyl, methoxybutyl, ethoxymethyl, ethoxyethyl, ethoxypropyl, ethoxybutyl, propoxymethyl, propoxyethyl, propoxypropyl, propoxybutyl, butoxymethyl, butoxyethyl, butoxypropyl, butoxybutyl, hydroxycarbonyl, methoxycarbonyl, ethoxycarbonyl, propoxycarbonyl, butoxycarbonyl, acetoxymethyl, acetoxyethyl, acetoxypropyl, acetoxybutyl, propionyloxymethyl, propionyloxyethyl, butyryloxymethyl, butyryloxyethyl, cyano, phenoxy and benzoxy;
or R¹⁵ and R¹⁶, together with the C-15 and C-16 carbons of the steroid nucelus to which R¹⁵ and R¹⁶ are respectively attached, form a cyclopropylene, cyclobutylene, cyclopentylene, cyclohexylene, cycloheptylene group;
-G-J- represents the group 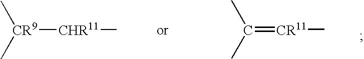
where R⁹ and R¹¹ are independently selected from the group consisting of hydrogen, hydroxy, protected hydroxy, fluoride, chloride, bromide, iodide, methyl, ethyl, propyl, butyl, methoxy, ethoxy, propoxy, butoxy, acetyl, propionyl, butyryl, hydroxymethyl, hydroxyethyl, hydroxypropyl, hydroxybutyl, methoxymethyl, methoxyethyl, methoxypropyl, methoxybutyl, ethoxymethyl, ethoxyethyl, ethoxypropyl, ethoxybutyl, propoxymethyl, propoxyethyl, propoxypropyl, propoxybutyl, butoxymethyl, butoxyethyl, butoxypropyl, butoxybutyl, hydroxycarbonyl, methoxycarbonyl, ethoxycarbonyl, propoxycarbonyl, butoxycarbonyl, acetoxymethyl, acetoxyethyl, acetoxypropyl, acetoxybutyl, propionyloxymethyl, propionyloxyethyl, butyryloxymethyl, butyryloxyethyl, cyano, phenoxy and benzoxy;
-Q-Q- represents the group 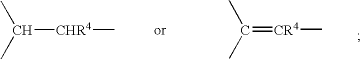
where R⁴ is selected from the group consisting of hydrogen, fluoride, chloride, bromide, iodide, methyl, ethyl, propyl, butyl, methoxy, ethoxy, propoxy, butoxy, acetyl, propionyl, butyryl, hydroxymethyl, hydroxyethyl, hydroxypropyl, hydroxybutyl, methoxymethyl, methoxyethyl, methoxypropyl, methoxybutyl, ethoxymethyl, ethoxyethyl, ethoxypropyl, ethoxybutyl, propoxymethyl, propoxyethyl, propoxypropyl, propoxybutyl, butoxymethyl, butoxyethyl, butoxypropyl, butoxybutyl, hydroxycarbonyl, methoxycarbonyl, ethoxycarbonyl, propoxycarbonyl, butoxycarbonyl, acetoxymethyl, acetoxyethyl, acetoxypropyl, acetoxybutyl, propionyloxymethyl, propionyloxyethyl, butyryloxymethyl, butyryloxyethyl, cyano, phenoxy and benzoxy; or 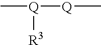
together represent the group 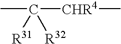
where R³¹ and R³² are independently selected from the group consisting of hydroxy, methoxy, ethoxy, propoxy and butoxy; or R³¹, R³² and the C-3 carbon of the steroid nucleus to which they are attached form the group 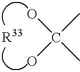
where R³³ is substituted or unsubstituted ethylene, propylene and butylenes.
-T-T- represents the group 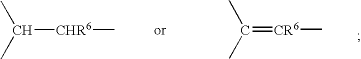
where R⁶ is selected from the group consisting of hydrogen, fluoride, chloride, bromide, iodide, methyl, ethyl, propyl, butyl, methoxy, ethoxy, propoxy, butoxy, acetyl, propionyl, butyryl, hydroxymethyl, hydroxyethyl, hydroxypropyl, hydroxybutyl, methoxymethyl, methoxyethyl, methoxypropyl, methoxybutyl, ethoxymethyl, ethoxyethyl, ethoxypropyl, ethoxybutyl, propoxymethyl, propoxyethyl, propoxypropyl, propoxybutyl, butoxymethyl, butoxyethyl, butoxypropyl, butoxybutyl, hydroxycarbonyl, methoxycarbonyl, ethoxycarbonyl, propoxycarbonyl, butoxycarbonyl, acetoxymethyl, acetoxyethyl, acetoxypropyl, acetoxybutyl, propionyloxymethyl, propionyloxyethyl, butyryloxymethyl, butyryloxyethyl, cyano, phenoxy and benzoxy; and
-L-M- represents the group 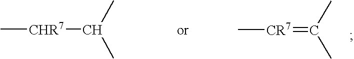
where R⁷ is selected from the group consisting of hydrogen, hydroxyl, protected hydroxyl, fluoride, chloride, bromide, iodide, methyl, ethyl, propyl, butyl, cyclopropyl, cyclobutyl, cyclopentyl, cyclohexyl, cycloheptyl, methoxy, ethoxy, propoxy, butoxy, acetyl, propionyl, butyryl, hydroxymethyl, hydroxyethyl, hydroxypropyl, hydroxybutyl, methoxymethyl, methoxyethyl, methoxypropyl, methoxybutyl, ethoxymethyl, ethoxyethyl, ethoxypropyl, ethoxybutyl, propoxymethyl, propoxyethyl, propoxypropyl, propoxybutyl, butoxymethyl, butoxyethyl, butoxypropyl, butoxybutyl, hydroxycarbonyl, methoxycarbonyl, ethoxycarbonyl, propoxycarbonyl, butoxycarbonyl, acetoxymethyl, acetoxyethyl, acetoxypropyl, acetoxybutyl, propionyloxymethyl, propionyloxyethyl, butyryloxymethyl, butyryloxyethyl, cyano, phenoxy, benzoxy, pyrrolyl, imidazolyl, thiazolyl, pyridyl, pyrimidyl, oxazolyl, acetylthio, furyl, substituted furyl, thienyl and substituted thienyl;
or R⁶ and R⁷, together with the C-6 and C-7 carbons of the steroid nucleus to which they are respectively attached, form a (saturated) cyclopropylene, cyclobutylene, cyclopenylene or cyclohexylene group,
or R⁵ and R⁷, together with the C-5, C-6 and C-7 carbons of the steroid nucleus form a pentacyclic ring fused to the steroid nucleus and comprising a 5,7-lactol, 5,7-hemiacetal or 5,7-lactone corresponding to the structure: 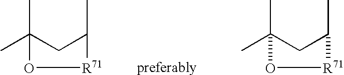
   wherein R⁷¹ comprises ═CH(OH), ═CH(OR⁷²) or ═CH═O.

In many of such embodiments,
R³ is selected from the group consisting of hydrogen, hydroxy, methoxy, ethoxy, propoxy, butoxy, hydroxymethyl, hydroxyethyl, hydroxypropyl, hydroxybutyl, hydroxycarbonyl, N,N dimethylamino, N,N-diethylamino, N,N-dipropylamino, N,N-dibutylamino, N,N-diallylamino, N,N-diphenylamino, N-pyrrolidinyl, N-piperidinyl and N-morpholino;
R¹² is selected from the group consisting of hydrogen, fluoride, chloride, bromide, iodide, fluoromethyl, fluoroethyl, fluoropropyl, fluorobutyl, chloromethyl, chloroethyl, chloropropyl, chlorobutyl, bromomethyl, bromoethyl, bromopropyl, bromobutyl, iodomethyl, iodoethyl, iodopropyl, iodobutyl, hydroxy, methyl, ethyl, straight, branched or cyclic propyl and butyl; methoxy, ethoxy, propoxy, butoxy, hydroxymethyl, hydroxyethyl, hydroxypropyl, hydroxybutyl and cyano;
R¹⁰ and R¹³ are methyl;
-A-A- represents the group —CHR¹—CHR²— or —CR¹═CR²—;
where R¹ and R² are independently selected from the group consisting of hydrogen, fluoride, chloride, bromide, iodide, methyl, ethyl, propyl, butyl, methoxy, ethoxy, propoxy, butoxy, acetyl, propionyl, butyryl, hydroxymethyl, hydroxyethyl, hydroxypropyl, hydroxybutyl and cyano;
or R¹ and R² together with the carbons of the steroid nucleus to which they are attached form a (saturated) cyclopropylene, cyclobutylene, cyclopentylene, cyclohexylene or cycloheptylene group;
—B—B— represents the group —CHR¹⁵—CHR¹⁶—, —CR¹⁵═CR¹⁶— or an α- or β-oriented group: 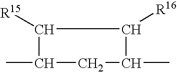
where R¹⁵ and R¹⁶ are independently selected from the group consisting of hydrogen, fluoride, chloride, bromide, iodide, methyl, ethyl, propyl, butyl, methoxy, ethoxy, propoxy, butoxy, acetyl, propionyl, butyryl, hydroxymethyl, hydroxyethyl, hydroxypropyl, hydroxybutyl and cyano;
or R¹⁵ and R¹⁶, together with the C-15 and C-16 carbons of the steroid nucelus to which R¹⁵ and R¹⁶ are respectively attached, form a cyclopropylene, cyclobutylene, cyclopentylene, cyclohexylene, cycloheptylene group;
-G-J- represents the group 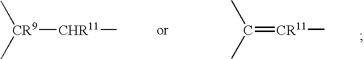
where R⁹ and R¹¹ are independently selected from the group consisting of hydrogen, hydroxy, protected hydroxy, fluoride, chloride, bromide, iodide, methyl, ethyl, propyl, butyl, methoxy, ethoxy, propoxy, butoxy, acetyl, propionyl, butyryl, hydroxymethyl, hydroxyethyl, hydroxypropyl, hydroxybutyl and cyano;
-Q-Q- represents the group 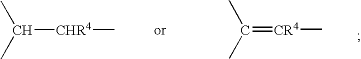
where R⁴ is selected from the group consisting of hydrogen, fluoride, chloride, bromide, iodide, methyl, ethyl, propyl, butyl, methoxy, ethoxy, propoxy, butoxy, acetyl, propionyl, butyryl, hydroxymethyl, hydroxyethyl, hydroxypropyl, hydroxybutyl and cyano; or 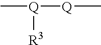
together represent the group 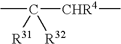
where R³¹ and R³² are independently selected from the group
consisting of hydroxy, methoxy, ethoxy, propoxy and butoxy; or R³¹, R³² and the C-3 carbon of the steroid nucleus to which they are attached form the group 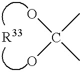
where R³³ is substituted or unsubstituted ethylene, propylene and butylenes.
-T-T- represents the group 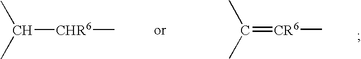
where R⁶ is selected from the group consisting of hydrogen, fluoride, chloride, bromide, iodide, methyl, ethyl, propyl, butyl, methoxy, ethoxy, propoxy, butoxy, acetyl, propionyl, butyryl, hydroxymethyl, hydroxyethyl, hydroxypropyl, hydroxybutyl and cyano; and
-L-M- represents the group 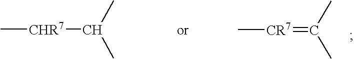
where R⁷ is selected from the group consisting of hydrogen, hydroxyl, protected hydroxyl, fluoride, chloride, bromide, iodide, methyl, ethyl, propyl, butyl, cyclopropyl, cyclobutyl, cyclopentyl, cyclohexyl, cycloheptyl, methoxy, ethoxy, propoxy, butoxy, acetyl, propionyl, butyryl, hydroxymethyl, hydroxyethyl, hydroxypropyl, hydroxybutyl, cyano, pyrrolyl, imidazolyl, thiazolyl, pyridyl, pyrimidyl, oxazolyl, acetylthio, furyl, substituted furyl, thienyl and substituted thienyl;
or R⁶ and R⁷, together with the C-6 and C-7 carbons of the steroid nucleus to which they are respectively attached, form a (saturated) cyclopropylene, cyclobutylene, cyclopenylene or cyclohexylene group.
or R⁵ and R⁷, together with the C-5, C-6 and C-7 carbons of the steroid nucleus form a pentacyclic ring fused to the steroid nucleus and comprising a 5,7-lactol, 5,7-hemiacetal or 5,7-lactone corresponding to the structure: 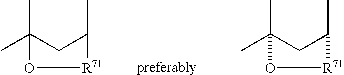
   wherein R⁷¹ comprises ═CH(OH), ═CH(OR⁷²) or ═CH═O.

In various preferred embodiments, R³ is selected from the group consisting of hydrogen, hydroxy, alkoxy, hydroxyalkyl, alkoxyalkyl and hydroxycarbonyl, dihydrocarbylamino, di(substituted hydrocarbyl)amino and N-heterocyclyl;
R¹² is selected from the group consisting of hydrogen, halo, hydroxy, alkyl, alkoxy, hydroxyalkyl, alkoxyalkyl, hydroxycarbonyl, cyano and aryloxy;
R¹⁰ and R¹³ are methyl;
-A-A- represents the group —CH₂—CH₂—;
-G-J- represents the group 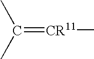
where R¹¹ is hydrogen;
-Q-Q- represents the group 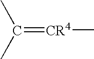
where R⁴ is hydrogen;
-T-T- represents the group 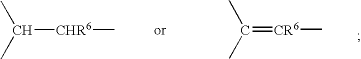
where R⁶ is hydrogen;
-L-M- represents the group 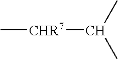
where R⁷ is selected from the group consisting of hydrogen, acetylthio, furyl, substituted furyl, thienyl and substituted thienyl;
or R⁶ and R⁷, together with the C-6 and C-7 carbons of the steroid nucleus to which they are respectively attached, form a (saturated) cycloalkylene group;
—B—B— represents the group —CHR¹⁵—CHR¹⁶—; where R¹⁵ and R¹⁶ are hydrogen;
or R¹⁵ and R¹⁶, together with the C-15 and C-16 carbons of the steroid nucleus to which they are respectively attached, form a (saturated) cycloalkylene group.

As described above, the process of the present invention generally comprises the steps of carbonylation and selective hydrogenation to incorporate a spirolactone moiety at the C-17 position of a steroid compound. An advantage of the process is that the carbonylation and selective hydrogenation reactions may be conducted as isolated steps, in either order, or in situ in a single reaction zone, thereby providing a flexible method which may be utilized on a wide variety of substrates as described above. For example, in a certain preferred embodiment beginning with a 17-ethynyl steroid substrate, the process may comprise two alternative reaction sequences including a carbonylation followed by a hydrogenation as shown in Reaction Scheme A or a hydrogenation followed by a carbonylation as shown in Reaction Scheme B.

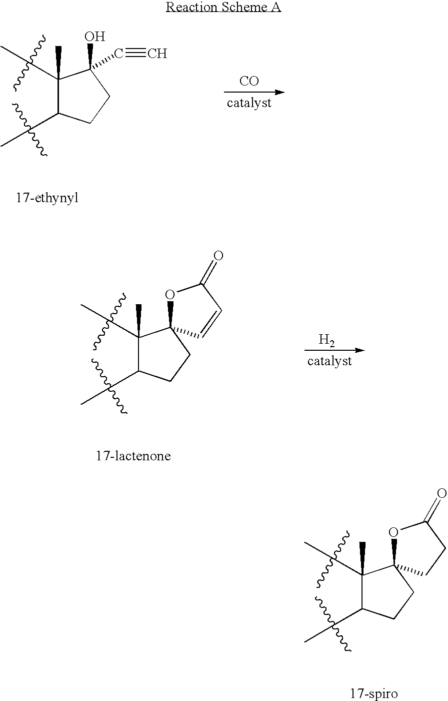

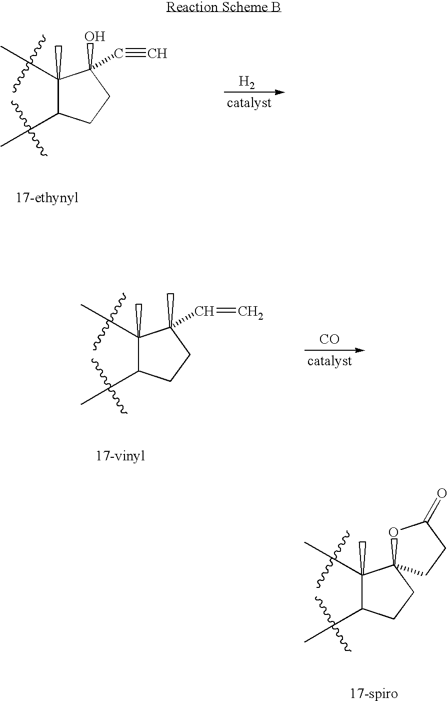

In further alternatives, other process steps can precede, follow, or intervene between the hydrogenation and carbonylation reactions. Prominently included in such reactions schemes are steps integral to various alternative processes for the manufacture of eplerenone.

A. Carbonylation

In various of its embodiments, the process of the present invention comprises carbonylating steroid substrates substituted at the C-17 position. For example, a 17-hydroxy-17-vinyl substrate or its 17-hydroxyl-protected counterpart may be catalytically reacted with CO to form a 17-spirobutyrolactone. As described herein, a 17-hydroxy-17-vinyl intermediate may be prepared by catalytic hydrogenation of a 17-hydroxy-17-ethynyl steroid substrate. Alternatively, a 17-hydroxy-17-ethynyl substrate may be directly carbonylated to yield a reaction mass comprising a 17-spirolactone.

Where the substrate for the carbonylation is a 17-ethynyl steroid, the reaction mass typically comprises a mixture of the 17-spirolactone and the 17-lactenone. As discussed herein, the carbonylation reaction is conducted in the presence of a reducing agent which is effective for the formation of the reaction catalyst. Without being held to a particular theory, it is believed that the reducing agent is also effective under the reaction conditions for partial reduction of the 17-ethynyl to the 17-vinyl, with the latter intermediate being converted to the spirolactone and the former to the lactenone. As further described hereinbelow, the lactenone may be converted to the spirolactone by further reduction, e.g., by catalytic hydrogenation. In a reaction sequence that may be more preferred for many embodiments of the invention, a 17-ethynyl substrate is reduced to a 17-vinyl steroid prior to carbonylation, e.g., by catalytic hydrogenation, as also described in more detail below.

Generally, the carbonylation reaction comprises contacting the steroid substrate with a source of carbon monoxide and a carbonylation catalyst. Typically, the carbonylation catalyst comprises a metal catalyst, preferably a metal selected from the group consisting of Co, Ni, Fe, Pt, Pd, Ru, Rh, Ir and mixtures thereof, with Pd being preferred in certain embodiments.

In accordance with the present invention, it has further been discovered that an active carbonylation catalyst species can be generated in situ in a carbonylation reaction medium, typically a medium comprising a solvent for the steroid substrate. For example, the carbonylation catalyst may be formed by contacting a source of a metal with a source of carbon monoxide, preferably together with another reducing agent. In other preferred embodiments the catalyst may be formed by contacting a source of metal with carbon monoxide in the presence of a ligand and/or a reducing agent.

When the catalyst comprises palladium, suitable palladium sources may comprise palladium acetate, PdCl₂, PdO, Pd/C, or a coordination catalyst such as PdCl₂(PPh₃)₂, Pd(dba)₂, or Pd₂(dba)₃. For example, palladium on carbon has been used successfully in carbonylation reactions as a source of the homogeneous catalytic species. However, use of Pd/C generates a spent carbon support that must be later removed from the product mixture by filtration. Thus, in certain embodiments, palladium acetate is preferred because of its stability, availability, cost, reliability, and versatility.

Further, in embodiments wherein the reaction is conducted in the presence of a source of Pd such as palladium acetate, PdO, PdCl₂, or Pd/C, it may be preferred to contact the metal with a ligand such as a ligand containing phosphorus. Examples of suitable phosphorus containing ligands include phosphine ligands, preferably phosphine ligands selected from the group consisting of dppb, bdpp, dppf, DPEphos, and xantphos.

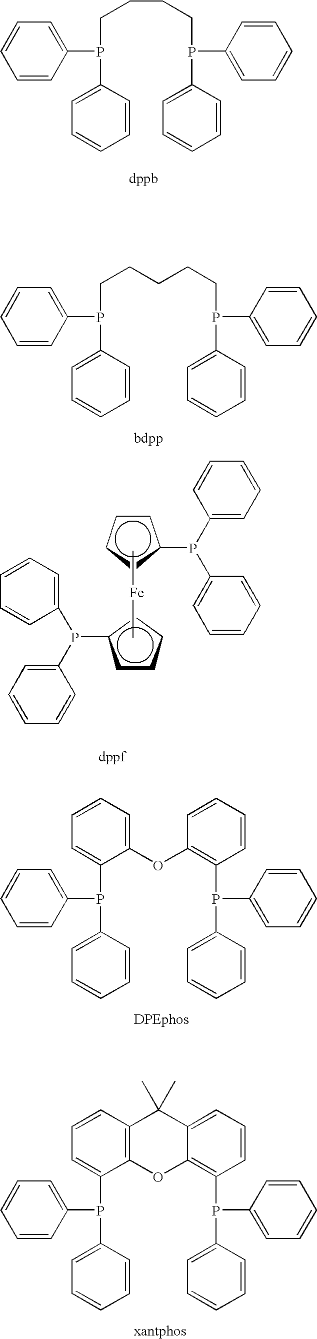

Suitable reducing agents for use in forming the catalyst may generally comprise any active hydrogen source known to those skilled in the art, with active hydrogen sources such as hydrogen, formic acid, borohydrides and oxalic acid being preferred in some embodiments.

The carbonylation reaction may be conducted in a reaction system comprising a liquid medium comprising a solvent for the steroid substrate. Preferred solvents are selected from among those in which the substrate steroid, typically either a 17β-hydroxy-17α-ethynyl steroid, a 17β-hydroxy-17α-vinyl steroid, or a 17-hydroxyl-protected counterpart of either of these, has a reasonable solubility. For example, suitable solvents typically comprise a solvent selected from the group consisting of methylene chloride, tetrahydrofuran, ethyl acetate, acetonitrile, dimethylether, dioxane, toluene, dimethylformamide and mixtures thereof. The concentration of steroid substrate in the liquid reaction medium is typically between about 0.1% and about 60% by weight, preferably at least about 5% by weight, conveniently between about 10% and about 30% by weight. The catalyst may be dissolved or dispersed in the solvent for the steroid substrate, typically at a concentration in the range of about 0.0001 and about 10 mole %, preferably between about 0.01 and about 10 mole %, as measured by the charge of noble metal relative to the charge of steroid substrate.

Preferably, the carbonylation reaction is carried out under a CO partial pressure of at least about 5 psia, typically between about 0 psig and about 500 psig, and at a temperature in the range of about 20 to about 170° C., more typically between about 95° and about 130° C. In order to avoid a higher than necessary total pressure at the requisite CO partial pressure, the solvent for the steroid substrate is preferably selected from among solvents that do not exhibit an excessive vapor pressure at the temperature of the reaction. Based on a combination of favorable properties, THF and dioxane are preferred solvents.

Without being held to a particular theory, it is believed that the carbonylation reaction proceeds according to Scheme C set forth below, wherein elemental Pd is reduced to a hydride which forms a catalytic co-ordination complex (A) with the added ligands and solvent. Contact of the coordination catalyst (A) with the steroid substrate results in the formation of complex (B) wherein the solvent has been displaced by the substrate, and Pd is coordinated to the 17-vinyl or 17-ethynyl moiety of the steroid. The reaction is then believed to proceed via carbonylation of the steroid/Pd hydride complex (B) to displace the hydride with a carbonyl group while reducing the unsaturated bond. Rearrangement of the resulting complex (C) forms an α-carbonylethyl or α-carbonylvinyl substituent at C-17 of the steroid, the rearranged structure (D) remaining coordinated to the Pd via a carbonyl moiety. Ring closures yields the 17-spirolactone (E) where the 17-substituent of the initial substrate is vinyl, or the lactenone (not shown) where the 17-substituent of the substrate is ethynyl. Ring closure releases the Pd with reformation of the Pd hydride coordination catalyst for further reaction with the substrate.

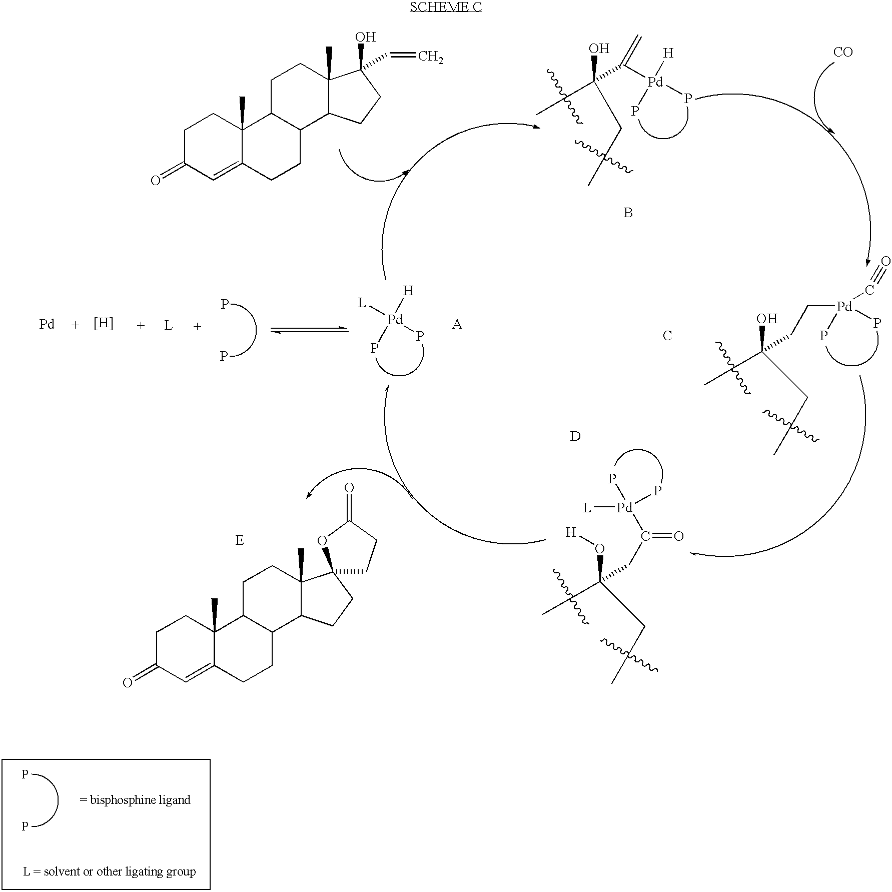

Regardless of the precise mechanism of the carbonylation, the net effect is reaction of carbon monoxide with the 17-vinyl group to yield a 17-carboxylic group, which may combine with the 17-hydroxy to form the lactone, or remain open in the form of a carboxylate salt. As illustrated in Scheme 3, it is believed that the catalyst and CO form a complex with the 17-hydrocarbyl, from which the catalyst complex then dissociates leaving a carboxyl anion which may combine with the 17-hydroxyl to form the lactone. Under acidic to neutral conditions, the ring closes and the lactone is formed. The carbonylation reaction, thus, differs from the hydroformylation reaction of Wuts, et al., J. Org. Chem. 1989, 54, 5180-5182 wherein a 17β-hydroxy-17α-ethynyl steroid reacts with both carbon monoxide and hydrogen to form the 17-lactol rather than the 17-lactone. Wuts then converts the lactol to the lactone by use of an oxidizing agent.

In the carbonylation as described and claimed herein, hydrogen may serve one or more roles, but does not function primarily to reduce the substrate or intermediate species in such a way as to yield predominantly the lactol rather than the lactone. In the carbonylation reaction per se, as described herein, the principal role of hydrogen is understood to be the stabilization of the carbonylation catalyst. In the case of in situ reduction of the 17-ethynyl and carbonylation of the 17-vinyl group, hydrogen also functions to reduce the ethynyl to vinyl prior to carbonylation and/or to reduce a lactenone by-product to lactone. It does not function primarily to produce the lactol rather than the lactone. The process as herein described may not in all cases quantitatively avoid formation of any lactol, but it predominantly yields the lactone or carboxylate salt.

B. Hydrogenation

In various preferred embodiments, the process of the present invention comprises the selective hydrogenation of 17-alkynyl steroids as defined above. In these embodiments, the process comprises contacting the steroid substrate with a source of hydrogen, more preferably in the presence of a catalyst. The hydrogenation reaction produces a 17-vinyl steroid, e.g., a 17-hydroxy-17-vinyl steroid which can serve as a substrate for the carbonylation reaction described above for the preparation of a 17-spirolactone.

Preferred catalysts for the hydrogenation reaction typically comprise noble metals, such as noble metals on carbon or calcium carbonate supports. Other supports such as silica, alumina and zeolites can also be used. An example of a certain preferred noble metal catalyst comprises palladium on a calcium carbonate support such as a “Lindlar” catalyst. Lindlar catalysts are known in the art and available commercially, for example, from Johnson Matthey and Sigma Aldrich. A preferred type of Lindlar catalyst is Johnson Matthey type A310050-5 comprising 5% by weight Pd on a calcium carbonate support poisoned by lead. The loading of Pb, for example as lead acetate, is adjusted to attenuate the activity of the catalyst so that it remains active for the reduction of ethynyl to vinyl but relatively inactive for the further reduction of the 17-vinyl group to ethyl, or for any other side reactions that could otherwise possibly occur. An appropriate concentration of Pb source for adjustment of catalyst activity can be readily identified by one skilled in the art for any particular combination of substrate species, catalyst species, concentration, temperature and hydrogen partial pressure.

When the catalyst comprises a noble metal on a support, the catalyst may be recovered from the hydrogenation reaction medium, for example, by filtration. The recovered noble metal catalyst may then be recycled and reused in subsequent hydrogenation reaction. It has been shown that the catalyst can be removed from the product mixture using vacuum filtration through a fine-porosity sintered glass filter. In a commercial operation, catalyst filtration can be effected, for example, by using pressure filtration through a sintered metal filter.

The hydrogenation reaction may be further conducted in the presence of a solvent. Examples of suitable solvents include methanol, dichloromethane, acetone, acetonitrile, ethyl acetate, THF, DME, and DMF. Selection of solvent may be based on considerations of solubility, steroid stability, and selectivity. Where the substrate is a 3-alkyl enol ether, a hydroxylic solvent, such as water or an alkanol, may be preferred to protect the enol ether against degradation in the reaction medium, which may otherwise occur to some extent due to atmospheric oxidation. Methanol is a preferred solvent where the substrate is a 3-methyl enol ether.

The hydrogenation reaction is typically mass transfer limited so that reaction rates tend to accelerate with increased hydrogen partial pressure. In operation of the process, hydrogen is supplied to the reactor headspace or sparged subsurface on demand to maintain a total pressure that is sufficient to provide a hydrogen partial pressure at which the hydrogenation reaction can proceed at an acceptable rate. Depending on the intensity of agitation, the reaction may proceed satisfactorily at a hydrogen partial pressure between about 0 and about 100 psig, more typically between about 25 and about 50 psig. With highly intense agitation, reasonable reaction rates may be achieved at hydrogen partial pressures below 20 psig. Depending on the nature of the solvent and the reaction temperature, the solvent vapor pressure may contribute a significant increment to the total pressure. But with adequate agitation, the reaction can be conducted on an economic scale at a total pressure as low as 40 psig, or even 20 psig or less.

Alternatively, a solution of steroid substrate in an appropriate solvent may be caused to flow through a fixed or fluid bed of heterogeneous hydrogenation catalyst, co-currently or countercurrently to a flow of hydrogen. For example, the steroid substrate solution may be introduced at the upper end of a fixed bed or fluid bed contained in a vertical column reaction vessel, and caused to flow downwardly countercurrently to an upward flow of hydrogen gas.

For maximum productivity at a given hydrogen partial pressure, the hydrogenation reaction mass may be subjected to vigorous agitation. However, the reaction can be conducted satisfactorily with more modest agitation, which may require marginally higher hydrogen pressure or marginal extension of batch cycles. Excessively intense agitation may tend to degrade a heterogeneous catalyst.

The hydrogenation reaction is typically conducted at a temperature of from about 0° to about 100° C., preferably at a temperature of from about 25° to about 75° C. Like pressure and agitation, reaction concentration and temperature are interrelated. Reactor payloads can be increased due to increased solubility of steroid substrate in the higher part of the reaction temperature range. Preferably the concentration of 17-ethynyl substrate in the charge solution is at least about 5 wt. %, more preferably at least about 15 wt. %, and still more preferably at least about 20 wt. %. The attainable payload depends also on selection of solvent. Any of the solvents listed above provides satisfactory payloads.

Because hydrolysis of a 3-alkyl enol ether can occur in the presence traces of acid, the hydrogenation may optionally be conducted in the presence of a small concentration of base, typically a nitrogenous base such as triethylamine. In the syntheses described herein in which a 6,7-dehydrogenation is conducted on a 3-alkyl enol ether substrate, formation of impurities in the hydrogenation step proportionally increases impurities in the dehydrogenation step in those embodiments wherein dehydrogention is the solvent medium used for the hydrogenation step without isolation of the steroid intermediate produced by hydrogenation.

In certain preferred embodiments, the hydrogenation reaction is conducted in the presence of an amine inhibitor or a sacrificial reduction target to inhibit over-reduction to the 17-ethyl group. For example, a sacrificial reduction target may be added to a liquid solvent hydrogenation reaction medium to prevent over-reduction of the steroid substrate. It has been found that the addition of an adjuvant such as, for example, an alkene or cycloalkene to the reaction mixture tends to protect the steroid against over-reduction, especially where the steroid substrate is saturated at the C-9/C-11 position. In the absence of an appropriate inhibitor or sacrificial reduction target, it may be difficult to achieve substantially complete conversion of a 9(11)-saturated-17-ethynyl substrate to the 17-vinyl intermediate (or product) without further reduction of a significant fraction of the 17-vinyl to the 17-ethyl group. However, where such inhibitor or target is used, it becomes feasible to consistently and reliably terminate the reaction at the desired end point, i.e., the point during the hydrogenation cycle when the 17-ethynyl is substantially exhausted but loss of 17-vinyl product to 17-ethyl by-product has not yet seriously progressed. Where the reaction mixture includes a sacrificial reduction target, hydrogen is initially preferentially consumed in reduction of the 17-ethynyl to the 17-vinyl but thereafter it is preferentially consumed in reduction of the sacrificial target, thereby averting over-reduction of the steroid to the 17-ethyl species.

Exemplary alkenes suitable for this purpose include α-olefins such as 1-pentene, 1-hexene, 1-octene, etc., and cycloalkenes such as cyclopentene and cyclohexene. Other alkenes, as well as acetylene or other alkynes, may also be used. Preferably, the sacrificial alkene has a vapor pressure low enough so that it does not significantly reduce the hydrogen partial pressure in a reaction conducted at a total pressure up to, for example, 100 psig, but high enough so that, if desired, the alkene and/or its alkane reduction product can be readily removed from the reaction mixture after the reaction is complete by distillation and/or stripping with an inert gas. It will be understood that other alkenes can also be used. In a batch reaction, the alkene may be present in the reactor charge at a preferred concentration equating to an alkene to steroid substrate molar ratio between about 5% and about ≧100%, more preferably between about 10% and about 60%. Excess alkene serves no useful purpose other than to widen the margin of error for detection of the desired reaction end product. Time and energy are consumed in its removal.

Where the reaction is conducted in the presence of a sacrificial reduction target, the reaction end point is conveniently identified by measuring the hydrogen consumption. When hydrogen consumption has exceeded that required for reduction of the 17-ethynyl to 17-vinyl group, or for reduction of a 17-lactenone to 17-spirolactone, it indicates that the conversion of the steroid substrate is substantially complete. To provide a margin of error, hydrogen delivery is preferably continued until consumption of hydrogen reaches perhaps 1.1 to 1.5 times, for some operations more preferably about 1.20 to about 1.35 times, what is theoretically required for the desired reduction reaction. Within these ranges, the desired reduction reaction can ordinarily be deemed complete. Unreacted alkene and alkane reduction product may then be removed from the reaction mixture by distillation, or stripping with an inert gas. For carbonylation of certain substrates run subsequent to hydrogenation in the presence of at least certain α-olefins (e.g., the Pd-catalyzed carbonylation of 17-vinyl testosterone to aldona following hydrogenation of ethisterone to 17-vinyl testosterone in the presence of 1-hexene), it may be desirable to reduce the residual α-olefin to a level that does not significantly interfere with the carbonylation reaction rate.

As described above, over-reduction may also be controlled or prevented by use of an amino inhibitor. Useful inhibitors include pyridine, quinoline, ethylene diamine and lutidine.

While the use of a sacrificial reduction target or amine inhibitor is often preferred in the hydrogenation of a 17-ethynyl steroid substrate that is saturated at C-9(11), it has been unexpectedly discovered that Δ⁹⁽¹¹⁾ steroid substrates possess an inherent resistance to over-reduction. Thus, rigorous endpoint determination and/or use of adjuvants to suppress reduction of the desired 17-vinyl steroid product may not necessarily be required. Instead it has been discovered that, in the case of the latter class of substrates, hydrogen uptake spontaneously stops upon substantially quantitative reduction of the 17-ethynyl to the 17-vinyl group, and a sacrificial reduction target is ordinarily unnecessary for avoiding yield loss to the 17-ethyl species.

Following the hydrogenation reaction, the reaction mass may be filtered for removal of a heterogeneous catalyst. The 17-vinyl product may optionally be recovered from the filtrate by removing the solvent under vacuum. Where 17-vinyl product serves as an intermediate for a subsequent carbonylation, the reaction solution may be directly used in the carbonylation reaction, with or without filtration for removal of the hydrogenation catalyst. In some instances, the hydrogenation catalyst may be effective to promote the carbonylation. Often it is not, but for processing convenience filtration for removal of the hydrogenation catalyst may be deferred until after the carbonlyation step if desired. Where the filtered or unfiltered hydrogenation reaction solution is used directly in the carbonylation reaction, it is desirable to control the concentration of nitrogenous base therein, because the presence of a nitrogenous base can inhibit the carbonylation reaction. In such embodiments of the invention, therefore, the concentration of base in the hydrogenation reaction mixture is preferably limited so a to minimize any adverse effect on the carbonylation. For example, the concentration of triethylamine or other nitrogenous base in the hydrogenation reaction medium may be controlled at a level between about 0.01 and about 100 mole %, preferably less than about 20 mole %, more preferably less than about 10 mole %. When residual nitrogenous base content is sufficient to materially compromise the carbonylation, the inhibiting effect may be overcome by addition of an acidic reducing such as formic in an excess sufficient to both neutralize the base and condition the carbonylation catalyst for the latter reaction. The amine formate salt also functions as an effective reducing agent promoting formation of the carbonylation catalyst.

In an alternative embodiment of the reduction step, the 17-alkynyl may be reduced to the 17-alkenyl group by catalytic transfer reduction, as generally described, e.g., in Johnstone et al., “Metal-Assisted Reactions—Part 10; Rapid, Stereoselective and Specific Catalytic Transfer Reduction of Alkynes to cis-Alkenes,” Tetrahedron, Vol. 37, No. 21, pp. 3667-3670 (1981). In this process, an organic medium comprising the steroid substrate is contacted with a hydrogen donor and a catalyst for the reaction. The donor may typically be a hydrogen source such as cyclohexene, hydrazine, formic acid, a formate salt, phophinic acid, a phophinate salt, phosphorous acid, a phosphite salt, an alcohol or an amine. The catalyst may be a heterogeneous catalyst of the type described above, e.g., Pd/C treated with Pb or Hg to partially poison the catalyst to inhibit further conversion of alkene to alkane. Advantageously, this process may be conducted in a phase transfer system wherein the hydrogen sources is contained in an aqueous medium and a phase transfer catalyst, typically a quaternary ammonium salt such as benzyltrialkyl ammonium halide, serves to transport the hydrogen donor to the phase interface where the heterogeneous catalyst tends to congregate.

In a preferred embodiment of the invention, a 17-ethynyl steroid, e.g., a 17-hydroxy-17-ethynyl steroid, is first reduced to the corresponding 17-vinyl steroid according the hydrogenation procedure described above, after which the 17-vinyl compound is carbonylated. In the case of a 17-hydroxy-17-vinyl intermediate, the resulting steroid product comprises the 17-spirolactone, specifically, the 17-spirobutyrolactone group. Each reaction is conducted substantially as described above.

Although the above description relates to the formation of the 17-spirobutyrolactone by carbonylation of a 17-ethynyl and/or 17-vinyl steroid, it will be understood that higher spirolactones may be formed from higher α-alkenyl or higher α-alkynyl substituents at the 17-position, e.g., α-propenyl, α-propynyl, α-n-butenyl, or α-n-butynyl. Substituted 17-spirolactones can be produced from further substituted 17-alkenyl or 17-alkynyl substituents, or from alkenyl or alkynyl groups that are internally rather than terminally unsaturated.

C. In Situ Carbonylation and Hydrogenation

As previously stated, it has been found that the carbonylation and hydrogenation reactions described above can be conducted in any order or in a single reactor as an in situ carbonylation/hydrogenation to produce a C-17 spirolactone steroid compound.

In an alternative embodiment of the invention, the solvent selected for the hydrogenation is effective for the carbonylation reaction also, in which case the reaction solution produced by the hydrogenation reaction may be used directly in the carbonylation, without first recovering the 17-vinyl intermediate.

In a further alternative embodiment, the process of the present invention comprises simultaneously contacting the steroid substrate with a source of hydrogen, a source of carbon monoxide and a catalyst system effective for reducing the 17-ethynyl group and for carbonylating the resulting derivative in situ to convert the derivative to a 17-spirobutyrolactone structure.

D. Reduction of Lactenone to Spirolactone

Where a mixture of 17-spirolactone and 17-lactenone is produced by direct carbonylation of a 17-ethynyl substrate as described above, the hydrogenation of the lactenone to the spirolactone may be conducted in the manner described in Bull et al., Tetrahedron 1990, 46, 5389; Bull et al., Tetrahedron Lett. 1989, 30, 6907; Alonso et al., J. Org. Chem. 1991, 56, 5567; Cella et al., J. Org. Chem. 1959, 24, 743; and Kamata et al., J. Med. Chem. 1985, 28, 428. This reaction has been found to proceed effectively in the absence of CO. Thus, it is preferred that the steroid mixture be removed from the carbonylation reaction zone prior to hydrogenation for more effective conversion of the lactenone to the spirolactone.

Overall Process

As described above and shown in Reaction Schemes I to VI, certain advantageous embodiments of the present invention are directed to novel processes for the preparation of a compound of Formula XXXII 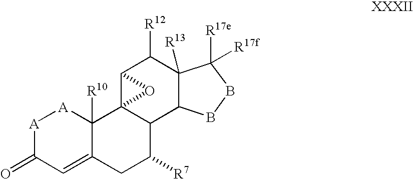
wherein:
R¹⁰, R¹², and R¹³ are independently selected from the group consisting of hydrogen, halo, hydroxy, lower alkyl, lower alkoxy, hydroxyalkyl, alkoxyalkyl, hydroxycarbonyl, cyano, and aryloxy;
-A-A- represents the group —CHR¹—CHR²— or —CR¹═CR²;
where R¹ and R² are independently selected from the group consisting of hydrogen, halo, hydroxy, alkyl, alkoxy, acyl, hydroxyalkyl, alkoxyalkyl, hydroxycarbonyl, alkoxycarbonyl, cyano, and aryloxy, or R¹ and R² together with the carbons of the steroid backbone to which they are attached form a cycloalkyl group;
—B—B— represents the group —CHR⁵—CHR¹⁶—, —CR¹⁵═CR¹⁶— or an α- or β-oriented group: 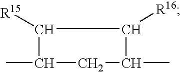
where R¹⁵ and R¹⁶ are independently selected from the group consisting of hydrogen, halo, alkyl, alkoxy, acyl, hydroxyalkyl, alkoxyalkyl, hydroxycarbonyl, alkoxycarbonyl, acyloxyalkyl, cyano, and aryloxy;
or R¹⁵ and R¹⁶, together with the C-15 and C-16 carbons of the steroid nucleus to which they are attached, form a cycloalkylene group; and
R^{17e} and R^{17f} are independently selected from the group consisting of hydrogen, hydroxy, halo, lower alkoxy, acyl, hydroxyalkyl, alkoxyalkyl, hydroxycarbonylalkyl, alkoxycarbonylalkyl, acyloxyalkyl, cyano, and aryloxy, or R^{17e} and R^{17f} together comprise a carbocyclic or heterocyclic ring structure, or R^{17e} or R^{17f} together with R¹⁵ or R¹⁶ comprise a carbocyclic or heterocyclic ring structure fused to the pentacyclic D ring.

In a particularly preferred embodiment, the processes of the invention are implemented for the preparation of methyl hydrogen 9(11)α-epoxy-17α-hydroxy-3-oxopregn-4-ene-7α,21-dicarboxylate, γ-lactone (i.e., eplerenone or epoxymexrenone). Each of these process schemes involves the carbonylation reaction of the invention, and most also involve the hydrogenation of a 17-ethynyl to a 17-vinyl group. Many of these reactions start with the substrate of Formula XX: 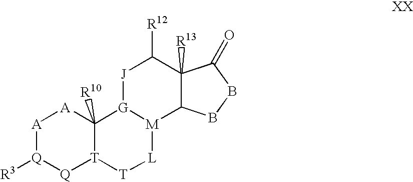
wherein
R³ is selected from the group consisting of hydrogen, hydroxy, alkoxy, hydroxyalkyl, alkoxyalkyl and hydroxycarbonyl, di(hydrocarbyl)amino, di(substituted hydrocarbyl)amino and N-heterocyclyl;
R¹⁰, R¹², and R¹³ are independently selected from the group consisting of hydrogen, halo, hydroxy, lower alkyl, lower alkoxy, hydroxyalkyl, alkoxyalkyl, hydroxycarbonyl, cyano, and aryloxy;
-A-A- represents the group —CHR¹—CHR²— or —CR¹═CR²—;
R¹ and R² are independently selected from the group consisting of hydrogen, halo, hydroxy, alkyl, alkoxy, acyl, hydroxyalkyl, alkoxyalkyl, hydroxycarbonyl, alkoxycarbonyl, cyano, and aryloxy, or R¹ and R² together with the carbons of the steroid backbone to which they are attached form a cycloalkyl group;
—B—B— represents the group —CHR¹⁵—CHR¹⁶—, —CR¹⁵═CR¹⁶— or an α- or β- oriented group: 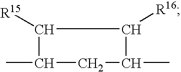
where R¹⁵ and R¹⁶ are independently selected from the group consisting of hydrogen, halo, alkyl, alkoxy, acyl, hydroxyalkyl, alkoxyalkyl, hydroxycarbonyl, alkoxycarbonyl, acyloxyalkyl, cyano, and aryloxy; or R¹⁵ and R¹⁶ together with the C(15) and C(16) carbons of the steroid nucleus to which they are attached, form a cycloalkylene group, such as, e.g., cyclopropylene;
-G-J- represents the group 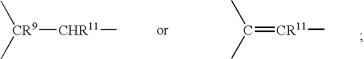
where R⁹ and R¹¹ are independently selected from the group consisting of hydrogen, halo, alkyl, alkoxy, acyl, hydroxyalkyl, alkoxyalkyl, hydroxycarbonyl, alkoxycarbonyl, acyloxyalkyl, cyano and aryloxy; or R⁹ and R¹¹ together with the C-9 and C-11 carbons of the steroid nucleus to which they are attached, form an epoxy group;
-Q- Q- represents the group 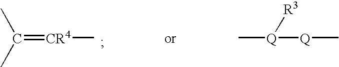
together represent the group 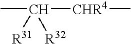
wherein R³¹ and R³² are independently selected from the group consisting of hydroxy and alkoxy, or R³¹, R³² and the C-3 carbon of the steroid nucleus to which they are attached for the group 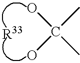
where R³³ ia alkylene.
-T-T- represents the group 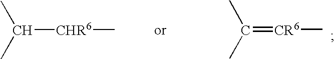
where R⁶ is selected from the group consisting of hydrogen, halo, alkyl, alkoxy, acyl, hydroxyalkyl, alkoxyalkyl, hydroxycarbonyl, alkoxycarbonyl, acyloxyalkyl, cyano and aryloxy;
-L-M- represents the group 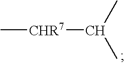
where R⁷ is selected from the group consisting of hydrogen, halo, alkyl, cycloalkyl, alkoxy, acyl, hydroxyalkyl, alkoxyalkyl, hydroxycarbonyl, alkoxycarbonyl, acyloxyalkyl, cyano, aryloxy, thioacetyl, furyl and substituted furyl; or R⁵ and R⁶, together with the C-5 and C-6 carbons of the steroid nucleus to which they are attached, form a cycloalkylene group.

In various preferred embodiments, the compound of Formula XX is a compound of Formula XXA: 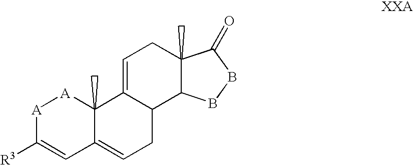
wherein
-A-A- represents the group —CH₂—CH₂— or —CH═CH—;
R³ is lower alkoxy; and
—B—B— represents the group —CH₂—CH₂— the cyclo propylene group 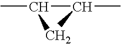
or an α- or β-oriented group:

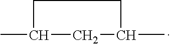

A particularly preferred starting substrate corresponds to the Formula: 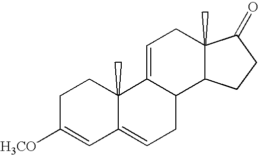
referred to herein as 112DM, which is especially suited as a starting material for the preparation of epoxymexrenone. The 2DM substrate is advantageous in comprising a 3-methyl enol ether that is useful for later introduction of 6,7-unsaturation, which in turn ultimately enables formation of the 7α-methoxycarbonyl moiety of epoxymexrenone.

Various alternative processes embodying the carbonylation and/or hydrogenation step(s) of the present invention are described herein for the preparation of epoxymexrenone starting with 2DM, with the formation of a number of different intermediates derived from 2DM. It will be understood, however, that the processes as described are applicable to other substrates and intermediates comprising alternative substituents within the generic scope of the definitions set forth herein for Formulae 1502, 1503, 2502 and 2503.

Processes utilizing 2DM begin with the 17-ethynylation thereof. For this purpose, 2DM may be contacted with acetylene in the presence of a strong base. For example, 2DM or a similar substrate may be contacted with acetylene gas in the presence of an alkali metal alkoxide such as, for example potassium t-butoxide, or by reaction with acetylene salts as described, e.g., in Sondheimer et al. U.S. Pat. No. 2,888,471; Velluz et al., J. Am. Chem. Soc. 1958, 80, 2726, Teutsh et al. U.S. Pat. No. 4,168,306 and Van Rheenen et al. J. Org. Chem., 1979, 44, 1582.

More particularly, ethynylation can be conducted in accordance with the method described by Colton et al., J. Am. Chem. Soc., Vol. 59 (1959), pp. 1123-1127 wherein an ethynylation medium is prepared by passing a slow stream of acetylene over a stirred solution of an alkali metal alkoxide in the corresponding alcohol, e.g., K t-amylate in t-amyl alcohol, and another organic solvent such as a dialkyl ether, preferably in the cold, e.g., −10° to 10° C. Conveniently, the medium may comprise approximately equal volumes of alcohol and dialkyl ether, and contain 2 to 75 gpl., more typically 10 to 40 gpl, alkali metal. After the medium has become saturated with acetylene gas, the steroid substrate is added, preferably in a proportion limited so as to maintain a stoichiometeric excess of alkali metal alkoxide. Preferably, addition of acetylene is continued in the cold for a period, e.g., 2 to 6 hours, after which the reaction mixture may be warmed moderately, e.g., to room temperature, to complete the reaction. Reaction may take 12 to 24 hours. Alternatively, the ethynylation may be conducted in the manner described in Marshall et al., J. Biol. Chem., 1957, pp. 340-350 wherein a slow stream of acetylene is introduced into a dilute solution of steroid substrate, e.g., 5 to 20 gpl substrate in a solvent such as 3:2 benzene-anhydrous ether. Thereafter, a solution of alkali metal in alcohol, e.g., 1 to 5 gpl potassium in t-amyl alcohol, is added to the solvent medium rapidly under agitation. Addition of acetylene is continued for a period of 2-10 hours. At the end of the reaction period, the reaction solution is flushed with nitrogen and diluted with solvent, typically to increase the volume 50% to 200%, after which the diluted reaction solution is contacted with a weak acid to quench the base. For example, a saturated solution of ammonium chloride solution may be added in progressive proportions ultimately roughly equivalent in volume to the diluted reaction solution. The aqueous phase may be extracted with organic solvent, e.g., benzene/ether to recovered residual ethynyl steroid product therefrom.

Since the cyclic and open-chain forms, that is to say lactones and 17β-hydroxy-21-carboxylic acids and their salts, respectively, are so closely related to each other that the latter may be considered merely as a hydrated form of the former, there is to be understood hereinbefore and hereinafter, unless specifically stated otherwise, both in end products of the Formula XXXII and in starting materials and intermediates of analogous structure, in each case all the mentioned forms together.

Thus, the product of the carbonylation may correspond to the formula: 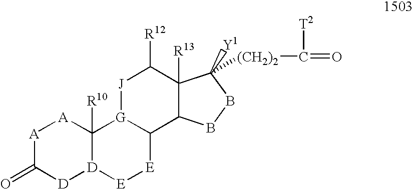
wherein
Y¹ and Y² together represent the oxygen bridge —— or Y¹ represents hydroxy and Y² represents hydroxy alkoxy or O⁻M⁽⁺⁾, is a monovalent cation or the combination of a polyvalent cation and another anion. It will be understood that the another anion can have the same construction as the steroid residue of formula 1503, or can comprise a different anion such as Cl⁻, SO₄, H₂PO₄, HPO₄, H₂PO₄⁻³, other mineral anion or other organic anion.

Reaction Scheme I begins with a compound of Formula XX, as defined above. The compound of Formula XX is alkynylated, forming a compound of Formula XXI: 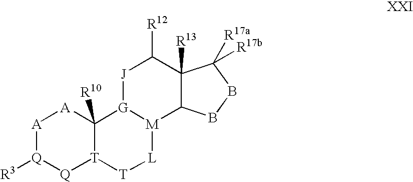
wherein
R³ is selected from the group consisting of hydrogen, hydroxy, alkoxy, hydroxyalkyl, alkoxyalkyl and hydroxycarbonyl, dihydrocarbylamino, di(substituted hydrocarbyl)amino, and N-heterocyclyl;
R¹⁰, R¹², and R¹³ are independently selected from the group consisting of hydrogen, halo, hydroxy, lower alkyl, lower alkoxy, hydroxyalkyl, alkoxyalkyl, hydroxycarbonyl, cyano, and aryloxy;
R^{17a} and R^{17b} are independently selected from the group consisting of hydroxy, protected hydroxy, and alkynyl;
-A-A- represents the group —CHR¹—CHR²— or —CR¹═CR²;
where R¹ and R² are independently selected from the group consisting of hydrogen, halo, hydroxy, alkyl, alkoxy, acyl, hydroxyalkyl, alkoxyalkyl, hydroxycarbonyl, alkoxycarbonyl, cyano, and aryloxy, or R¹ and R² together with the carbons of the steroid backbone to which they are attached form a cycloalkyl group;
—B—B— represents the group —CHR¹⁵—CHR¹⁶—, —CR¹⁵═CR¹⁶ or an α- or β-oriented group: 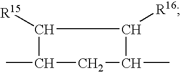
where R¹⁵ and R¹⁶ are independently selected from the group consisting of hydrogen, halo, alkyl, alkoxy, acyl, hydroxyalkyl, alkoxyalkyl, hydroxycarbonyl, alkoxycarbonyl, acyloxyalkyl, cyano, and aryloxy or R¹⁵ and R¹⁶, together with the C-15 and C-16 carbons of the steroid nucleus to which they are attached, form a cycloalkylene group, (e.g., cyclopropylene).
-G-J- represents the group 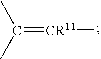
where R¹¹ is selected from the group consisting of hydrogen, halo, alkyl, alkoxy, acyl, hydroxyalkyl, alkoxyalkyl, hydroxycarbonyl, alkoxycarbonyl, acyloxyalkyl, cyano and aryloxy;
-Q-Q- represents the group 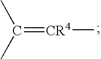
where R⁴ is selected from the group consisting of hydrogen, halo, alkyl, alkoxy, acyl, hydroxyalkyl, alkoxyalkyl, hydroxycarbonyl, alkoxycarbonyl, acyloxyalkyl, cyano and aryloxy;
-T-T- represents the group 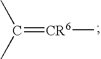
where R⁶ is selected from the group consisting of hydrogen, halo, alkyl, alkoxy, acyl, hydroxyalkyl, alkoxyalkyl, hydroxycarbonyl, alkoxycarbonyl, acyloxyalkyl, cyano and aryloxy; and
-L-M- represents the group 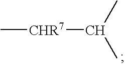
where R⁷ is selected from the group consisting of hydrogen, halo, alkyl, cycloalkyl, alkoxy, acyl, hydroxyalkyl, alkoxyalkyl, hydroxycarbonyl, alkoxycarbonyl, acyloxyalkyl, cyano, aryloxy, acetylthio, furyl and substituted furyl;
or R⁶ and R⁷ and the C-6 and C-7 carbons of the steroid nucleus to which they are attached form a cycloaklylene group (e.g., cyclopropylene).

In one preferred embodiment of the first step of Scheme I, the compound of Formula XX is a compound of Formula XXA: 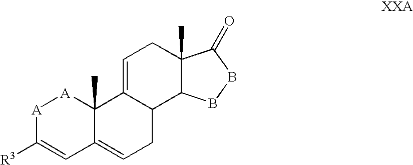
wherein
-A-A- represents the group —CH₂—CH₂— or —CH═CH—;
R³ is lower alkoxy; and
—B—B— represents the group —CH₂—CH₂— or an α- or β-oriented group: 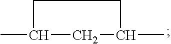
and the compound of Formula XXI is a compound of Formula XXIA: 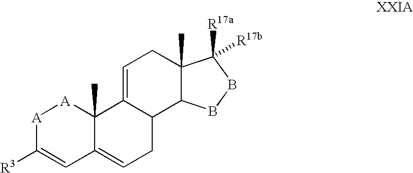
wherein
-A-A- represents the group —CH₂—CH₂— or —CH═CH—;
R³ is lower alkoxy;
—B—B— represents the group —CH₂—CH₂—, —CR¹⁵═CR¹⁶— or an α- or β-oriented group: 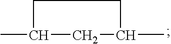
R^{17a} is hydroxy or protected hydroxy; and
R^{17b} is alkynyl.

In a particularly preferred embodiment of the second step of Scheme I, the compound of Formula XX is 2DM: 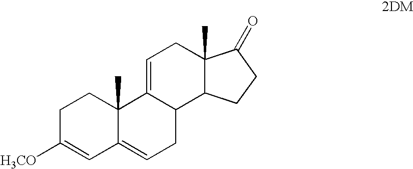
and the compound of Formula XXI is ethynyl 2DM:

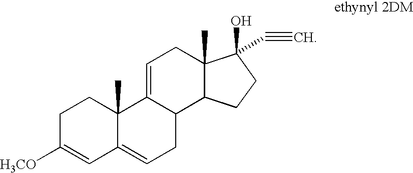

In the second step of Scheme I, the compound of Formula XXI is semi-hydrogenated, preferably by contact with a source of hydrogen in accordance with the hydrogenation process described herein, to produce a compound of Formula XXII: 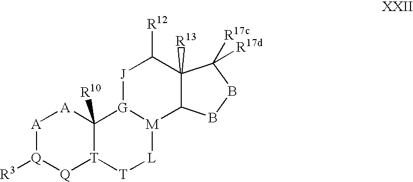
wherein R³, R¹⁰, R¹², R¹³, -A-A-, —B—B—, -G-J-, -Q-Q-, -T-T-, and -L-M- are as defined above for Formula XXI; and R^{17c} and R^{17d} are independently selected from the group consisting of hydroxy, protected hydroxy, and alkenyl.

In one preferred embodiment of the second step of Scheme I, the compound of Formula XXI is a compound of Formula XXIA as shown above, and the compound of Formula XXII is a compound of Formula XXIIA: 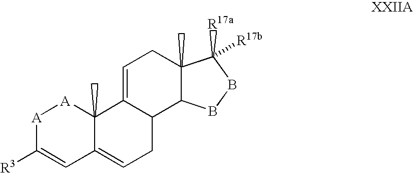
wherein
-A-A- represents the group —CH₂—CH₂— or —CH═CH—;
R³ is lower alkoxy;
—B—B— represents the group —CH₂—CH₂— or an α- or β-oriented group: 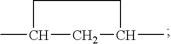
R^{17a} is hydroxy or protected hydroxy; and
R^{17b} is alkenyl.

In a particularly preferred embodiment of the second step of Scheme I, the compound of Formula XXI is ethynyl 2DM, as shown above, and the compound of Formula XXII is a compound of Formula A as shown in Table 1 above, and also called “vinyl 2DM” herein.

In the third step of Scheme I, the compound of Formula XXII is carbonylated as described herein to produce a compound of Formula XXIII: 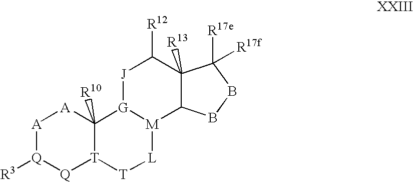
wherein R³, R¹⁰, R¹², R¹³, -A-A-, —B—B—, -G-J-, -Q-Q-, -T-T-, and -L-M- are as defined above for Formula XXI; and R^{17e} and R^{17f} are independently selected from the group consisting of hydroxy, hydroxycarbonylalkyl, and alkoxycarbonylalkyl, or R^{17e} and R^{17f} together with the carbon to which they are attached comprise a heterocyclic ring structure.

In one preferred embodiment of the third step of Scheme I, the compound of Formula XXII is a compound of Formula XXIIA as shown above, and the compound of Formula XXIII is a compound of Formula XXIIIA: 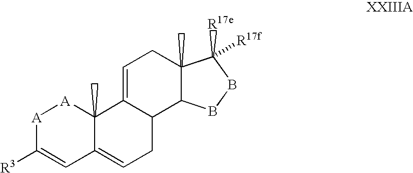
wherein
-A-A- represents the group —CH₂—CH₂— or —CH═CH—;
R³ is lower alkoxy;
—B—B— represents the group —CH₂—CH₂— or an α- or β-oriented group: 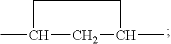
   and

R^{17e} is hydroxy and R^{17f} is hydroxycarbonylalkyl, or R^{17e} and R^{17f} together with the carbon to which they are attached form a lactone ring.

In a particularly preferred embodiment of the third step of Scheme I, the compound of Formula XXII is vinyl 2DM, as shown above, and the compound of Formula XXIII is (17α)-pregna-3,5,9(11)-triene-21-carboxylic acid, γ-lactone: 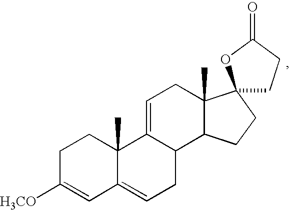
also called “spiro 2DM.”

In the fourth step of Scheme I, the compound of Formula XXIII is oxidized (i.e., dehydrogenated), preferably by contact with an oxidizing agent such as DDQ or chloranil in the presence of water, to form a compound of Formula XXVIII: 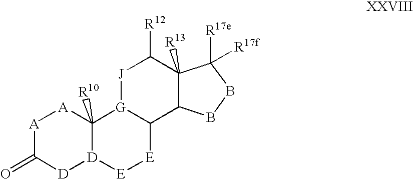
wherein
R¹⁰, R¹², R¹³, -A-A-, and —B—B— are as defined above for Formula XXI; R^{17e} and R^{17f} are as defined above for Formula XXIII; and
-D-D- represents the group 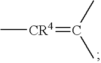
where R⁴ is selected from the group consisting of hydrogen, halo, alkyl, alkoxy, acyl, hydroxyalkyl, alkoxyalkyl, hydroxycarbonyl, alkoxycarbonyl, acyloxyalkyl, cyano and aryloxy or R⁴ and R⁵ together with the carbons of the steroid backbone to which they are attached form a cycloalkyl group;
-G-J- represents the group 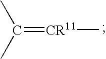
where R¹¹ is selected from the group consisting of hydrogen, hydroxy, protected hydroxy, halo, alkyl, alkoxy, acyl, hydroxyalkyl, alkoxyalkyl, hydroxycarbonyl, alkoxycarbonyl, acyloxyalkyl, cyano and aryloxy or R⁹ and R¹¹ together form an epoxy group; and
-E-E- represents the group —CR⁶═CR⁷—;
where R⁶ is selected from the group consisting of hydrogen, halo, alkyl, alkoxy, acyl, hydroxyalkyl, alkoxyalkyl, hydroxycarbonyl, alkoxycarbonyl, acyloxyalkyl, cyano and aryloxy; and
R⁷ is selected from the group consisting of hydrogen, halo, alkyl, cycloalkyl, alkoxy, acyl, hydroxyalkyl, alkoxyalkyl, hydroxycarbonyl, alkoxycarbonyl, acyloxyalkyl, cyano, aryloxy, acetylthio, furyl and substituted furyl.

In one preferred embodiment of the fourth step of Scheme I, the compound of Formula XXIII is a compound of Formula XXIIIA, as shown above, and the compound of Formula XXVIII is a compound of Formula XXVIIIA: 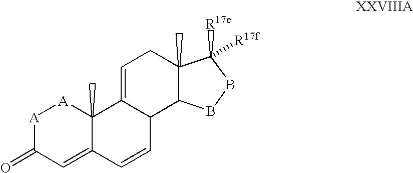
wherein
-A-A- represents the group —CH₂—CH₂— or —CH═CH—;
—B—B— represents the group —CH₂—CH₂— or an α- or β-oriented group: 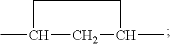
R⁷ is selected from the group consisting of hydrogen, furyl, and alkylfuryl; and
R^{17e} is hydroxy and R^{17f} is hydroxycarbonylalkyl, or R^{17e} and R^{17f} together with the carbon to which they are attached form a lactone ring.

In a particularly preferred embodiment of the fourth step of Scheme I, the compound of Formula XXIII is spiro 2DM, shown above, and the compound of Formula XXVIII is Δ⁹⁽¹¹⁾-canrenone:

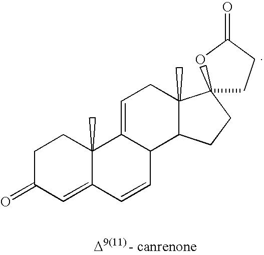

In the fifth step of Scheme I, the compound of Formula XXVIII is contacted with an alkyl furan in the presence of a Lewis acid, a proton acid with a pKₐ of less than about 5, or a salt of a secondary amine of the formula 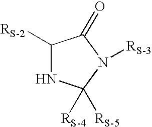
where:
R_{S-2} is —H, C₁-C₄ alkyl, phenyl, and benzyl;
R^{S-3} is —H, C₁-C₄ alkyl;
R^{S-4} is —H, C₁-C₄ alkyl, phenyl;
R^{S-5} is —H, C₁-C₄ alkyl, phenyl; and 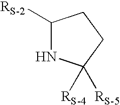
where:
R^{S-2} is —H, C₁-C₄ alkyl, phenyl, and benzyl;
R^{S-4} is —H, C₁-C₄ alkyl, phenyl;
R^{S-5} is —H, C₁-C₄ alkyl, phenyl;
with an acid of pKa of less than about 2, to form a compound of Formula XXIX: 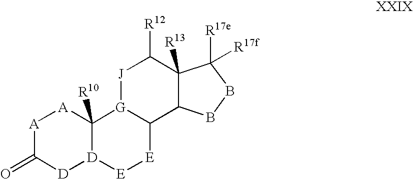
wherein R¹⁰, R¹², R¹³, -A-A-, and —B—B— are as defined above for Formula XXI; R^{17e} and R^{17f} are as defined above for Formula XXIII; and -D-D-, -G-J-, and -E-E- are as defined above for Formula XXVIII.

Preferably, the compound of Formula XXVIII is contacted with an alkylfuran in the presence of a Lewis acid. The Lewis acid must be electrophilic enough to complex with the Δ4,6-3-keto steroid of Formula XXVIII, but not so electrophilic that it complexes with the nucleophilic alkylfuran, as is known to those skilled in the art. Further, it is preferred that the Lewis acid be used in the presence of an alcohol selected from the group consisting of C₁-C₃ alcohols, ethylene glycol, 1,2- or 1,3-propylene glycol, 2,2-dimethyl- or 2,2-dimethyl-1,3-propylene glycol and phenol. It is more preferred that the alcohol be a C₁-C₃ alcohol or mixture thereof. Useful Lewis acids include those selected from the group consisting of BX₃, AlX₃, SnX₂, SnX₄, SiX₄, MgX₂, ZnX₂, TiX₄, Rh(acac) (CH₂CH₂)₂(2,2′-bis(diphenyphosphino)-1,1′-binaphthyl), Rh(CH₃—CN)₂(cyclooctadiene) (BF₄), Rh(acac) (CH₂CH₂)₂(dppb), LiClO₄, K10 Montmorillonite clay, Yb(OTf)₃, LiCo(B₉C₂H₁₁)₂, PdX₂, CrX3, FeX₃, CoX₃, NiX₂, SbX₅, InX₃, Sc(OTf)₃, (phenyl)₃C⁺X⁻, R₃SiX, Pd(CH₃O—CO—O—)₂; BF₃-diethyletherate complex, BF₃-acetic acid complex, BF₃-methyl-t-butyl ether complex, BF₃-di-n-butyletherate complex; BF₃-dimethyletherate complex; BF₃-dimethylsufide complex; BF₃-phenol complex; BF₃-phosphoric acid complex; and BF₃-tetrahydrofuran complex; where R is C₁-C₄ alkyl or phenyl; and where X is selected from the group consisting of F⁻, Cl⁻, Br⁻, I⁻, —O—SO₂CF₃⁻, PF₆⁻, BF₄⁻, and ClO₄⁻.

It is preferred that the Lewis acid is selected from the group consisting of BF₃, BF₃-diethyletherate complex, BF₃-acetic acid complex, BF₃-methyl-t-butyl ether complex, BF₃-di-n-butyletherate complex, BF₃-dimethyletherate complex, BF₃-dimethylsulfide complex, BF₃-phenol complex, BF₃-phosphoric acid complex, and BF₃-tetrahydrofuran complex. It is more preferred that the Lewis acid is BF₃-diethyletherate. It is even more preferred that the BF₃-diethyletherate is used in the presence of C₁-C₃ alcohol and still more preferred is the use of the BF₃-diethyletherate in the presence of C₂ alcohol. Useful acids with a pKₐ of less than about 5 are selected from the group consisting of formic acid, acetic acid, propionic acid, benzoic acid, hydrofluoric acid, fluoroboric acid, p-toluenesulfonic acid, methanesulfonic acid, benzenesulfonic acid, trifluoromethanesulfonic acid, perchloric acid, trifluoroacetic and trichloroacetic. It is preferred that the acid with a pKₐ of less than about 5 is acetic acid.

When performing the transformation of the compound of Formula XXVIII to a compound of Formula XXIX at least one equivalent of the alkyl furan should be used; it is preferable to use from one to two equivalents. Use of additional reagent is not a problem, but rather a waste of compound.

The reaction can be carried out in a variety of solvents, such as in a solvent/solvent mixture selected from the group consisting of C₁-C₆ alcohols, a solvent mixture of C₁-C₆ alcohols, and a solvent selected from the group consisting of acetonitrile, nitromethane, toluene, methylene chloride and acetic acid. One factor to be considered in selecting a Lewis acid and solvent is the acid sensitivity of the 7α-substituted steroid of Formula XXIX. The reaction must be performed with a Lewis acid and in a solvent where the product is stable as is known to those skilled in the art. It is preferred that the solvent be a protic solvent, one that has a pKₐ of less than about 19.

The reaction can be performed in a temperature range of from about −78° to about 60° C.; preferably in a temperature range of from about −40′ to about −15° C. It is more preferred to perform the reaction at about −20° C. The reaction normally will take from a few hours to a day depending on the number of equivalents used and the reaction temperature.

Rather than carrying the 7α-substituted steroid intermediate of Formula XXIX on to the next step in situ, it is preferred to isolate and purify the 7α-substituted steroid intermediate before performing the next step. The preferred method of purification of the 7α-substituted steroid intermediate is by crystallization. The process for purifying the 7α-substituted steroid intermediate comprises crystallizing the 7α-substituted steroid intermediate, which contains greater than 5% of the 7β-isomer from a solvent selected from the group consisting of ethyl acetate, n-propyl acetate, and butyl acetate. It is preferred to obtain the 7α-substituted steroid intermediate in greater than 99.8% isomeric purity and ist is preferred that the crystallization solvent is n-propyl acetate. Crystallization co-solvents may be used.

In one preferred embodiment of the fifth step of Scheme I, the compound of Formula XXVIII is a compound of Formula XXVIIIA, as shown above, and the compound of Formula XXIX is a compound of Formula XXIXA: 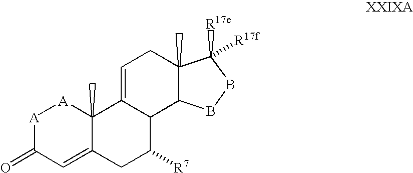
wherein
-A-A- represents the group —CH₂—CH₂— or —CH═CH—;
—B—B— represents the group —CH₂—CH₂— or an α- or β-oriented group: 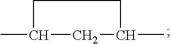
R⁷ is selected from the group consisting of hydrogen, furyl, and alkylfuryl; or
—B—B— constitutes 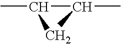
R^{17e} is hydroxy and R^{17f} is hydroxycarbonylalkyl, or R^{17e} and R^{17f} together with the carbon to which they are attached form a lactone ring.

In a particularly preferred embodiment of the fifth step of Scheme I, the compound of Formula XXVIII is Δ⁹⁽¹¹⁾-canrenone, as shown above, and the compound of Formula XXIX is:

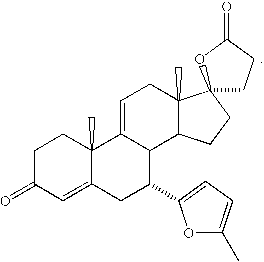

In step six of Scheme I, the 7α-furyl intermediate compound of Formula XXIX as shown above is converted to the 7α-hydroxycarbonyl intermediate compound of Formula XXX: 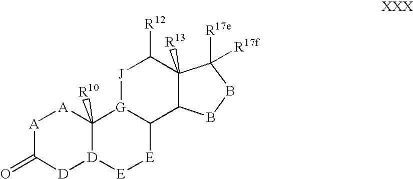
wherein R¹⁰, R¹², R¹³, -A-A-, and —B—B— are as defined above for Formula XXI; R^{17e} and R^{17f} are as defined above for Formula XXIII; and -D-D-, -G-J-, and -E-E- are as defined above for Formula XXVIII.

The conversion of the 7α-furyl intermediate compound of Formula XXIX to the 7α-hydroxycarbonyl intermediate compound of Formula XXX is done by an oxidative process which comprises contacting the compound of Formula XXIX with an agent selected from the group consisting of a halogenating agent in the presence of water and a base whose conjugate acid has a pKa of greater than about 8; an oxygen donating agent; electrochemical oxidation; a quinone in the presence of water; or nonquinone oxidants, to form a cis-enedione intermediate compound of Formula XXIX-1-cis: 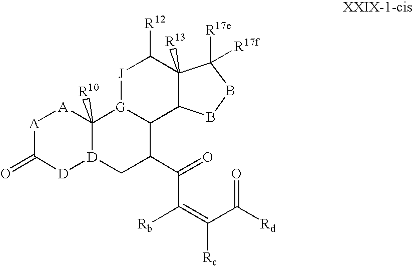
wherein R¹⁰, R¹², R¹³, -A-A-, and —B—B— are as defined above for Formula XXI; R^{17e} and R^{17f} are as defined above for Formula XXIII; -D-D-, -G-J-, and -E-E- are as defined above for Formula XXVIII; and R_{b}, R_{c} and R_{d} are independently selected from the group consisting of hydrogen and alkyl.

The cis-enedione can be transformed to the corresponding trans-enedione of Formula XXIX-1-trans 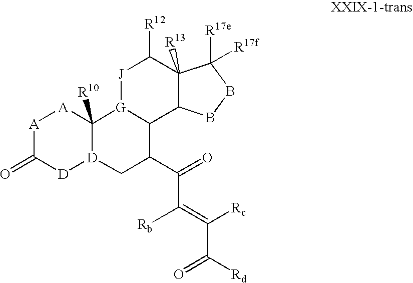
wherein R¹⁰, R¹², R¹³, -A-A-, and —B—B— are as defined above for Formula XXI; R^{17e} and R^{17f} are as defined above for Formula XXIII; -D-D- and -G-J-, and -E-E- are as defined above for Formula XXVIII; and R_{b}, R_{c}, and R_{d} are as defined above for Formula XXIX-1-cis.

It is preferred that the agent be a halogenating agent. Useful halogenating agents include those selected from the group consisting of dibromodimethylhydantoin, dichlorodimethylhydantoin, diiododimethylhydantoin, N-chlorosuccinamide, N-bromosuccinamide, N-iodosuccinamide, trichloroisocyanuric acid, t-butylhypochlorite and 3-bromo-1-chloro-5,5-dimethylhydantoin; it is preferred that the halogenating is dibromodimethylhydantoin. When using a halogenating agent, the amount used should be at least one equivalent of the halogenating agent; preferably from about 1.0 to about 1.05 equivalents of the halogenating agent are used. It is more preferred that the amount of halogenating agent be about 1.01 equivalents. The reason is that one equivalent is required to complete the reaction but any excess needs to be quenched. Suitable quenching agents include bisulfite, isobutylvinyl ether, 2-methylfuran and hypophosphorous acid. Useful oxygen donating agents include those selected from the group consisting of: a peracid, singlet oxygen followed by either phosphite or thiourea, triplet oxygen, hydrogen peroxide with a ketone selected from the group consisting of Q₄-CO-Q₅ where Q₄ and Q₅ are the same or different and are: C₁-C₄ alkyl optionally substituted with 1 thru 9 —Cl or —F, and where the Q₄ and Q₅ are taken together with the attached carbon atom to form a cyclic ketone of 5 thru 7 members and ketones of the formula: 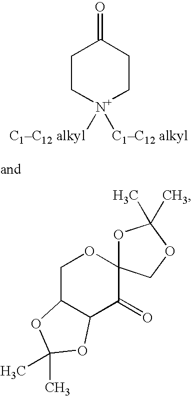
hydrogen peroxide in combination with methyltrioxorhenium, trichloroacetonitrile/hydrogen peroxide, trichloroacetamide/hydrogen peroxide, DDQ/water, p-chloranil/water, phenyl-C(CH₃)₂—O—OH or an alkylhydroperoxide in combination with a metal containing activator, where alkyl is from C₄-C₁₀ alkyl and metal containing activator is selected from the group consisting of Ti(isopropoxide)₄, peroxotungstophosphate, VO(acetylacetonate)₂ and MO hexacarbonyl. It is preferred that the oxygen donating agent is a peracid. Useful peracids include those selected from the group consisting of: (a) perbenzoic acid optionally substituted with 1 or 2 —Cl or —NO₂, (b) percarboxylic acids of the formula Cₙ₂(Q₆)2ₙ₂₊₁—CO₃H where n₂ is 1 thru 4 and Q₆ is —H, —Cl or —F, (c) perphthalic acid and (d) magnesium peroxyphthalate. An excess oxygen donating agent present must also be quenched as was done for the halogenating agents. Base is required to neutralize the acid produced during the formation of the intermediate compound of Formula XXIX-1-cis. Useful bases include those selected from the group consisting of acetate, bicarbonate, carbonate, propionate, benzoate, dibasic phosphate and borate; it is more preferred that the base be acetate. For example, when the halogenating agent is dibromodimethylhydantoin, hydrobromic acid is produced. Hence, one equivalent of base per equivalent of acid produced is required. In practice, a slight excess is used, about 1.5 equivalents. Suitable solvents for this reaction are those which are water miscible and which dissolves both the 7α-substituted steroid (II) and the halogenating agent or oxygen donating agent. Acetone and THF are preferred solvents. The reaction is performed at room temperature, about 20° to about 25° C. The reaction takes a few hours depending on the reactivity of the oxygenating donating agent or halogenating agent. When formed, the compound of Formula XXIX-1-cis does not have to be isolated and purified, but rather can be used in subsequent transformations “as is” or in situ. Other oxidants useful for transformation of the 7α-substituted steroid to the cis-enedione include quinones. The 7α-substituted steroid is contacted with a stoichiometric amount of quinone and at least a stoichiometric amount of quinone and at least a stoichiometric amount of water in a water-miscible organic solvent. The contacting is preferably done at around room temperature. In addition, the oxidation can be accomplished by electrochemistry. The electrochemical oxidation is accomplished by contacting the 7α-substituted steroid with a sub-stoichiometric amount of quinone (preferably DDQ) and at least a stoichiometric amount of water in an electrochemical cell using standard electrochemical techniques such as are described in U.S. Pat. No. 4,270,994. Finally, the oxidation can be accomplished with non-quinone agents which include, manganic acetate, potassium permanganate, ceric ammonium nitrate, iodosobenzene, iodobenzenediacetate, iodobenzenebistrifluoroacetate, chromic acid (“Jones reagent”), and lead tetraacetate. These reactions are typically run in aqueous acetone as solvent at around room temperature (20°-25° C.), although many water-miscible organic co-solvents can be used in place of acetone. Other oxidizing agents that effect this transformation include hydrogen peroxide or an organic hydroperoxide (listed elsewhere) in combination with a metal catalyst such as methyltrioxorhenium, palladium acetate, ruthenium trichloride, or ruthenium tetroxide. These reactions can be run in any solvent in which the 7α-substituted steroid is soluble such as methylene chloride, acetone, etc. The reactions involving ruthenium catalysts are preferably run in aqueous acetonitrile.

The cis-enedione can be transformed to the corresponding trans-enedione (Formula XXIX-1-trans) or it can be converted to the peroxy compound (Formula XXIX-1-OOH): 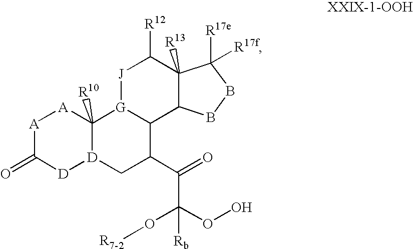
wherein R¹⁰, R¹², R¹³, -A-A-, and —B—B— are as defined above for Formula XXI; R^{17e} and R^{17f} are as defined above for Formula XXIII; -D-D-, -G-J-, and -E-E- are as defined above for Formula XXVIII; R_{b} is as defined above for Formula XXIX-1-cis; and R7-2 is hydrogen or C1-C4 alkyl optionally substituted with one or two hydroxyl groups; the hydroxy compound (Formula XXIX-1-OH): 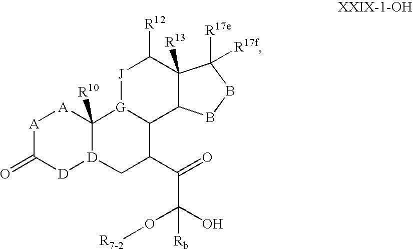
wherein R¹⁰, R¹², R¹³, -A-A-, and —B—B— are as defined above for Formula XXI; R^{17e} and R^{17f} are as defined above for Formula XXIII; -D-D-, -G-J-, and -E-E- are as defined above for Formula XXVIII; R_{b} is as defined above for Formula XXIX-1-cis; and R7-2 is hydrogen or C₁-C₄ alkyl optionally substituted with one or two hydroxyl groups; the biscarbonyl compound (Formula XXIX-2): 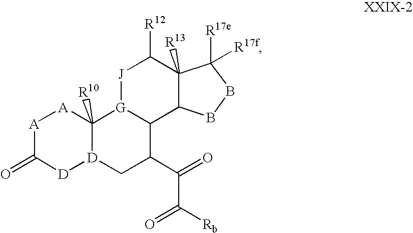
wherein R¹⁰, R¹², R¹³, -A-A-, and —B—B— are as defined above for Formula XXI; R^{17e} and R^{17f} are as defined above for Formula XXIII; -D-D-, -G-J-, and -E-E- are as defined above for Formula XXVIII; R_{b} is as defined above for Formula XXIX-1-cis; and R7-2 is hydrogen or C₁-C₄ alkyl optionally substituted with one or two hydroxyl groups; or the carboxylic acid (Formula XXX) or a mixture thereof. When the term carboxylic acid (XXX) is used, it refers to and includes the pharmaceutically-acceptable salts thereof. These will include the sodium, potassium, lithium, magnesium, tetrabutylammonium and the carboxylic acid salts with DBU, tetramethylquanidien, triethylamine and others. The identity of the particular cation is not important since eventually it is lost when forming an acid which ultimately is converted to the methyl ester (XXXI) and eplerenone (XXXII) which requires a methyl ester at the 7α-position. It is preferable to convert the cis-enedione to the corresponding trans-enedione rather than convert the cis-enedione to a mixture of peroxy, hydroxy and biscarbonyl compounds.

When the cis-enedione is transformed to the corresponding trans-enedione, the cis-enedione is contacted with an isomerization catalyst which can be either a chemical agent including: (a) a strong acid of pKₐ of less than about 2; (b) a tertiary amine whose conjugate acid has a pKₐ greater than about 8 and (c) salt of a tertiary amine whose conjugate acid has a pKₐ greater than about 8, (d) 12, (e) (C₁-C₄)₃P, (f) (phenyl)₃P, or a physical agent such as (g) heating to about 80° C.

It is preferred that the isomerization catalyst be a strong acid of pKₐ of less than about 2. When the isomerization catalyst is a strong acid of pKₐ of less than about 2, useful strong acids of pKₐ of less than about 2 include those selected from the group consisting of hydrochloric acid, hydrobromic acid, hydroiodoic acid, hydrofluoroic acid, sulfuric acid, phosphoric acid, nitric acid, trichloroacetic acid and trifluoroacetic acid, it is preferred that the strong acid of pKₐ of less than about 2 be hydrochloric acid. When the isomerization catalyst is a strong acid of pKₐ of less than about 2, it is preferred that it be used in anhydrous from or if used in as an aqueous mixture that the reaction be performed as a two phase system with the aqueous phase being separate. When the isomerization catalyst is a tertiary amine whose conjugate acid has a pKₐ greater than about 8, useful tertiary amines whose conjugate acid has a pKₐ greater than about 8 include those selected from the group consisting of (Q₃)₃N where Q₃ is C₁-C₃ alkyl, 1.8-diazabicyclo[5.4.0]undec-7-ene (DBU), 1,5-diazabicyclo[4.3.0]non-5-ene (DBN), 1,4-diazabicyclo[2.2.2]octane (DABCO), pyridine, p-dimethylaminopyridine and pyrrolidinylpyridine. When the isomerization catalyst is salt of a tertiary amine whose conjugate acid has a pKₐ greater than about 8, it is preferred that the salt of a tertiary amine whose conjugate acid has a pKₐ greater than about 8 be pyridine hydrochloride. Regardless of which chemical agent is used, only a catalytic amount is required. For example, after formation of the cis-enedione just adding commercial chloroform containing the usual impurity of hydrochloric acid is sufficient to effect the transformation to the corresponding trans-enedione. The isomerization of cis-enedione corresponding trans-enedione can be performed at 20°-25° C. (room temperature). At room temperature, the reaction usually takes a few hours. It is necessary to monitor the course of the reaction by standard methods such as LC or TLC to ensure that it does not go too long. If the reaction goes too long, the reaction reforms the 7α-substituted steroid (II) with a Δ⁶-double bond. Once the reaction has proceeded to completeness where it is desirous to terminate the reaction, the reaction can be terminated as follows. When the isomerization catalyst is an acid or salt of a tertiary amine whose conjugate acid has a pKₐ of greater than 8, one can terminate the reaction by washing with water. If aqueous acid is used as the isomerization catalyst, it is best to separate the phases and then wash the non-aqueous phase with water. If the isomerization catalyst is a tertiary amine whose conjugate acid has a pKₐ of greater than 8, then the reaction mixture is washed with aqueous aced followed by water. The trans-enedione can be isolated and purified, however it is preferred not to isolate and purify it but rather carry it on in situ.

The next step is the conversion of either the cis-enedione or trans-enedione, or mixture thereof, to the corresponding hydroperoxy compound, hydroxy compound, biscarbonyl compound and/or the carboxylic acid or mixtures thereof. The cis-enedione or trans-enedione, or mixture thereof, is transformed to the corresponding hydroxy compound, peroxy compound, or biscarbonyl compound or carboxylic by contacting the cis-enedione or trans-enedione or a mixture thereof, with ozone in the presence of an alcohol of the formula R₇-₂—OH where R₇-₂ is —H or C₁-C₄ alkyl optionally substituted with one or two —OH. This includes water, methanol, ethanol, propyl alcohol, isopropyl alcohol, ethylene glycol, glycerol, etc. It is preferred that R₇-₂ is —H, C₁ or is iso-C₃; it is more preferred that R₇-₂ is a mixture of —H, C₁ and iso-C₃. This means a mixture of water, methanol and isopropanol is the preferred R₇-₂—OH. The steroidal starting materials must be in solution using a solvent that will dissolve them at the cold temperatures at which it is preferred to perform this reaction. Methylene chloride is the preferred solvent. The reaction temperatures can be as low as about −100° up to about 40° C. It is preferred that the temperature be from about −78° to about −20° C.; it is more preferred that the temperature be about −50° C. The lower the temperature, the more selectivity; the higher the temperature the less selectivity. Hence, the actual temperature used will depend on the particular reactants used and the degree of selectivity desired. The reaction is permitted to run until the starting material is reduced to a small amount. The ozone must be stopped when the starting material is consumed or the ozone will destroy the product by reacting with the Δ⁴- and/or Δ⁹⁽¹¹⁾- double bonds if present. The alcohol, R₇-₂—OH, is used in a large excess to efficiently trap the carbonyl oxide intermediate produced. Further, the reaction temperature, the time the reaction is permitted to run and the nature of the particular alcohol, R₇-₂—OH, determines the identity of the product or if more than one product is produced, the ratio of products. If the alcohol, R₇-₂—OH, has a hindered R₇-₂ group, then the product is more likely to be the biscarbonyl compound, all other things being equal. Similarly, if the alcohol, R₇-₂—OH, does not have a hindered R₇-₂ group, such as methyl, then the product is more likely to be the hydroxy compound, all other things being equal. The preferred product produced by the oxidation process is the carboxylic acid.

The hydroperoxy compound can be converted to the corresponding hydroxy compound by contacting the hydroperoxy compound with a hydroperoxy-deoxygenating agent. It is preferred to use a mild hydroperoxy-deoxygenating agent, one which both deoxygenates-, and second does not add to the steroid molecule. Useful hydroperoxy-deoxygenating agents include those selected from the group consisting of: Q₁Q₂S where Q¹, and Q₂ are the same or different and are C₁-C₄ alkyl or phenyl, bisulfite, sulfite, thiosulfate, tetrahydrothiophene, hydrosulfite, thiourea, butyl vinyl ether, (C₁-C₄ alkyl)₃ phosphine, triphenylphosphine, and tetramethylethylene. It is preferred that the hydroperoxy-deoxygenating agent is dimethylsulfide. When the hydroperoxy-deoxygenating agent is bisulfite and sulfite, sodium and potassium are the preferred cations. One equivalent of the hydroperoxy-deoxygenating agent is required, but more than one equivalent, such as about two equivalents, are normally used to ensure that all of the hydroperoxy compound is reduced. The reaction is performed at 20-25° and is usually complete in about 1 hour. The hydroxy compound can be isolated and purified if desired, however, it is preferable to carry it on in situ without isolating or purifying it.

The hydroperoxy compound can be transformed to the corresponding carboxylic acid by contacting the hydroperoxy compound with a carboxylic acid forming agent selected from the group consisting of: (a) heat, (b) a base whose conjugate acid has a pKₐ of about 5 or above, (c) an acid which has a pKₐ of less than about 3, (d) an acylating agent. When the carboxylic acid forming agent is (a) heat, the reaction mixture should be heated to the range of from about 30° to about 120° C.; preferably from about 80° to about 90° C. When the carboxylic acid forming agent is, (b) a base whose conjugate acid has a pKₐ of about 5 or above, useful bases include inorganic bases selected from the group consisting of hydroxide, bicarbonate, and carbonate and organic bases selected from the group consisting of (Q₃)₃N where Q₃ is C₁-C₃ alkyl, DBU, DBN, DABCO, pyridine and p-dimethylaminopyridine. It is preferred that the base is bicarbonate. Sufficient base is necessary to neutralize the steroid acid produced and any additional acid by-products. When the carboxylic acid forming agent is, (c) an acid which has a pKₐ of less than about 3, useful acids include those selected from the group consisting of hydrochloric acid, sulfuric acid, phosphoric acid, nitric acid and organic acids of the formula of R_{acid}-₁—COOH where R_{acid}-₁ is —H and C₁-C₃ alkyl optionally substituted with 1 thru 3 —Cl and —F; preferred are formic acid and trifluoroacetic acid. While catalytic amounts of acid are sufficient, several equivalent are preferred. When the carboxylic acid forming agent is, (d) an acylating agent, useful acylating agents are selected from the group consisting of R_{acid}-₂—CO—O—CO—R_{acid}-₂ is —H, C₁-C₃ alkyl optionally substituted with 1 thru 3 —Cl and —F and -phenyl. It is preferred that acylating agent is acetic anhydride or trifluoroacetic anhydride. One equivalent of the acylating agent is required. When using an acylating agent, it is preferred to use it with an acylation catalyst. Preferred acylation catalysts are pyridine and p-dimethylaminopyridine (DMAP). With regard to solvents, it is important to perform the process under homogenous reaction conditions to avoid decomposition of the hydroperoxy compound. This means using one phase conditions. Therefore, the solvent of choice will depend on the carboxylic acid forming agent used. If the carboxylic acid forming agent requires water to dissolve the reagent such as when the carboxylic acid forming agent is bicarbonate, then a water miscible organic solvent such as acetone, methanol, DMF or isopropanol is required. If the carboxylic acid forming agent is pyridine then the organic solvent can be a water immiscible organic solvent such as acetonitrile, methylene chloride or ethyl acetate. Hence, the selection of the solvent depends on the nature of the carboxylic acid forming agent used as is known to those skilled in the art. With the exception of the carboxylic acid forming agent (a) heat, the other acid forming agents (b), (c) and (d) can all be reacted at 20°-25° C. The reaction is quite fast and is usually over in less than one hour.

Both the hydroxy compound and the biscarbonyl compound are converted to the corresponding carboxylic acid in the same manner. The process involves contacting the hydroxy compound or the biscarbonyl compound, or mixture thereof, with an oxidatively cleaving agent. Useful oxidatively cleaving agents are selected from the group consisting of: (1) hydrogen peroxide with a carboxylic acid forming agent selected from the group consisting of: (a) heat, (b) a base whose conjugate acid has a pKₐ of about 5 or above, (c) an acid which has a pKₐ of less than about 3, (d) an acylating agent and an acylation catalyst; (2) KSSO₅; (3) hydrogen peroxide with a ketone selected from the group consisting of Q₄-CO-Q₅ where Q₄ and Q₅ are the same or different and are: C₁-C₄ alkyl optionally substituted with 1 thru 9 —Cl or —F, Where the Q₄ and Q₅ are taken together with the attached carbon atom to form a cyclic ketone of 5 thru 7 members, and ketones of the formula:

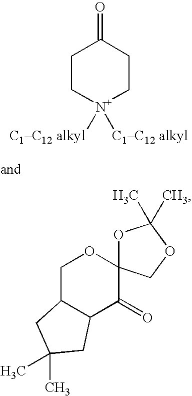

(4) hydrogen peroxide in combination with methyltrioxorhenium, (5) phenyl-C(CH₃)₂—O—OH or an alkylhydroperoxide in combination with a metal containing activator, where alkyl is from C₄-C₁₀ alkyl and metal containing activator is selected from the group consisting of Ti(isopropoxide)₄, peroxotungstophosphate, VO(acetylacetonate)₂ and Mo hexacarbonyl; (6) peracids selected from the group consisting of (a) perbenzoic acid optionally substituted with 1 or 2 —Cl or —NO₂, (b) percarboxylic acids of the formula Cₙ₂(Q₆)2ₙ₂₊₁—CO₃H where n₂ is 1 thru 4 and Q₆ is —H, —Cl or —F, (c) perphthalic acid, (d) magnesium peroxyphthalate. It is preferred that the oxidatively cleaving agent is hydrogen peroxide with a carboxylic acid forming agent. When the carboxylic acid forming agent are (a) heat, (b) a base whose conjugate acid has a pKₐ of about 5 or above, (c) an acid which has a pKₐ of less than about 3 or (d) an acylating agent and an acylation catalyst, they should be used in the same manner as discussed above for the transformation of the hydroperoxy compound to the corresponding carboxylic acid. As stated above, one equivalent of the oxidatively cleaving agent is required. Two equivalents are normally used and the reaction is monitored so that when the reaction nears completion it is stopped, or quenched, and worked up before the oxidatively cleaving agent attacks the Δ⁴- and/or Δ⁹⁽¹¹⁾-steroid double bonds. Hydrogen peroxide and bicarbonate are preferred as the oxidatively cleaving agent. With regard to solvents it is important to perform the process under homogenous reaction conditions, meaning one phase conditions. Therefore, the solvent of choice will depend on the oxidatively cleaving agent used. If the carboxylic acid forming agent requires water to dissolve the reagent such as when the carboxylic acid forming agent is bicarbonate, then a water miscible organic solvent such as acetone, DMF, methanol or isopropanol is required. If the carboxylic acid forming agent is pyridine then the organic solvent can be a water immiscible organic solvent such as acetonitrile, methylene chloride or ethyl acetate. Hence, the selection of the solvent depends on the nature of the carboxylic acid forming agent used as is known to those skilled in the art. With the exception of the carboxylic acid forming agent (a) heat, the other acid forming agents (b), (c) and (d) can all be reacted at 20-25° C. The reaction is quite fast and is usually over in less than one hour. If the reaction mixture contains some hydroperoxy compound, then it is useful to first treat the reaction mixture with a hydroperoxy-deoxygenating agent. It is preferred that the hydroperoxy-deoxygenating agent is dimethylsulfide.

In one preferred embodiment of the sixth step of Scheme I, the compound of Formula XXIX is a compound of Formula XXIXA, as shown above, and the compound of Formula XXX is a compound of Formula XXXA: 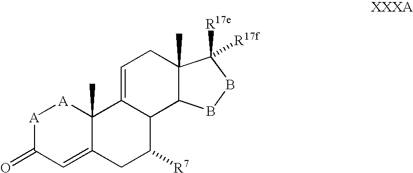
wherein
-A-A- represents the group —CH₂—CH₂— or —CH═CH—;
—B—B— represents the group —CH₂—CH₂— or an α- or β-oriented group: 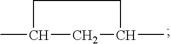
R⁷ is selected from the group consisting of hydrogen, hydroxycarbonyl, and alkoxycarbonyl; and
R^{17e} is hydroxy and R^{17f} is hydroxycarbonylalkyl, or R^{17e} and R^{17f} together with the carbon to which they are attached form a lactone ring.

In a particularly preferred embodiment of the sixth step of Scheme I, the compound of Formula XXIX is: 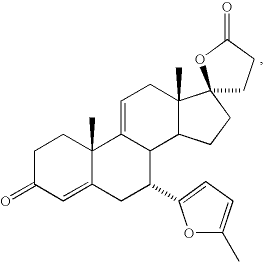
and the compound of Formula XXX is:

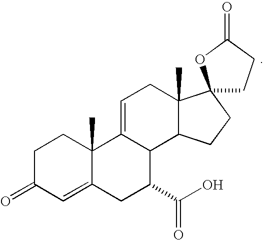

In the seventh step of Scheme I, the compound of Formula XXX is alkylated as described below to form a compound of Formula XXXI: 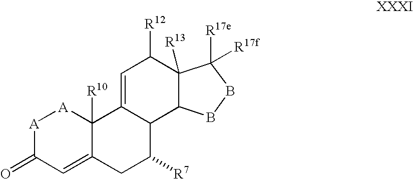
wherein R³, R¹⁰, R¹², R¹³, -A-A-, and —B—B— are as defined above for Formula XXI; R^{17e} and R^{17f} are as defined above for Formula XXIII; and R₇ is alkoxycarbonyl.

The reaction to form the 7-alkoxycarbonyl steroid of Formula XXXI proceeds via a 5,7-lactone intermediate of Formula XXX-1: 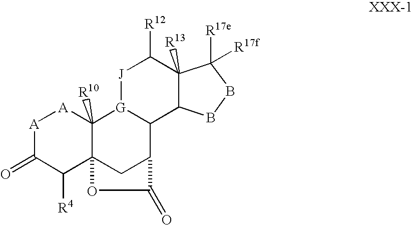
wherein R¹⁰, R¹², R¹³, -A-A-, and —B—B— are as defined above for Formula XXI; R⁴, R^{17e}, and R^{17f} are as defined above for Formula XXIII; and -D-D-, -G-J-, and -E-E- are as defined above for Formula XXVIII.

The 5,7-lactone intermediate of Formula XXX-1 may be formed by contacting a compound of Formula XXX with a reaction medium which has a pH of less than about 5. The conversion of the 7-carboxylic acid to the corresponding 5,7-lactone intermediate is an equilibrium reaction. The lower the pH used for the reaction medium the more the equilibrium shifts towad the 5,7-lactone, hence the desire to keep the pH less than 5 and preferably in the range of 1 through 5. It is preferred to perform the reaction under anhydrous conditions; under anhydrous conditions it is preferred that the acid be a strong acid of pKₐ less than about 2. Useful strong acids include those selected from the group consisting of fluorosulfonic, chlorosulfonic, benzenesulfonic, p-toluenesulfonic, methanesulfonic, trifluoromethanesulfonic, trifluoracetic, trichloroacetic, hydrochloric, sulfuric, phosphoric, and nitric; it is preferred that the acid is benzenesulfonic, p-toluenesulfonic or methanesulfonic acid. Alternatively, the process can be performed using aqueous acid as the catalyst. Under these conditions it is preferred to perform the process in a two-phase system. The amound of acid used is not very important and can be present in an amount from catalytic to excess. Bases are also operable to catalyze the reation of the carboxylic acid to the corresponding 5,7-lactone as long as they are used in a catalytic amount. Useful bases include those selected from the group consisting of hydroxide, bicarbonate, carbonate, DBU, DBN, DABCO, pyridine, P-dimethylaminopyridine, Q₇-COO⁻ (where Q₇ is hydrogen, C₁-C₃ alkyl, or phenyl), and (Q₃)₃N (where Q₃ is C₁-C₃ alkyl); preferred are hydroxide, bicarbonate, carbonate, triethylamine or pyridine. The solvents for the transformation of the carboxylic acid to the corresponding 5,7-lactone are helpful in effecting the equilibrium of the reaction. It is preferred to use a solvent in which the starting carboxylic acid is soluble and in which the 5,7-lactone is not soluble. That way the 5,7-lactone precipitates out as it is formed pushing the equilibrium towards the desired 5,7-lactone. A preferred solvent is acetone. This reaction is performed afrom about 0° to about 25° C. and is complete in a few hours. Depending on the pH of the reaction medium and solvent used, ratios of <95/5 of carboxylic acid/5,7-lactone are obtained. Since this process step is an equilibrium reaction, the pH of the reaction medium helps control the final position of the equilibrium as is known to those skilled in the art.

Alternatively, the 5,7-lactone intermediate of Formula XXX-1 may be formed by contacting the carboxylic acid of Formula XXX under anhydrous conditions with an anhydrous reaction medium of pH less than about 5. It is preferred that the reaction medium contains an acid which has a pKa of less than about 4. Useful acids which have a pKa of less than about 4 include those selected from the group consisting of fluorosulfonic, chlorosulfonic, benzenesulfonic, p-toluenesulfonic, methanesulfonic, trifluoromethanesulfonic, trifluoroacetic, trichloroacetic, hydrochloric, sulfuric, phosphoric, and nitric. It is preferred that the acid is benzenesulfonic, p-toluenesulfonic or methanesulfonic. It is also preferred that the carboxylic acid is reacted with the acid in a two-phase system. The process also includes reacting the carboxylic acid with a catalytic amount of base. Useful bases include those selected from the group consisting of hydroxide, bicarbonate, carbonate, DBU. DBN, DABCO, pyridine, p-dimethylaminopyridine, Q₇-COO⁻ (where Q₇ is hydrogen, C₁-C₃ alkyl, or phenyl), and (Q₃)₃N (where Q₃ is C₁-C₃ alkyl).

The 5,7-lactone intermediate of Formula XXX-1 is then converted to the 7α-alkoxycarbonyl of Formula XXXI by contacting the 5,7-lactone with base to form a reaction mixture, and contacting this reaction mixture with an alkylating agent. The base needs to be strong enough to open the 5,7-lactone but of the type that will not react with the alkylating agent, a weak nucleophile. Useful bases include those selected from the group consisting of bicarbonate, carbonate, hydroxide, and C₁-C₄ alkoxide. It is preferred that the base is bicarbonate. The amount of base required is from about 1 to about 1.5 equivalents. Useful alkylating agents include those selected from the group consisting of dimethylsulfate, methyl iodide, methyl bromide, trimethylphosphate, dimethylcarbonate, and methyl chloroformate; preferrred is dimethylsulfate. The amount of alkylating agent used should be the same as the number of equivalents of base used or a very slight excess over that. The preferred method of the process is to react it in a sequential manner in a two-step reaction with base first and then the alkylating agent. If the reaction is performed all in one step, the base may react with the alkylating agent necessitating more base and more alkylating agent. The more efficient way is to first react the 5,7-lactone with at least one equivalent of base, preferably from about 1 to about 1.5 equivalents, and then to react the salt of the carboxylic acid which is formed with the alkylating agent. The solvent used will depend on the nature of the base used. If it is water soluble, such as bicarbonate or hydroxide, then a mixture of water and a water miscible organic solvent is preferred. These water miscible organic solvents include methanol, ethanol, isopropanol, acetone, THF and DMF. If the base is water soluble and the solvent is a mixture of water and a water immiscible solvent, then a phase transfer catalyst, such as tetrabutylammonium bisulfate or tributylmethylammonium chloride is used. If the base is soluble in a water immiscible organic solvent, one that will also dissolve the 5,7-lactone, then a water-immiscible organic solvent is suitable. The reaction temperature is dependent on the reactiviy of the alkylating agent. If an agent such as dialkycarbonate is used the reaction will go slowly and heating up to about 150° C. may be necessary. On the other hand, if a more reactive agent such as dialkylsulfate is used the reaction goes in about 1 hour at 40° C. While in theory one equivalent of base and one equivalent of alkylating agent should be sufficient, in practice more than one equivalent is needed for the optimum reaction conditions.

In one preferred embodiment of the seventh step of Scheme I, the compound of Formula XXX is a compound of Formula XXXA, as shown above, and the compound of Formula XXXI is a compound of Formula XXXIA: 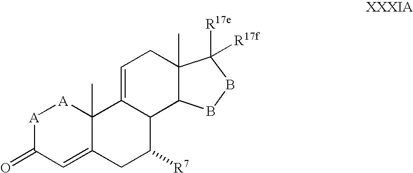
wherein
-A-A- represents the group —CH₂—CH₂— or —CH═CH—;
R³ is lower alkoxy;
—B—B— represents the group —CH₂—CH₂— or an α- or β-oriented group: 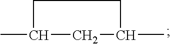
   and
R^{17e} is hydroxy and R^{17f} is hydroxycarbonylalkyl, or R^{17e} and R^{17f} together with the carbon to which they are attached form a lactone ring.

In a particularly preferred embodiment of the seventh step of Scheme I, the compound of Formula XXX is: 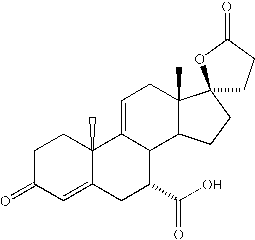
and the compound of Formula XXXI is:

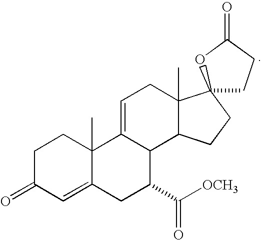

In the eighth step of Scheme I, the compound of Formula XXXI is epoxidized by means well known to those skilled in the art to form a compound of Formula XXXII as shown above.

In one preferred embodiment of the eighth step of Scheme I, the compound of Formula XXXI is a compound of Formula XXXIA, as shown above, and the compound of Formula XXXII is a compound of Formula XXXIIA: 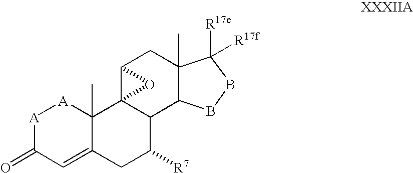
wherein
-A-A- represents the group —CH₂—CH₂— or —CH═CH—;
R³ is lower alkoxy;
—B—B— represents the group —CH₂—CH₂— or an α- or β-oriented group: 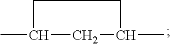
   and
R^{17e} is hydroxy and R^{17f} is hydroxycarbonylalkyl, or R^{17e} and R^{17f} together with the carbon to which they are attached form a lactone ring.

In a particularly preferred embodiment of the eighth step of Scheme I, the compound of Formula XXXI is: 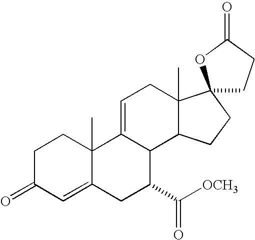
and the compound of Formula XXXII is eplerenone:

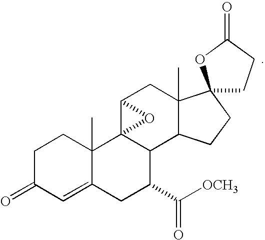

Methods for 6,7-dehydrogenation of a 9(11)-unsaturated 3-methyl enol ether steroid are described hereinbelow. Methods for converting steroid compounds of Formula XXIX to Eplerenone are more fully described in co-assigned U.S. patent application Ser. No. 10/392,833 (entitled “Processes to Prepare Eplerenone”, filed 21 Mar. 2003 and incorporated by reference herein in its entirety) and the equivalent co-assigned PCT Publication No. 03/082895 (entitled “Process to Prepare Eplerenone” and incorporated by reference herein in its entirety; in particular, see Chart A and the accompanying text at page 50, line 13 through page 85, line 23). Further, methods for the oxidation of the enol ether substrate are more fully described in co-assigned U.S. patent application Ser. No. 10/392,857 (entitled “C-17 Spirolactonization and 6,7 Oxidation of Steroids,” filed on even date herewith and incorporated by reference herein in its entirety).

Generally, the epoxidation process of the invention is conducted in accordance with the procedure describe in U.S. Pat. No. 4,559,332, as more particularly described in U.S. Pat. No. 5,981,744, col. 40, line 38 to col. 45, line 15 and in Examples 26-28 and 42-51. See also U.S. Pat. No. 6,610,844. The U.S. Pat. Nos. 4,559,332, 5,981,744 and 6,610,844 patent documents are expressly incorporated herein by reference.

In the epoxidation process as described in these references, a solution of Δ^{9,11} substrate in a suitable solvent is contacted with an aqueous hydrogen peroxide composition in the presence of an activator such as, for example, trichloracetonitrile or, preferably, trichloroacetamide. With the goal of assuring complete conversion of the substrate to the 9,11-epoxide, the epoxidation reaction as described in the above-cited references is typically conducted at a molar charge ratio of ≧10 moles hydrogen peroxide per mole steroid substrate.

It has now been discovered that the epoxidation reaction can be conducted at a significantly lower ratio of hydrogen peroxide to Δ^{9,11} substrate than is taught or exemplified in U.S. Pat. Nos. 4,559,332, 5,981,744 or U.S. Pat. No. 6,610,844. Operation at a relatively low peroxide to substrate ratio provides the option of achieving any of several potential advantages, as discussed hereinbelow.

In carrying out the reaction, preferably the solution of substrate, together with the activator and a buffer are first charged to a reaction vessel comprising an epoxidation reaction zone, and an aqueous solution of hydrogen peroxide added thereto. Preferably, a solvent for the steroid substrate is selected in which the solubility of the steroid substrate and epoxidized steroid product is reasonably high, preferably at least about 10 wt. %, more preferably at least about 20 wt. %, but in which the solubility of water is low, preferably less than about 1 wt. %, more preferably less than about 0.5 wt. %. In such embodiments, an epoxidation reaction zone comprising a two phase liquid reaction medium that is established within the reaction vessel, with the substrate in the organic phase and hydrogen peroxide in the aqueous phase. Epoxidation of the substrate in the two phase medium produces a reaction mass containing the epoxidized steroid reaction product substantially within the solvent phase. Without being held to a particular theory, it is believed that the reaction occurs in the organic phase or at the interface between the phases, and that more than a very minor water content in the organic phase effectively retards the reaction.

After the solution of steroid is introduced into the reactor, the entire peroxide solution may be added over a short period of time before reaction is commenced, e.g., within 2 to 30 minutes, more typically 5 to 20 minutes. Where the strength of the peroxide solution as supplied to the reactor is greater than the concentration to be established at the outset of the reaction, water may be charged and mixed with the organic phase prior to addition of peroxide, water being added in a volume which thereafter dilutes the peroxide concentration to the level desired at the outset of the reaction. In those embodiments wherein hydrogen peroxide is introduced at the beginning of the reaction cycle, the solvent phase and added aqueous peroxide solution are preferably maintained at a relatively low temperature, more preferably, lower than about 25° C., typically lower than about 20° C., more typically in the range of about −5° to about 15° C., as the peroxide is introduced.

Reaction then proceeds under agitation. Preferably the reaction is conducted under an inert atmosphere, preferably by means of a nitrogen purge of the reactor head space.

Generically, the peroxide activator may correspond to the formula:
R^{o}C(O)NH₂
where R^{o} is a group having an electron withdrawing strength (as measured by sigma constant) at least as high as that of the monochloromethyl group. Preferably, the promoter comprises trichloroacetonitrile, trichloracetamide, or a related related compound corresponding to the formula: 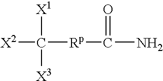
where X¹, X², and X³ are independently selected from among halo, hydrogen, alkyl, haloalkyl and cyano and cyanoalkyl, and R^{p} is selected from among arylene and —(CX⁴X⁵)ₙ—, where n is 0 or 1, at least one of X¹, X², X³, X⁴ and X⁵ being halo or perhaloalkyl. Where any of X¹, X², X³, X⁴ or X⁵ is not halo, it is preferably haloalkyl, most preferably perhaloalkyl. Particularly preferred activators include those in which n is 0 and at least two of X¹, X² and X³ are halo; or in which all of X¹, X², X³, X⁴ and X⁵ are halo or perhaloalkyl. Each of X¹, X² X³, X⁴ and X⁵ is preferably Cl or F, most preferably Cl, though mixed halides may also be suitable, as may perchloralkyl or perbromoalkyl and combinations thereof.

Other suitable promoters include hexafluoroacetone dicyclohexylcarbodiimide.

The buffer stablizes the pH of the reaction mass. Without being bound to a particular theory, the buffer is further believed to function as a proton transfer agent for combining the peroxide anion and promoter in a form which reacts with the Δ^{9,11} substrate to form the 9,11-epoxide. It is generally desirable that the reaction be conducted at a pH in the range of about 5 to about 8, preferably about 6 to about 7. Suitable compounds which may function both as a buffer and as a proton transfer agent include dialkali metal phosphates, and alkali metal salts of dibasic organic acids, such as Na citrate or K tartrate.

Especially favorable results are obtained with a buffer comprising dipotassium hydrogen phosphate, and/or with a buffer comprising a combination of dipotassium hydrogenphosphate and potassium dihydrogen phosphate in relative proportions of between about 1:4 and about 2:1, most preferably in the range of about 2:3. Borate buffers can also be used, but generally give slower conversions than dipotassium phosphate or KH₂PO₄ or K₂HPO₄/KH₂PO₄ mixtures. Whatever the makeup of the buffer, it should provide a pH in the range indicated above. Aside from the overall composition of the buffer or the precise pH it may impart, it has been observed that the reaction proceeds much more effectively if at least a portion of the buffer is comprised of dibasic hydrogenphosphate ion. It is believed that this ion may participate essentially as a homogeneous catalyst in the formation of an adduct or complex comprising the promoter and hydroperoxide ion, the generation of which may in turn be essential to the overall epoxidation reaction mechanism. Thus, the quantitative requirement for dibasic hydrogenphosphate (preferably from K₂HPO₄) may be only a small catalytic concentration. Generally, it is preferred that a dibasic hydrogenphosphate be present in a proportion of at least about 0.1 equivalents, e.g., between about 0.1 and about 0.3 equivalents, per equivalent substrate.

After addition of the peroxide solution is substantially complete, the temperature may be raised, e.g., into the range of 15° to 50° C., more typically 20° to 40° C. to enhance the rate of the reaction and the conversion of substrate to epoxide. Optionally, the peroxide solution can be added progressively over the course of the reaction, in which case the temperature of the reaction mass is preferably maintained in a range of about 15° to about 50° C., more preferably between about 20° and about 40° C. as the reaction progresses. In either case, the reaction rate in the two phase reaction medium is ordinarily mass transfer limited, requiring modest to vigorous agitation to maintain a satisfactory reaction rate. In a batch reactor, completion of the reaction may require from 3 to 24 hours, depending on the temperature and intensity of agitation.

The decomposition of hydrogen peroxide is an exothermic reaction. At ordinary reaction temperatures the rate of decomposition is small to negligible, and the heat generated is readily removed by cooling the reaction mass under temperature control. However, if the reaction cooling system or temperature control system fails, e.g., by loss of agitation, the rate of decomposition can be accelerated by the resulting increase in temperature of the reaction mass, which can in turn accelerate the rate of autogenous reaction heating. Where the initial molar ratio of peroxide to steroid substrate is in the range described in U.S. Pat. No. 4,559,332, U.S. Pat. No. 5,981,744 or U.S. Pat. No. 6,610,844, i.e., in the range of 10:1 or higher, autogenous heating as resulting from loss of cooling can reach a temperature at which the decomposition becomes autocatalytic, and thus very rapid and uncontrolled, resulting in potential eruption of the reaction mass. If the temperature is high enough, destructive oxidation of the steroid substrate may generate additional reaction heat, further accelerating the rate of temperature increase and the severity of the resulting eruption. Events other than loss of agitation can also potentially destabilize the peroxide and result in an exotherm that leads to uncontrolled decomposition. For example, contaminants such as rust or other source of transition metals in the peroxide or substrate solutions may catalyze a rapid or uncontrolled release of oxygen from the aqueous phase.

It has now been discovered that the epoxidation reaction can be conducted at a significantly lower ratio of peroxide to Δ^{9,11} substrate than is taught or exemplified in U.S. Pat. Nos. 4,559,332, 5,981,744 or U.S. Pat. No. 6,610,844, thereby reducing the risk of uncontrolled decomposition of the peroxide. More particularly, it has been discovered that the reaction can be conducted at a charge ratio between about 2 and about 7 moles, preferably between about 2 and about 6 moles, more preferably between about 3 and about 5 moles hydrogen peroxide per mole Δ^{9,11} substrate. Operation at such relatively low ratios of peroxide to substrate reduces the extent to which the reaction mass may be heated by autogenous decomposition of the peroxide. Preferably, the peroxide to substrate ratio is low enough so that the maximum temperature attainable by autogenous heating is lower than the threshold temperature for autocatalytic decomposition, which may entirely preclude decomposition of the peroxide from reaching the stage at which an eruption of the reaction mass could result. Operation at the above described charge ratios makes this feasible.

Further protection against uncontrolled reaction is provided where the epoxidation reaction is conducted at a relatively modest temperature below the temperature of incipient decomposition of the peroxide, or where the rate of decomposition is relatively slow. Thus, in the event of a process upset which results in accumulation of unreacted hydrogen peroxide, little autogenous heating can occur, at least initially, so that, even after loss of agitation, reactor cooling capacity remains sufficient under natural circulation to maintain the temperature of the reaction mass in a safe range, or at least process operators are afforded ample time to take corrective measures before conditions for an uncontrolled autocatalytic decomposition are approached. For this purpose, it is preferred that the epoxidation reaction be carried out at a temperature in the range of about 0° to 50° C., more preferably in the range of about 20° to about 40° C.

Still further protection against uncontrolled reaction is afforded by conducting the epoxidation reaction in a liquid reaction medium comprising a solvent having a boiling point at the reaction pressure that is well below the autocatalytic decomposition temperature of the peroxide, and preferably only modestly higher than the reaction temperature. Preferably, the boiling point of the organic phase of the reaction mixture is no greater than about 60° C., preferably not greater than about 50° C. Preferably, the selected solvent does not boil from the reaction mass at the reaction temperature, but is rapidly vaporized if the temperature increases by a modest increment from about 10 centigrade degrees to about 50 centigrade degrees, whereby the heat of vaporization serves as a heat sink precluding substantial heating of the reaction mass until the solvent shall have been substantially driven out of the reaction zone. Where the reaction is conducted under atmospheric pressure at a temperature in the aforesaid ranges, a variety of solvents are available which meet these criteria, and are also suitable for the epoxidation reaction. These include methylene chloride (atmos. b.p.=39.75° C.), dichloroethane (atmospheric b.p.=83° C., and methyl t-butyl ether (b.p.=55° C.).

The water content of the reaction mass also serves as a substantial sensible heat sink. Where the reaction is conducted at, near or below atmospheric pressure, the water content of the aqueous hydrogen peroxide solution serves as a potentially much larger heat sink, though it is generally preferred to avoid conditions under which substantial steam generation occurs since this may also result in eruption of the reaction mass, albeit much less violent than that which results from autocatalytic decomposition of a peroxide compound.

Thus, in one aspect, the present invention comprises conducting the epoxidation reaction in a liquid reaction medium, preferably comprising a solvent for the steroid, which contains the steroid substrate and peroxide in such absolute and relative proportions, and at a relatively modest initial epoxidation reaction temperature, such that the decomposition of the peroxide content of the reaction mass in stoichiometric excess vs. the substrate charge does not, and preferably cannot, produce an exotherm effective to initiate autocatalytic decomposition of hydrogen peroxide, or at least not to cause autocatalytic decomposition to proceed an an uncontrolled rate. To protect against an uncontrolled decomposition at any time during the epoxidation cycle, it is further preferred that the aforesaid combination of conditions be such that decomposition of the entire peroxide content of the reaction mass, at any time during the course of the reaction, cannot produce an exotherm effective to initiate autocatalytic decomposition of hydrogen peroxide, or at least not to cause autocatalytic decomposition thereof to proceed at an uncontrolled rate. Optimally, the combination of substrate concentration, peroxide compound concentration and initial temperature are such that decomposition oif the stoichiometeric excess, or of the entire peroxide compound charge, cannot produce an exotherm sufficient to initiate autocatalytic decomposition, or at leat not to cause an uncontrolled autocatalytic decomposition, even under adiabatic conditions, i.e., upon loss of cooling in a well-insulated reactor.

The peroxide content of the aqueous phase, as established at the outset of the epoxidation reaction, is preferably between about 25% and about 50% by weight, more preferably between about 25% and about 35% by weight, and the initial concentration of Δ^{9,11} steroid substrate in the organic phase is between about 3% and about 25% by weight, more preferably between about 7% and about 15% by weight. Preferably, components effective to promote the epoxidation reaction such as, for example, trichloroacetonitrile or trichloroacetamide, together with a phosphate salt such as a dialkali hydrogen phosphate, are charged to the reactor with the steroid solution, prior to addition of the aqueous peroxide. The molar ratio of peroxide to phosphate is preferably maintained in the range between about 10:1 and about 100:1, more preferably between about 20:1 and about 40:1. The initial trichloroacetamide or trichloroacetonitrile concentration is preferably maintained at between about 2 and about 5 wt. %, more preferably between about 3 and about 4 wt. %, in the organic phase; or in a molar ratio to the steroid substrate between about 1.1 and about 2.5, more preferably between about 1.2 and about 1.6. The volumetric ratio of the aqueous phase to the organic phase ultimately introduced into the reactor is preferably between about 10:1 and about 0.5:1, more preferably between about 7:1 and about 4:1. As mentioned above, and again without being held to a particular theory, it is believed that the epoxidation reaction occurs in the organic phase or at the interface between the phases. In any event, the reaction mass is preferably agitated vigorously to promote transfer of peroxide to the organic phase, or at least to the interface. A high rate of mass transfer is desired both to promote the progress of the reaction, thereby shortening batch reaction cycles and enhancing productivity, and to minimize the inventory of peroxide in the reaction vessel at any given rate of addition of aqueous peroxide solution to the reaction mass. Thus, in various preferred embodiments of the invention, the agitation intensity is at least about 10 hp/1000 gal. (about 2 watts/liter, typically from about 15 to about 25 hp/1000 gal. (about 3 to about 5 watts/liter). The epoxidation reactor is also provided with cooling coils, a cooling jacket, or an external heat exchanger through which the reaction mass is circulated for removal of the heat of the epoxidation reaction, plus any further increment of heat resulting from decomposition of the peroxide.

After completion of the epoxidation reaction, unreacted hydrogen peroxide in the aqueous phase is preferably decomposed under controlled conditions under which release of molecular oxygen is minimized or entirely avoided. A reducing agent such as an alkali metal sulfite or alkali metal thiosulfate is effective for promoting the decomposition. Preferably, the aqueous phase of the final reaction mass, which comprises unreacted peroxide, is separated from the organic phase, which comprises a solution of 9,11-epoxidized steroid product in the reaction solvent. The aqueous phase may then be “quenched” by contact of the peroxide contained therein with the reducing agent.

Where the molar charge ratio of peroxide to steroid substrate is in the range of, for example, 3 to 5, and the initial concentration of a peroxide in the aqueous phase is in the range of about 7 to about 9 molar concentration (i.e., 25% to 30% by weight in the case of hydrogen peroxide), the spent aqueous peroxide solution at the end of the reaction contains about 4-6 molar concentration % peroxide (between about 15 and about 21% by weight for hydrogen peroxide). Prior to phase separation, the aqueous phase may be diluted with water to reduce the peroxide concentration and thereby the likelihood and extent of any exotherm resulting from decomposition during the phase separation and/or transfer of the aqueous phase, such as transfer to another vessel for quenching with a reducing agent. For example, sufficient water may be added to reduce the concentration of hydrogen peroxide in the spent aqueous phase to between about 2% and about 10% by weight, more preferably between about 2% and about 5% by weight.

Quenching may be effected by adding the spent aqueous peroxide solution, or a dilution thereof, to a vessel containing an aqueous solution of the reducing agent, or vice-versa. According to one alternative, the organic phase may be transferred to a separate vessel upon separation from the aqueous phase, and the aqueous phase allowed to remain in the reaction vessel. The solution of the reducing agent may then be added to the diluted or undiluted aqueous phase in the reaction vessel to effect reduction of the residual peroxide. Alternatively, the diluted or undiluted peroxide solution may be added over time to a vessel to which an appropriate volume of reducing agent solution has initially been charged. Where the reducing agent is an alkali metal sulfite, the sulfite ion reacts with the peroxide to form sulfate ion and water.

The decomposition reaction is highly exothermic. Decomposition is preferably conducted at a temperature controlled in the range of between about 20° C. and about 50° C. by transfer of heat from the aqueous mass in which the decomposition proceeds. For this purpose, the quenching reactor may be provided with cooling coils, a cooling jacket, or an external heat exchanger through which the quench reaction mass may be circulated, for transfer of decomposition reaction heat to a cooling fluid. The quenching mass is preferably subjected to moderate agitation to maintain uniform distribution of reducing agent, uniform temperature distribution, and rapid heat transfer.

Where the reducing agent is added to the spent peroxide solution, addition is preferably carried out at a rate controlled to maintain the temperature of the quench reaction mass in the aforesaid range, thereby to effect controlled decomposition of the peroxide.

The alternative process, i.e., the process wherein the peroxide solution is added to the reducing agent solution, avoids the presence of a large inventory of peroxide that might otherwise be subject to autocatalytic decomposition as triggered by the addition of a decomposition agent thereto. However, this alternative requires transfer of the spent peroxide solution while the reverse alternative allows the peroxide solution to be retained in the epoxidation reactor while only the organic phase of the reaction mass and the reducing agent solution need to be transferred. Regardless of which alternative is followed, the quench reaction is preferably conducted in the temperature range specified above.

For purposes of the quenching reaction, the aqueous quench solution charged to the quenching reaction zone preferably contains between about 12 wt % and about 24 wt. %, more preferably between about 15 wt % and about 20 wt. %, of a reducing agent such as Na sulfite, Na bisulfite, etc. The volume of quench solution is preferably sufficient so that the reducing agent contained therein is in stoichiometric excess with respect to the peroxide content of the aqueous phase to be quenched. The volumetric ratio of quench solution that is mixed with the peroxide solution may typically vary from about 1.2 to about 2.8, more typically from about 1.4 to about 1.9 after preliminary water dilution of the spent aqueous peroxide solution.

Typically, residual organic solvent may have remained in the reactor after the initial phase separation, and have become entrained in the aqueous phase during the quenching reaction. Also, the quenched aqueous phase may contain a salt of trichloroacetic acid, formed as a by-product of the epoxidation reaction when trichloroacetamide is used as a promoter. Before disposal of the quenched aqueous phase, entrained reaction solvent is preferably removed therefrom, e.g., by solvent stripping. If a solvent such as methylene chloride is entrained in the quench reaction mixture, and the aqueous phase thereof contains trichloroacetate, the aqueous phase is preferably heated prior to solvent stripping in order to decarboxylate the trichloroacetate. Decarboxylation of the trichloroacetate may be achieved by heating to a temperature of, e.g., 70° C. or higher. If trichloroacetate is not removed, it can decompose during solvent stripping to produce chloroform and carbon dioxide.

After separation from the aqueous phase of the reaction mass, the organic phase is preferably washed with water to remove unreacted peroxide and any inorganic contaminants. For elimination of residual peroxide it may be useful for the wash water to contain a reducing agent. For example, the organic phase may be contacted with an aqueous wash solution having a pH in the range of 4 to 10 and containing typically 0.1 to 5 mole % reducing agent, preferably about 0.2 to about 0.6 mole % reducing agent (such as, e.g., 6 to 18% aqueous solution of Na sulfite), in a convenient volumetric ratio of wash solution to organic phase between about 0.05:1 to about 0.3:1. After separation of the spent reducing agent wash from the organic phase, the organic phase is preferably washed sequentially with a dilute caustic solution (e.g., 0.2% to 6% by weight NaOH in a volumetric ratio to the organic phase between about 0.1 to about 0.3) followed by either a water wash or a dilute acid solution (for example, a 0.5 to 2 wt. % HCl solution in a volumetric ratio to the organic phase between about 0.1 and about 0.4). A final wash with further Na bisulfite or Na metabisulfite or Na sulfite solution may also be conducted.

If a solvent such as methylene chloride is entrained in the dilute caustic wash, the aqueous phase thereof contains trichlorosodiumacetate produced from basic hydrolysis of residual trichloroacetamide, the aqueous phase is preferably heated prior to solvent stripping in order to decarboxylate the trichlorosodiumacetate. Decarboxylation of the trichlorosodiumacetate may be achieved by heating to a temperature of, e.g., 70° C. or higher. The caustic wash may be combined with the quenched aqueous phase of the reaction mixture for purposes of decarboxylation and residual solvent stripping.

The washed organic phase is concentrated by evaporation of solvent, for example, by atmospheric distillation, resulting in precipitation of steroid to form a relatively thick slurry with about 40% to about 75% by weight contained steroid. Where mother liquor from a recrystallization step is recycled, as described below, the mother liquor may be mixed with the steroid slurry, and the solvent component of the mother liquor removed by vacuum to again produce a thick slurry having a solids concentration typically in the same range as the slurry obtained by removing the reaction solvent. A solvent in which the solubility of the steroid product is relatively low, e.g., a polar solvent such as ethanol, is added to the slurry obtained from removal of reaction solvent, or to the second slurry as obtained by removal of the recrystallization mother liquor solvent. Alternative solvents include toluene, acetone, acetonitrile and acetonitrile/water. In this step, the impurities are digested into the solvent phase, thus refining the solid phase steroid product to increase its assay. Where the digestion solvent is an alcohol such as ethanol, it may be added in a volumetric ratio of ethanol to contained steroid between 6 and about 20. A portion of the ethanol and residual organic solvent are removed from from the resulting mixture by distillation, yielding a slurry typically containing between about 10 wt. % and about 20 wt. % steroid product, wherein impurities and by-products are substantially retained in the solvent phase. Where the solvent is ethanol, the distillation is preferably conducted at atmospheric presusre or slightly above.

After distillation of the digestion solvent, the steroid product solids are separated from the residual slurry, e.g., by filtration. The solid product is preferably washed with the digestion solvent, and may be dried to yield a solid product substantially comprising the 9,11-epoxy steroid. Drying may advantageously be conducted with pressure or vacuum using an inert carrier gas at a temperature in the range of about 35 to about 90° C.

Either the dried solids, wet filtered solids or the residual slurry obtained after evaporation of the digestion solvent may be taken up in a solvent in which the epoxy steroid product is moderately soluble, e.g., 2-butanone (methyl ethyl ketone), methanol, isopropanol-water or acetone-water. The resulting solution may typically contain between about 3% and about 20% by weight, more typically between about 5% and about 10% by weight, steroid. The resulting solution may be filtered, if desired, and then evaporated to remove the polar solvent and recrystallize the 9,11-epoxy steroid. Where the solvent is 2-butanone, evaporation is conveniently conducted at atmospheric pressure, but other pressure conditions may be used. The resulting slurry is cooled slowly to crystallize additional steroid. For example, the slurry may be cooled from the distillation temperature (about 80° C. in the case of 2-butanone at atmospheric pressure) to a temperature at which yield of steroid product is deemed satisfactory. Production of a highly pure 9,11-epoxy steroid product of a suitable crystal size may be produced by cooling in stages and holding the temperature for a period between cooling stages. An exemplary cooling schedule comprises cooling in a first stage to a temperature in the range of 60° to 70° C., cooling in a second stage to a temperature in the range of about 45° to about 55° C., cooling in a third stage to a temperature between about 30′ and about 40° C., and cooling in a final stage to a temperature between about 10′ and about 20° C., with substantially constnt temperature hold periods of 30 to 120 minutes between cooling stages.

The recrystallized product may then be recovered by filtration and dried. Dyring may be conducted effectively at near ambient temperature. The dried product may remain solvated with the polar solvent used early in the product recovery protocol, typically ethanol. Drying and desolvation may be completed at elevated temperature under pressure or vacuum, e.g., at 75° to 95° C.

Mother liquor from the recrystallization step may be recycled for use in refining the steroid product slurry obtained from evaporative removal of the epoxidation reaction solvent, as described hereinabove.

At a charge ratio of 7 moles peroxide per mole substrate in the oxidation of the Δ^{9,11} precursor to eplerenone, decomposition of the peroxide releases only about 280 liters molecular oxygen per kg eplerenone. At a charge ratio of 4 moles peroxide per mole substrate, the oxygen release is only about 160 liters/kg eplerenone. This constrasts with a release of 400 liters/kg eplerenone at a charge ratio of 10 moles peroxide per mole substrate. By way of further example, at a charge ratio of 4 moles peroxide per mole substrate, a substrate concentration of 12% in a methylene chloride solvent, a peroxide concentration in the aqueous phase of 30%, an initial reaction temperature of 30° C., substantially at atmospheric pressure under an inert gas purge, and a reactor head space volume fraction of 15%, the maximum internal pressure that can be generated in the epoxidation reactor upon exothermic decomposition of the entire peroxide charge is about 682 psig. Moreover, even in this instance, the initial exotherm is modest enough that a reasonably skilled operator should have ample time to safely deal with loss of agitation or other process upset that could otherwise potentially lead to uncontrolled reaction.

At the relatively low peroxide to substrate ratios described herein, either significantly lesser potential evolution of oxygen can be assured at the same reactor payload that can be achieved at peroxide/substrate ratios of 10 or more; or higher reactor payloads may be achieved at the same volume of oxygen release. At constant working volume in an epoxidation reactor, both an increase in payload and a reduction in oxygen release can be achieved.

Reaction Scheme II, as depicted below, proceeds in the same manner as Reaction Scheme I through the preparation of a compound of the Formula XXII. Then instead of proceeding with carbonylation, the process contacts the compound of Formula XXII with an oxidizing agent such as DDQ or chloranil to effect the 6,7-dehydrogenation and produce a 3-keto steroid compound of Formula XXV 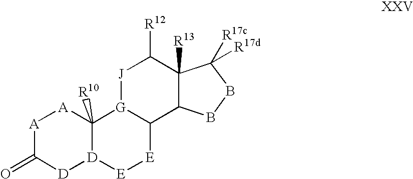
wherein R¹⁰, R¹², R¹³, -A-A-, and —B—B— are as defined above for Formula XXI; R^{17c} and R^{17d} are as defined above for Formula XXII; and -D-D-, -G-J-, and -E-E- are as defined above for Formula XXVIII.

Preferably, the compound of Formula XXII is a compound of Formula XXIIA, as shown above in Scheme I, and the compound of Formula XXV is a compound of Formula XXVA 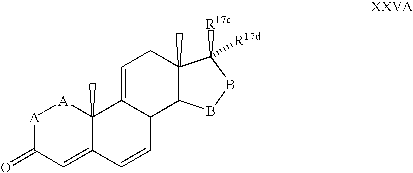
wherein
-A-A- represents the group —CH₂—CH₂— or —CH═CH—;
—B—B— represents the group —CH₂—CH₂— or an α- or β-oriented group: 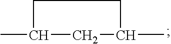
   and
R^{17c} is hydroxy or protected hydroxy; and
R^{17d} is alkenyl.

In a particularly preferred embodiment, the compound of Formula XXII is vinyl 2DM, as shown above and the compound of Formula XXV is a compound of Formula B, shown above in Table 1.

The compound of Formula XXV is then carbonylated to yield a compound of Formula XXVIII, as shown above in Scheme I. Preferably, the compound of Formula XXV is a compound of Formula XXVA as shown above, and the compound of Formula XXVIII is a compound of Formula XXVIIIA, as shown above in Scheme I. In a particularly preferred embodiment, the compound of Formula XXV is a compound of Formula B, as shown above in Table 1, and the compound of Formula XXVIII is Δ⁹⁽¹¹⁾-canrenone.

Furylation, oxidation of the furyl intermediate to the 7α-carboxylic acid, esterification to the 7α-methyl ester, and epoxidation to epoxymexrenone then follow according to the same sequence as in Reaction Scheme I. Reaction Scheme II is advantageous in avoiding the carbonylation of the vinyl 2DM structure, which can be attended by some unwanted conversion of the 3-methyl enol ether with formation of Δ⁹⁽¹¹⁾-aldona. A reducing agent such as hydrogen is preferably present to promote the carbonylation. Optionally, an acidic reducing agent may be present, such as formic acid, oxalic acid, or phosphinic acid. Because the 3-methyl enol ether can be degraded in the presence of acid, especially where water is also present as is typically the case with formic or other protic acids, hydrogen is generally preferred as the reducing agent. However, in the process of Scheme II, hydrolysis of the 3-alkyl enol ether is avoided by reversing the sequence of 6-7-dehydrogenation and carbonylation, thereby facilitating the use of an acidic reducing agent if otherwise desired. Eliminating restrictions on acidity in the carbonylation allows this step to be conducted without the presence of hydrogen.

Carbonylation of 3-keto-triene per Reaction Scheme II can result in some reduction of the 6,7-double bond formed during the preceding step. Either the solvent used in the preceding 6,7-dehydrogenation or in the subsequent furylation may be used in the carbonylation step of Reaction Scheme II. Solvents such as dioxane or tetrahydrofuran are among the wide range of suitable choices. DEPhos is preferrably used as the catalyst ligand. The molar ratio of DEPhos or other ligand to Pd(OAc)₂ is preferrably maintained in range between abouit 1:1 and about 3:1 or slightly greater, preferably in the neighborhood of 2:1, and the temperature is preferrably maintained at at least about 90° C., more preferably at least about 100° C. Operation under such conditions has been found to afford substantially 100% conversion at a steroid concentration of 15% to 20%, with formation of less than 2% Δ⁹⁽¹¹⁾-aldona based on the 3-keto-triene charge.

Reaction Scheme III, as further depicted below, differs from Reaction Scheme II in reversing the sequence of carbonylation and furylation. Thus, both 6,7-dehydrogenation and furylation intervene between the alkynyl hydrogenation and vinyl carbonylation steps.

In Scheme III, the compound of Formula XXV is furylated to produced a compound of Formula XXVII: 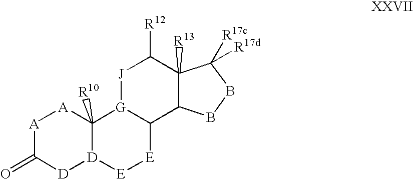
wherein R¹⁰, R¹², R¹³, -A-A-, and —B—B— are as defined above for Formula XXI; R^{17c} and R^{17d} are as defined above for Formula XXII; and -D-D-, -G-J-, and -E-E- are as defined above for Formula XXVIII.

Preferably, the compound of Formula XXV is a compound of Formula XXVA, as shown above, and the compound of Formula XXVII is a compound of Formula XXVIIA: 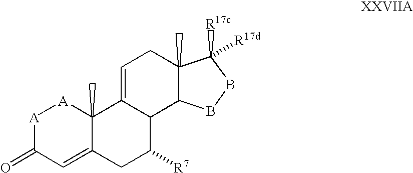
wherein
-A-A- represents the group —CH₂—CH₂— or —CH═CH—;
—B—B— represents the group —CH₂—CH₂— or an α- or β-oriented group: 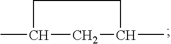
R⁷ is selected from the group consisting of hydrogen, furyl, and alkylfuryl;
R^{17c} is hydroxy or protected hydroxy; and
R^{17d} is alkenyl.

Even more preferably, the compound of Formula XXV is a compound of Formula B and the compound of Formula XXVII is a compound of Formula E; the structures of Formulae B and E are shown above in Table 1.

The compound of Formula XXVII is then carbonylated to produce a compound of Formula XXIX, as shown above.

Preferably, the compound of Formula XXVII is a compound of Formula XXVIIA, as shown above, and the compound of Formula XXIX is a compound of Formula XXIXA, as shown above.

Even more preferably, the compound of Formula XXVII is a compound of Formula E, as shown above, and the compound of Formula XXIX is:

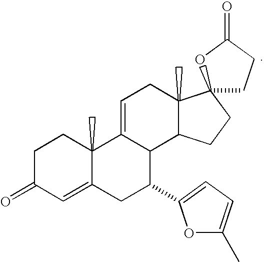

Thus, Scheme III avoids carbonylation of the triene, obviating any problem of 6,7-reduction during carbonylation. The 7α-furyl steroid is converted to epoxymexrenone in the same manner as in Schemes I and II.

Carbonylation of the triene is also avoided in Reaction Scheme IV, further depicted below. Scheme IV differs from Schemes I to III in starting with the 6,7-dehydrogenation, followed by semi-hydrogenation to convert the 17-alkynyl to the 17-alkenyl, 7-furylation, and carbonylation to form the spirolactone ring.

Thus, in Scheme IV, the compound of Formula XXI is dehydrogenated with an oxidizing agent such as DDQ or chloranil to produce a compound of Formula XXIV: 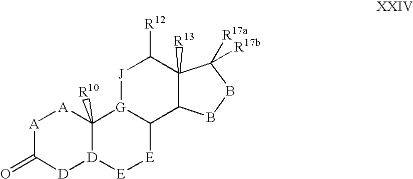
wherein R¹⁰, R¹², R¹³, R^{17a}, R^{17b}, -A-A- and —B—B— are as defined above for Formula XXI; and -D-D-, -G-J-, and -E-E- are as defined above for Formula XXVIII.

Preferably, the compound of Formula XXI is a compound of Formula XXIA, as shown above, and the compound of Formula XXIV is a compound of Formula XXIVA: 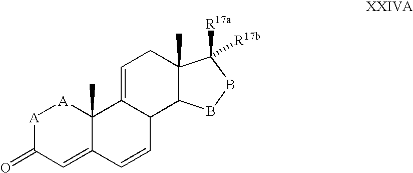
wherein
-A-A- represents the group —CH₂—CH₂— or —CH═CH—;
—B—B— represents the group —CH₂—CH₂— or an α- or β-oriented group: 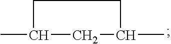
   and
R^{17a} is hydroxy or protected hydroxy; and
R^{17b} is alkynyl.

Even more preferably, the compound of Formula XXI is ethynyl 2DM, as shown above, and the compound of Formula XXIV is a compound of Formula C, as shown above in Table 1.

The 6,7-unsaturated steroid of Formula XXIV is then semi-hydrogenated to produce the intermediate of Formula XXV. Preferably, the compound of Formula XXIV is a compound of Formula XXIVA, and the compound of Formula XXV is a compound of Formula XXVA. Even more preferably, the compound of Formula XXIV is a compound of Formula C, and the compound of Formula XXV is a compound of Formula B.

The synthesis of Scheme IV then proceeds to epoxymexrenone on the same path as Scheme III.

Reaction Scheme V, also depicted below, starts with the 6,7-dehydrogenation of the 17-alkynyl intermediate, but then conducts the hydrogenation and carbonylation in immediate sequence, similar in this regard to Scheme I. Scheme V is the same as Scheme IV through the preparation of the compound of Formula XXV. According to Scheme V, this intermediate is then carbonylated to give a compound of Formula XXVIII as shown above in Scheme I. This intermediate may then be converted to the final product in the same manner as in Scheme I.

Reaction Scheme VI, further depicted below, is the same as Schemes IV and V through the preparation of the intermediate of Formula XXIV. In Scheme VI, this intermediate is then furylated to produce a compound of XXVI: 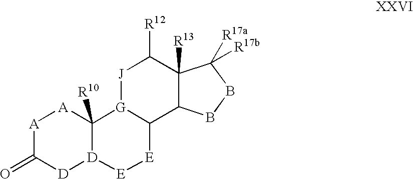
wherein R¹⁰, R¹², R¹³, R^{17a}, R^{17b}, -A-A-, and —B—B— are as defined above for Formula XXI; and -D-D-, -G-J-, and -E-E- are as defined above for Formula XXVIII.

In reaction scheme VII, 2DM is first subjected to 6,7-dehydrogenation rather than ethynylation, thereby producing Δ-4(5),6(7),9(11)-androstene-3,17-dione designated Compound XXXIII. Furylation of compound XXIII produces the 7α-furyl derivative, i.e., compound XXXIV. Ethynylation of compound XXXIV yields the 17-β-hydroxy-17α-ethynyl derivative (compound XXVI), which is then semi-hydrogenated to the 17-β-hydroxy-17α-vinyl intermediate (compound XXVII). Carbonylation yields compound XXIX, which is then converted to eplerenone in the same as described above for Schemes I to VI. The conditions for 6,7-hydrogenation, 7-furylation, ethynylation, hydrogenation, carbonylation and conversion of 7α-furyl intermediate (compound XXIX) to eplerenone are substantially as described above.

Scheme VII is potentially advantageous in moving what can be a relatively low yield step, i.e., the furylation to a point early in the process, thus minimizing the consumption of relatively expensive intermediates as produced downstream of that step.

Preferably, the compound of Formula XXIV is a compound of Formula XXIVA, as shown above, and the compound of Formula XXVI is a compound of Formula XXVIA: 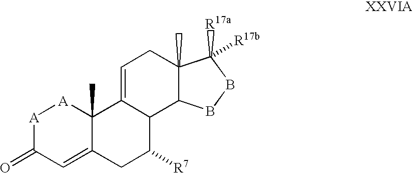
wherein
-A-A- represents the group —CH₂—CH₂— or —CH═CH—;
—B—B— represents the group —CH₂—CH₂— or an α- or β-oriented group: 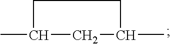
R⁷ is selected from the group consisting of hydrogen, furyl, and alkylfuryl;
R^{17a} is hydroxy or protected hydroxy; and
R^{17b} is alkynyl.

Even more preferably, the compound of Formula XXIV is a compound of Formula C, as shown above, and the compound of Formula XXVI is a compound of Formula D, shown above in Table 1.

The intermediate of Formula XXVI is then semi-hydrogenated to give a compound of Formula XXVII, as shown above in Scheme III, and the Scheme proceeds as shown in Scheme III. Because Scheme VI requires the furylation to be conducted ahead of both the ethynyl hydrogenation and carbonylation steps, this Scheme likewise avoids possible 6,7-reduction during the carbonylation.

The 17-hydroxy-17-vinyl-3-alkyl enol ether intermediate of Formula A, produced in Schemes I, II, and III, the 17-hydroxy-17-vinyl-3-keto intermediate of Formula B, produced in Schemes II, III, IV and VI, the 17-hydroxy-17-ethynyl-3-keto intermediate of Formula C, produced in Schemes IV and VI, the 17-hydroxy-17-ethynyl-3-keto-7α-methylfuryl intermediate of Formula D, produced in Schemes V and VI, and the 17-hydroxy-17-vinyl-3-keto-7α-methylfuryl intermediate of Formula E, produced in Schemes IV, V and VI, are all novel compounds, each highly useful in the preparation of epoxymexrenone.

Moreover, other compounds having structures corresponding to Formulae A, B, C, D and E are also novel and useful for the same purpose. Thus, the novel compounds of the invention comprise compounds corresponding to Formulae XXII, XXIV, XXV, XXVI, and XXVII, as defined above and in the claims as appended hereto. Novel species of the various compounds of this invention are set forth below:

TABLE I

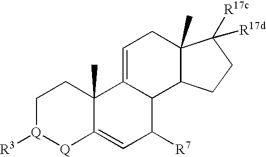

| Compound | —QR³—Q— | R^{17c} | R^{17d} | R⁷ |
|---|---|---|---|---|
| 22-1 | 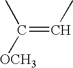 | OH | vinyl | H |
| 22-1a | 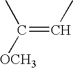 | β-OH | α-vinyl | H |
| 22-2 | 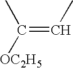 | OH | vinyl | H |
| 22-2a | 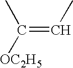 | β-OH | α-vinyl | H |
| 22-3 | 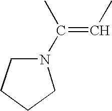 | OH | vinyl | H |
| 22-3a | 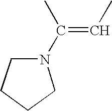 | β-OH | α-vinyl | H |
| 22-4 | 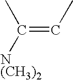 | OH | vinyl | H |
| 22-4a | 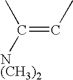 | β-OH | α-vinyl | H |
| 22-5 | 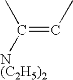 | OH | vinyl | H |
| 22-5a | 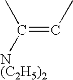 | β-OH | α-vinyl | H |
| 22-6 | 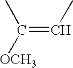 | OH | vinyl | 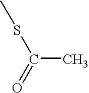 |
| 22-6a | 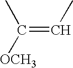 | α-OH | β-vinyl | 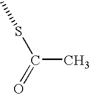 |
| 22-6b | 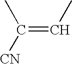 | OH | vinyl | 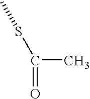 |
| 22-7 | 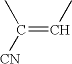 | OH | vinyl | H |
| 22-7a | 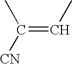 | α-OH | β-vinyl | H |
| 22-8 | 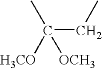 | OH | vinyl | H |
| 22-8a | 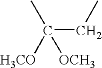 | β-OH | α-vinyl | H |
| 22-9 | 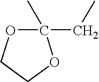 | OH | vinyl | H |
| 22-9a | 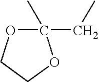 | β-OH | α-vinyl | H |
| 22-10 | 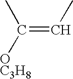 | OH | vinyl | H |
| 22-10a | 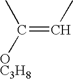 | β-OH | α-vinyl | H |
| 22-11 | 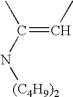 | OH | vinyl | H |
| 22-11a | 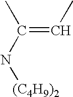 | β-OH | α-vinyl | H |

TABLE II

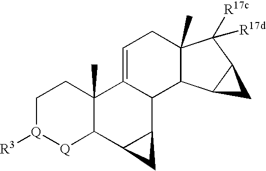

| Compound | _QR³_α_ | R^{17c} | R^{17d} |
|---|---|---|---|
| 22000 | 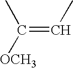 | OH | vinyl |
| 22000a | 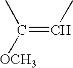 | β-OH | α-vinyl |
| 22001 | 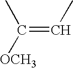 | OH | vinyl |
| 22001a | 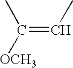 | β-OH | α-vinyl |
| 22002 | 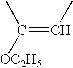 | OH | vinyl |
| 22002a | 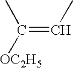 | β-OH | α-vinyl |
| 22003 | 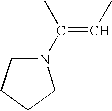 | OH | vinyl |
| 22003a | 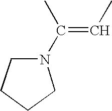 | β-OH | α-vinyl |
| 22004 | 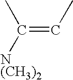 | OH | vinyl |
| 22004a | 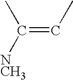 | β-OH | α-vinyl |
| 22005 | 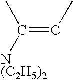 | OH | vinyl |
| 22005a | 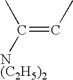 | β-OH | α-vinyl |
| 22006 | 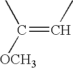 | OH | vinyl |
| 22006a | 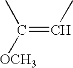 | α-OH | β-vinyl |
| 22006b | 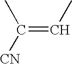 | OH | vinyl |
| 22007 | 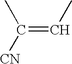 | OH | vinyl |
| 22007a | 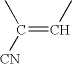 | α-OH | β-vinyl |
| 22008 | 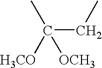 | OH | vinyl |
| 22008a | 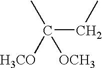 | β-OH | α-vinyl |
| 22009 | 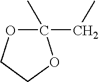 | OH | vinyl |
| 22009a | 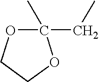 | β-OH | α-vinyl |

TABLE III

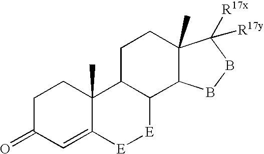

| Compound | —E—E— | —B—B— | R^{17c} | R^{17d} |
|---|---|---|---|---|
| 25-1 | —CH═CH— | —CH₂—CH₂— | OH | vinyl |
| 25-1a | —CH═CH— | —CH₂—CH₂— | β-OH | α-vinyl |
| 25-2 | 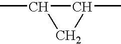 | 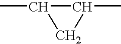 | OH | vinyl |
| 25-2a | 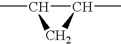 | 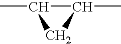 | β-OH | α-vinyl |
| 25-4 | 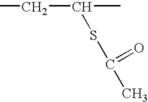 | —CH₂—CH₂— | OH | vinyl |
| 25-4a | 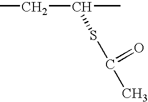 | —CH₂—CH₂— | β-OH | α-vinyl |
| 25-5 | 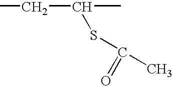 | 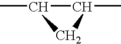 | OH | vinyl |
| 25-6a | 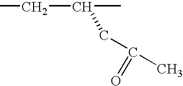 | 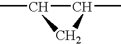 | β-OH | α-vinyl |
| 24-1 | —CH═CH— | —CH₂—CH₂— | OH | ethynyl |
| 24-1a | —CH═CH— | —CH₂—CH₂— | β-OH | α-ethynyl |
| 24-2 | 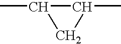 | 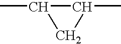 | OH | ethynyl |
| 24-2a | 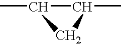 | 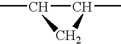 | β-OH | α-ethynyl |
| 24-4 | 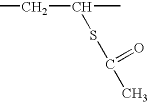 | —CH₂—CH₂— | OH | α-ethynyl |
| 24-4a | 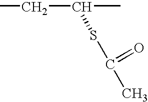 | —CH₂—CH₂— | β-OH | α-ethynyl |
| 24-5 | 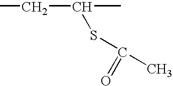 | 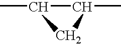 | OH | ethynyl |
| 24-5a | 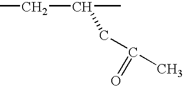 | 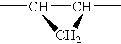 | β-OH | α-ethynyl |
| 26-1 | 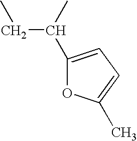 | —CH₂—CH₂— | OH | ethynyl |
| 26-1a | 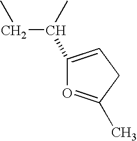 | —CH₂—CH₂— | β-OH | α-ethynyl |
| 27-1 | 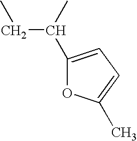 | —CH₂—CH₂— | OH | vinyl |
| 27-1a | 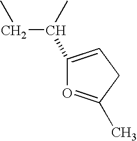 | —CH₂—CH₂— | β-OH | α-vinyl |

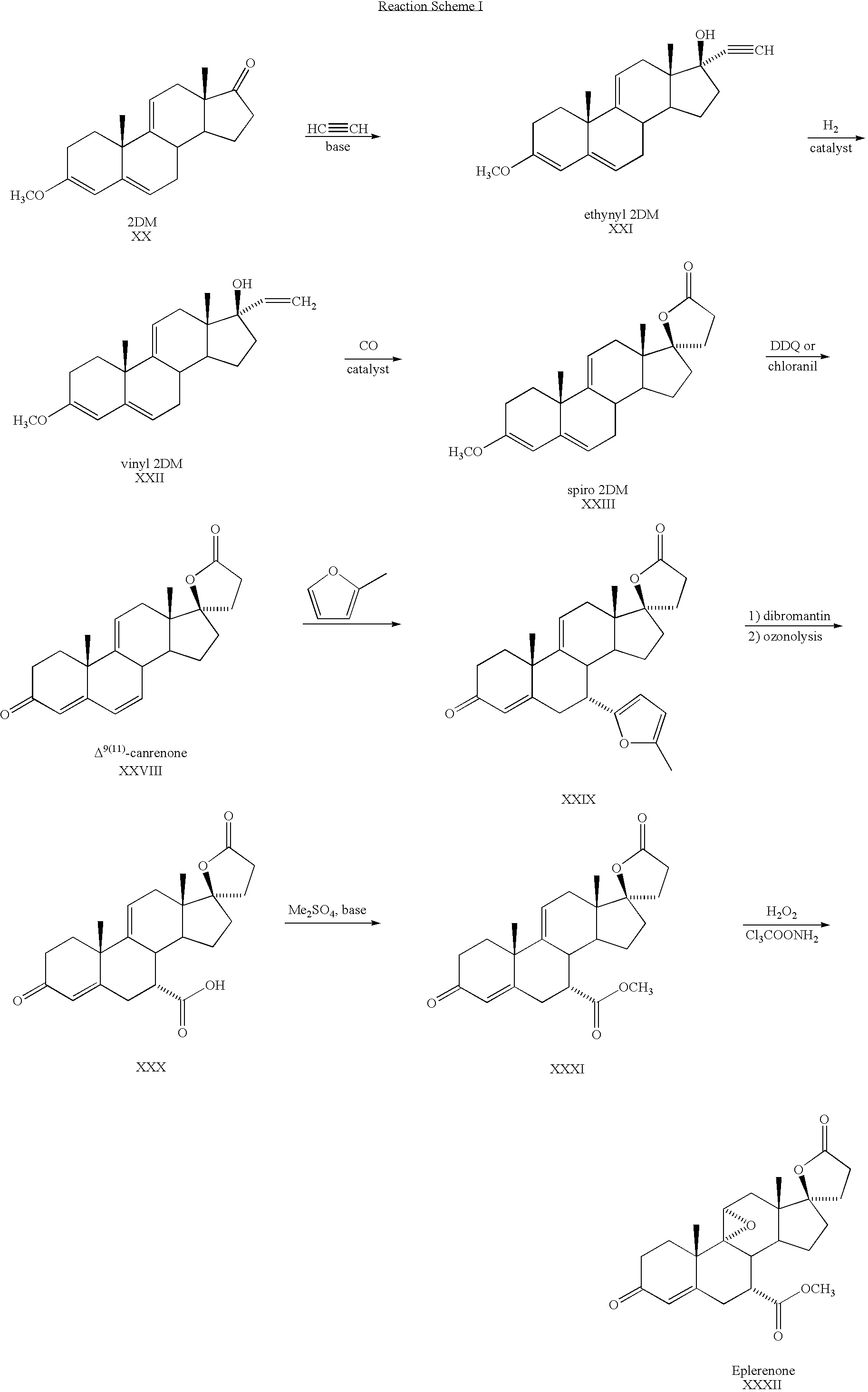

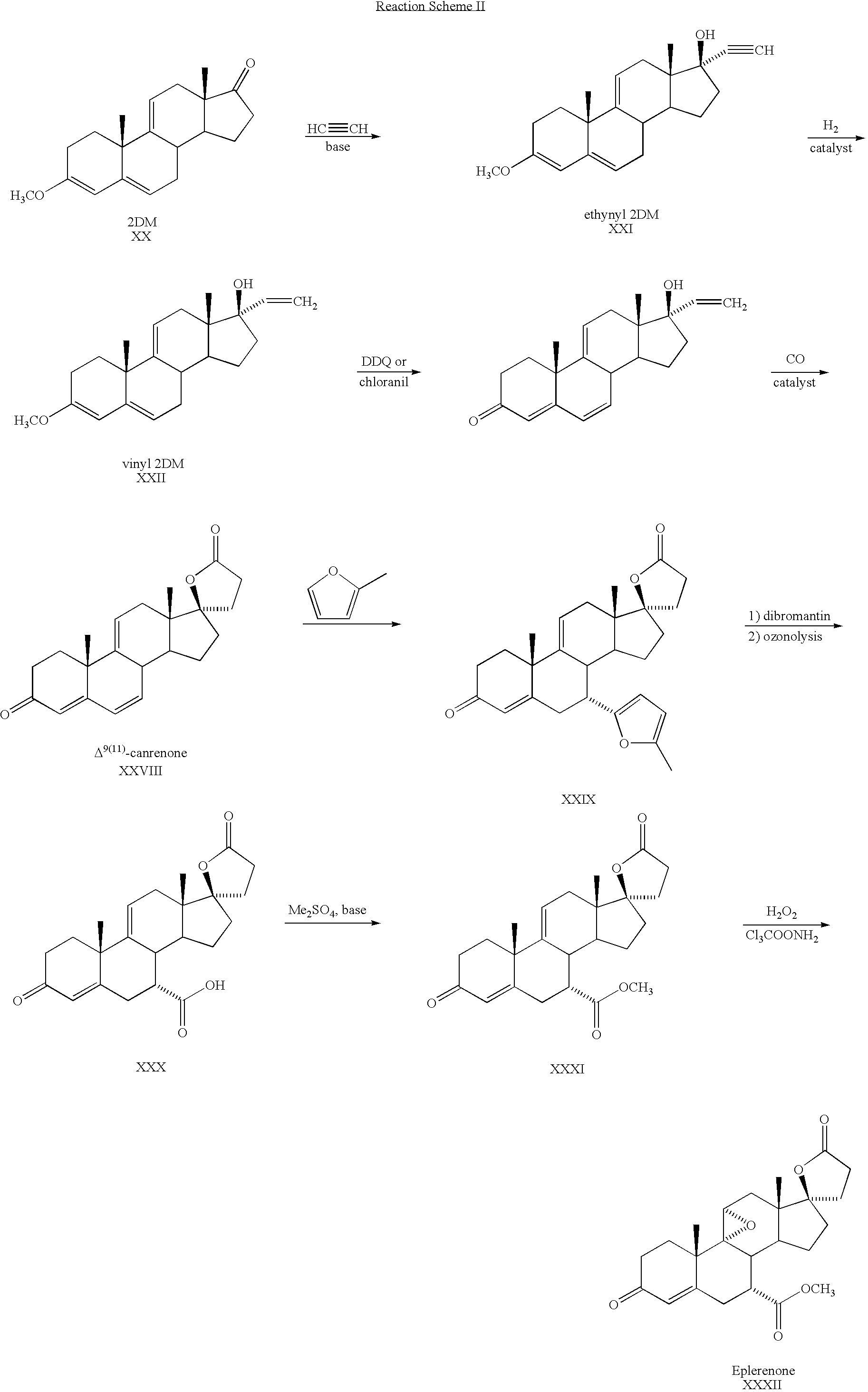

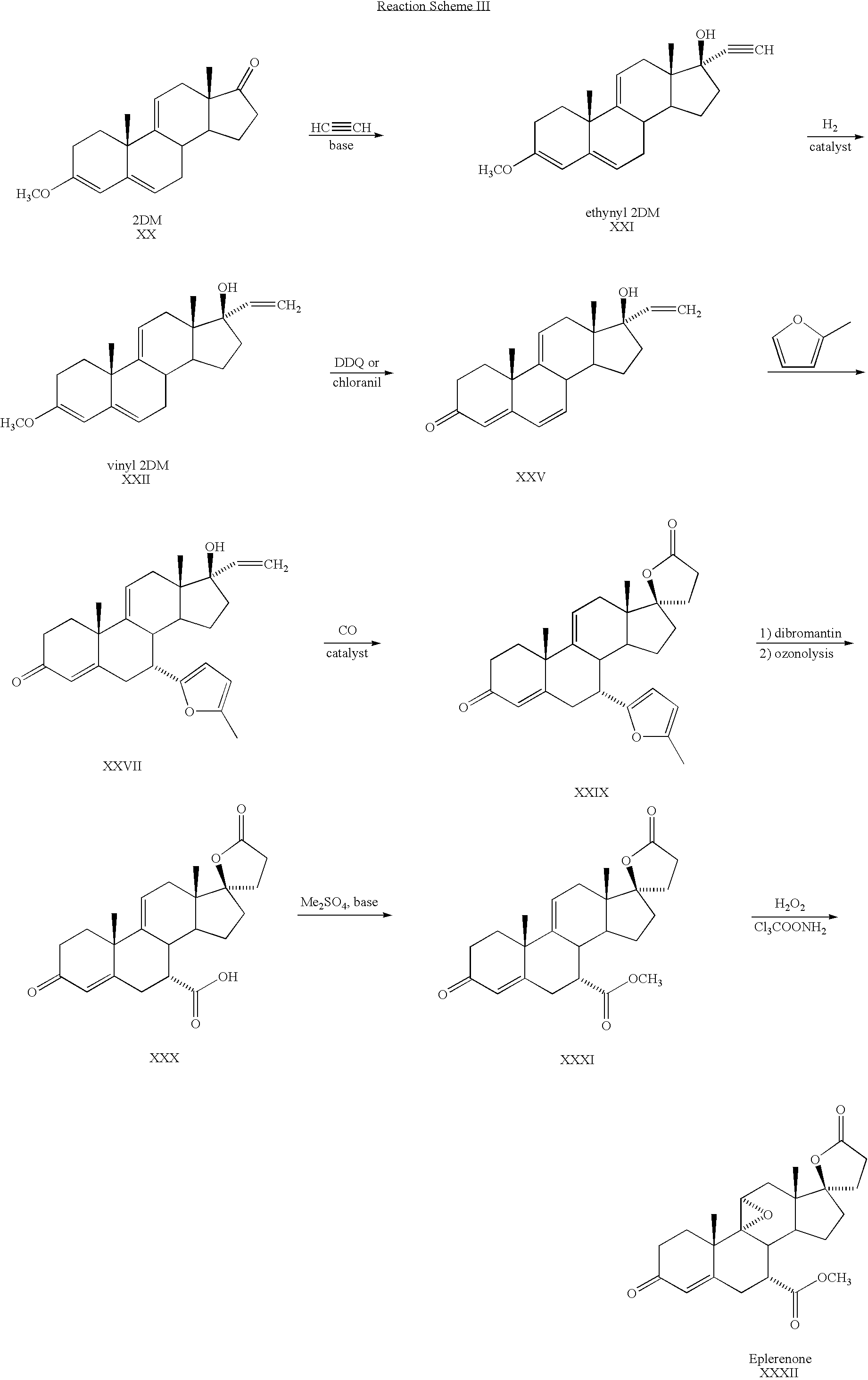

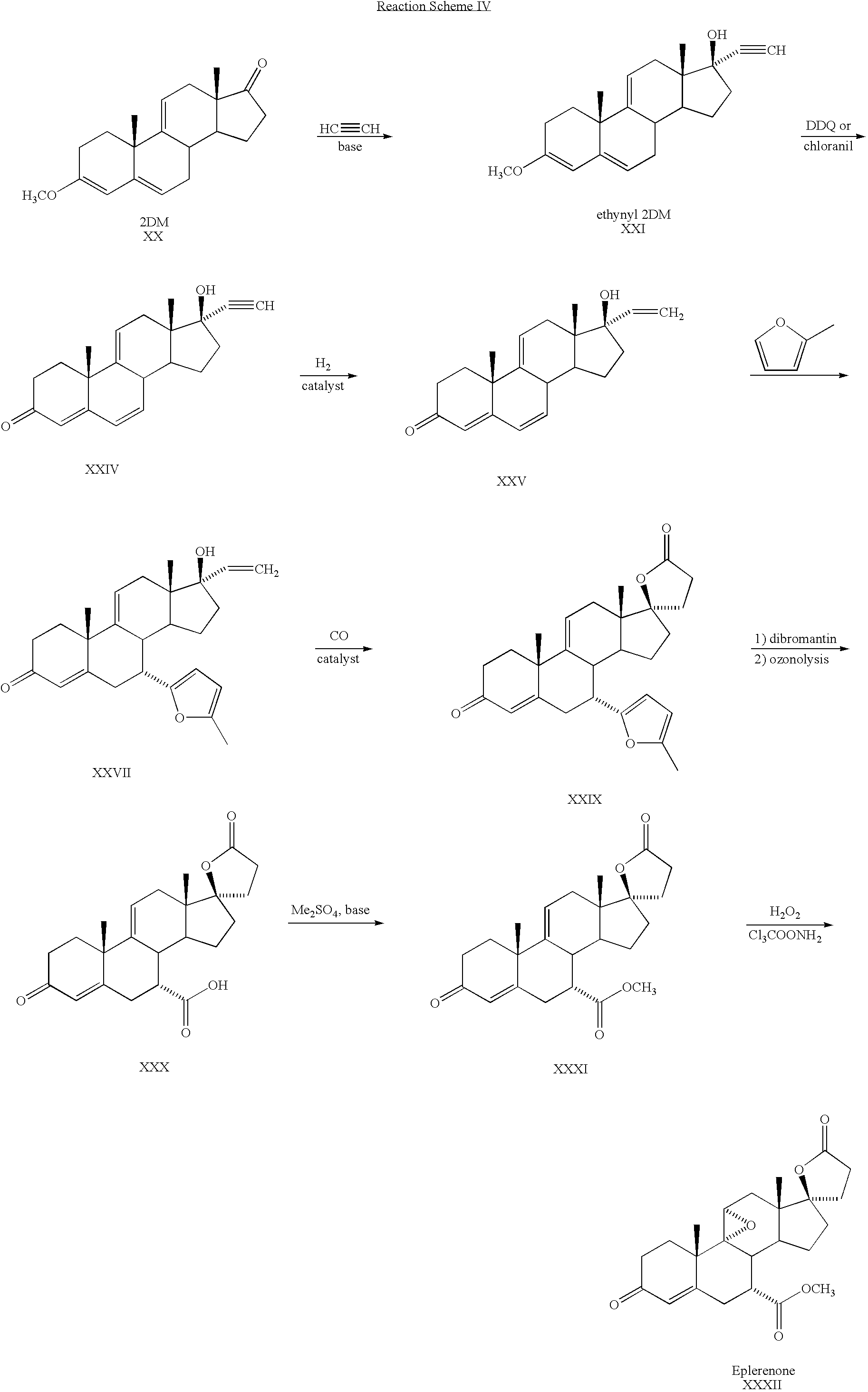

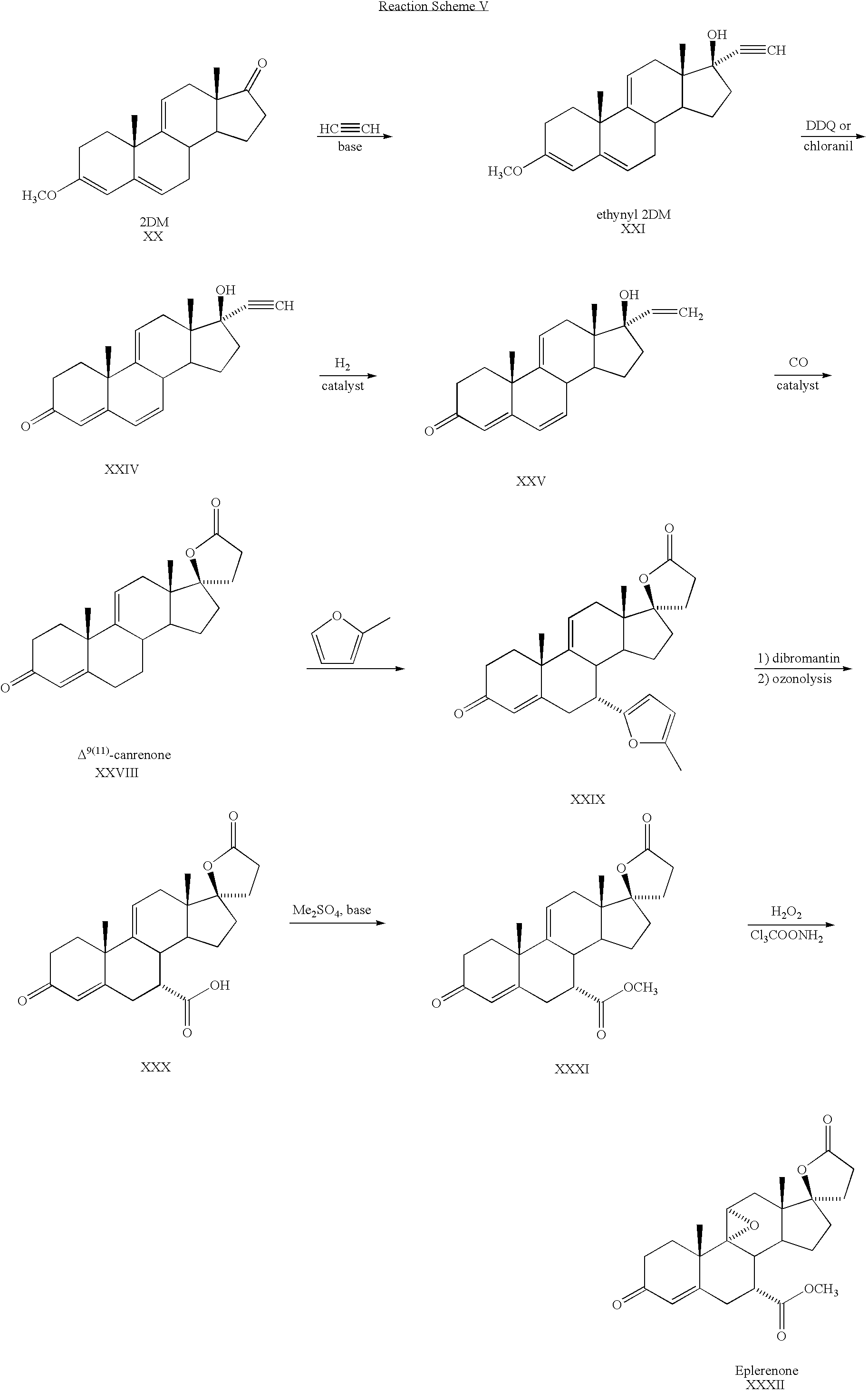

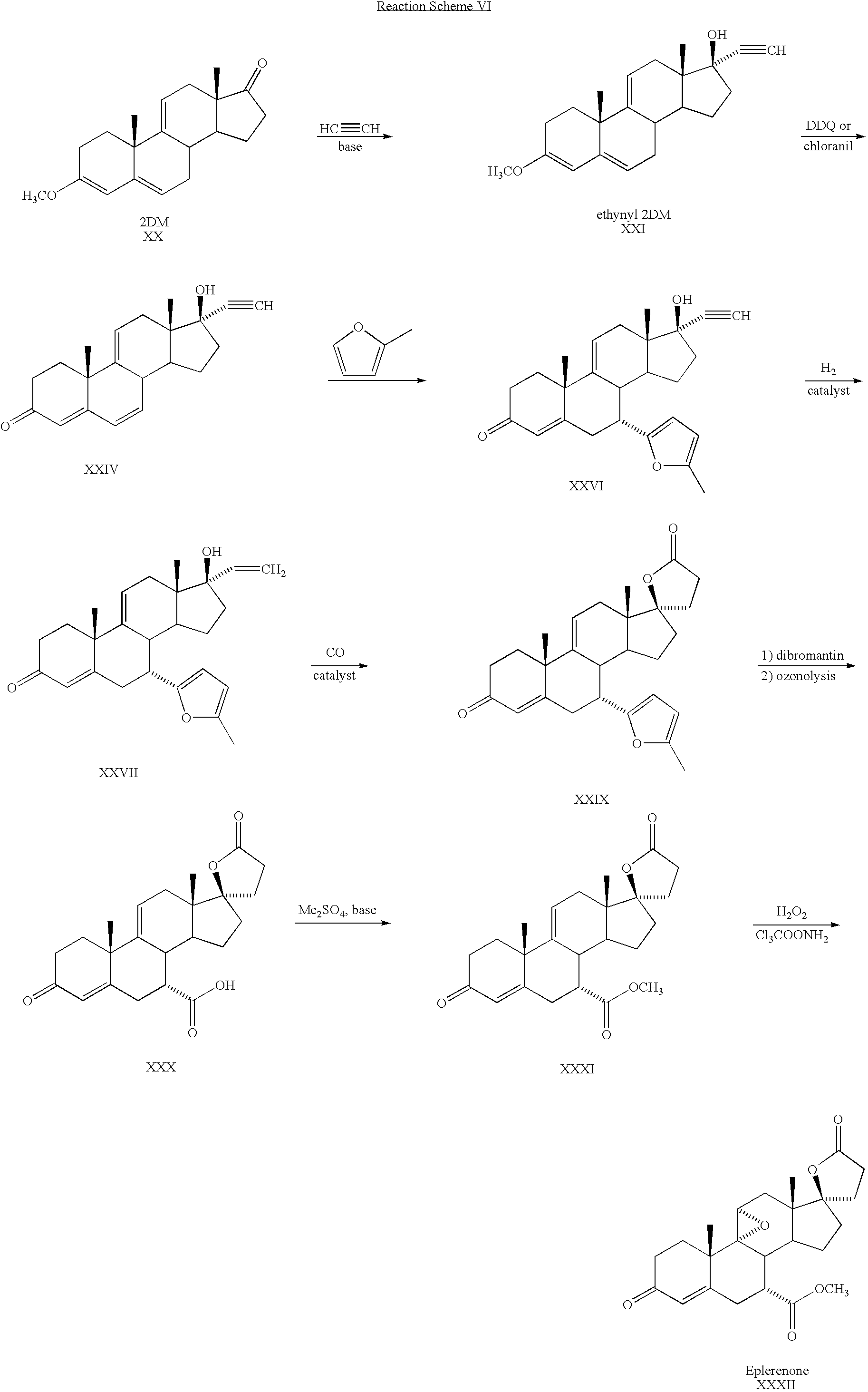

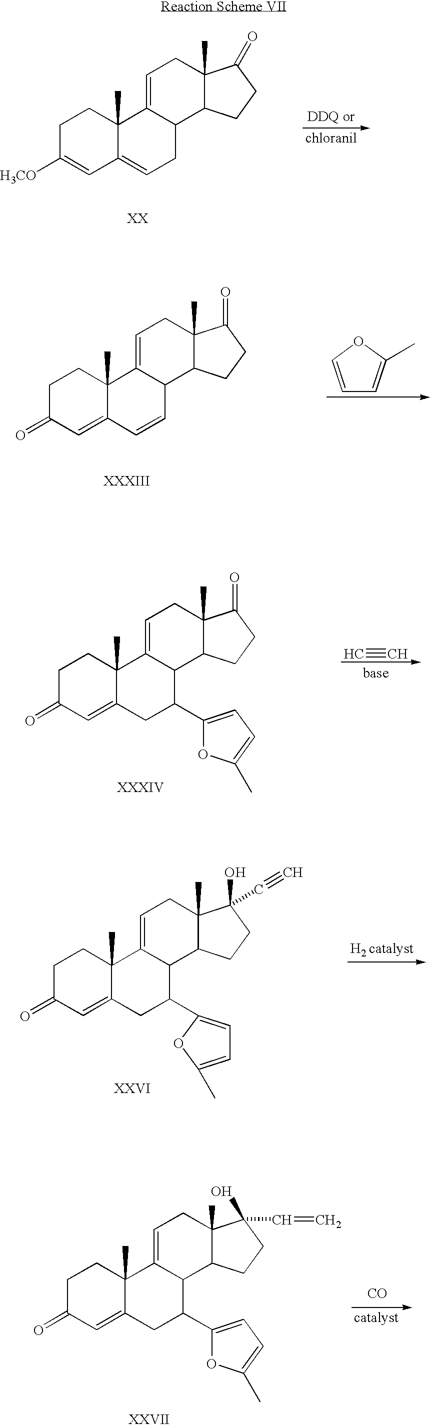

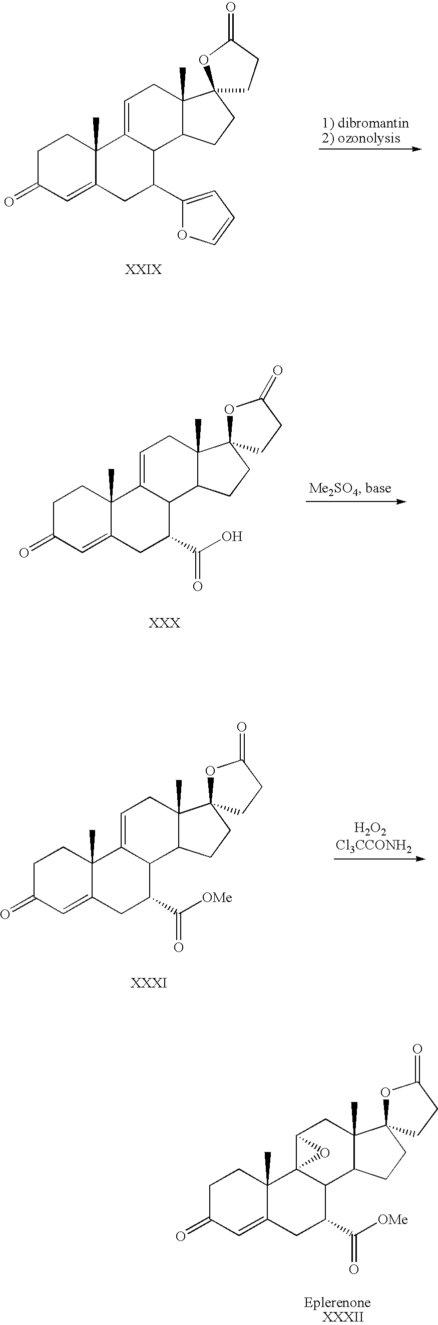

The carbonylation process of the invention may also be used in the preparation of drospirenone and analogs thereof. For example, the 3,5-β-dihydroxy-6,7-β-15,16β-dimethylene-17-keto steroid corresponding to the formula D101:

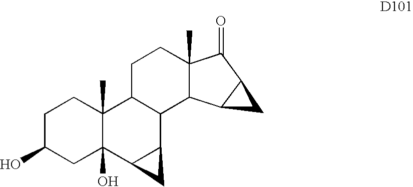

May be ethynylated as described herein, e.g., by reaction with acetylene and base, to produce the corresponding 3,5-β-dihydroxy-6,7-β-15,16β-dimethylene-17-β-hydroxy-17-α-ethynyl intermediate D102:

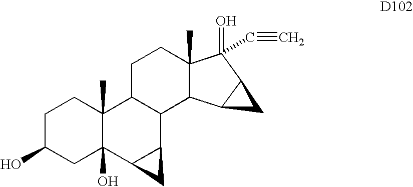

A 17-spirolactone group may then be introduced as described herein, either by sequential semi-hydrogenation to the 17β-hydroxy-17-α-vinyl intermediate D103: 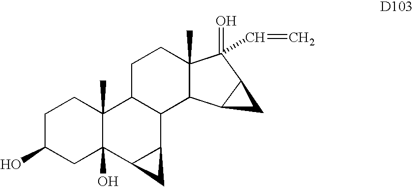
followed by carbonylation to form the lactone ring, or by in situ hydrogenation and carbonylation, or by direct carbonylation. This yields the 3-β, 5-β-dihydroxy-6,7-β, 15,16-β-dimethylene-17-spirobutyrolactone intermediate D104:

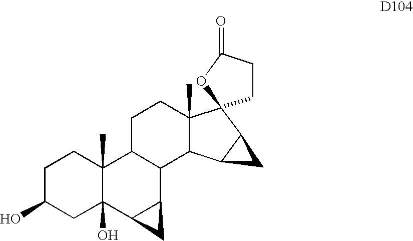

The 3-β-hydroxy of D104 may be oxidized to the corresponding 3-keto derivative in any conventional manner, e.g., by reaction with an oxidizing agent such as pyridinium chromate in DMF, as described in U.S. Pat. No. 6,121,465, expressly incorporated herein by reference, to produce a mixture of drospirenone and the 5-β-hydroxy intermediate denominated therein as 5-β-DRSP:

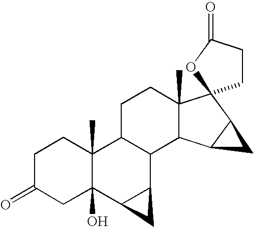

The latter may be converted to drospirenone by dehydration with HCl as further described in EP 1149840:

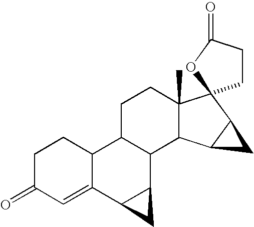

It will be understood, that the above synthesis encompasses derivatives of drospirenone and intermediates thereof wherein the steroid structure may have other structures or substituents at C-1, C-2 (as defined with respect to -A-A-), C-9, C-11 and C-12 (as defined with respect to R⁹, R¹¹ and R¹², respectively).

### EXAMPLES

The following examples are simply intended to further illustrate and explain the present invention. This invention, therefore, should not be limited to any of the details in these examples.

### Example 1

### Preparation of Ethynyl 2DM from 2DM

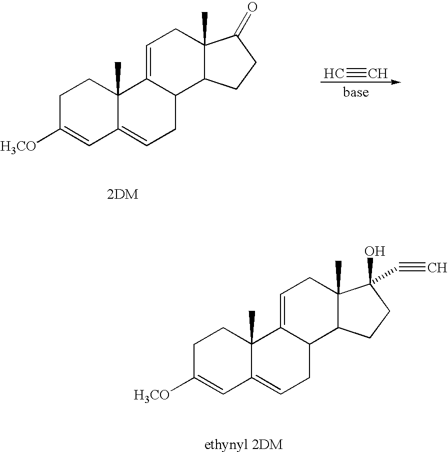

To a nitrogen-purged 5000-mL flask was charged 2DM (300 g) and about 1800 mL BHT-stabilized THF. The slurry was cooled to −13° C. and 1410 g of 20% by weight potassium t-butoxide in THF (1550 mL) was added. The homogeneous solution was stirred at −10° to −15° C. while acetylene gas is introduced subsurface at a rate of about 5 cubic feet per hour until the reaction was essentially complete by TLC (about 3 hours). The reaction was carefully quenched by the addition of nitrogen-sparged water (120 mL) during which time gas was evolved and the temperature of the mixture rose to about 0° C. The mixture was cooled to −5° C. and glacial acetic acid (143.4 g) and methanol (50 mL) were added. Water (about 60 mL) was then added to dissolve the solid salts resulting in the formation of two liquid phases. The lower aqueous phase was removed and discarded. The remaining upper phase was distilled under vacuum with portionwise introduction of methanol (6×500 mL) until most of the THF was removed and the final volume of the mixture was 1200 mL. Crystals began forming during the distillation process and the resulting mixture was cooled to 0° C. The crystals were then collected by filtration, washed with cold methanol (500 mL), and dried at 25° C. under a stream of nitrogen to afford 287.1 g (88.0%) of ethynyl 2DM that was 99.4% pure by HPLC.

### Example 2

### Scheme I, Step 2: Selective Hydrogenation of Ethynyl 2DM to Vinyl 2DM

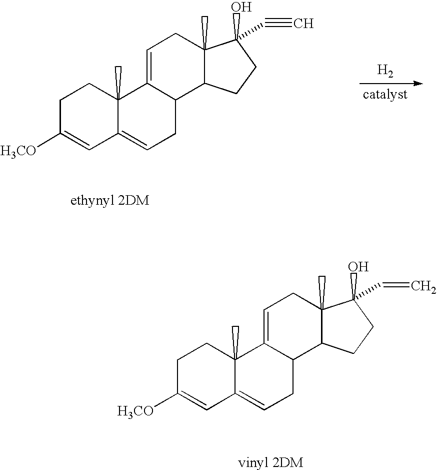

Ethynyl 2DM (25.0 g), methanol (100 mL), triethylamine (0.1 g), and Lindlar catalyst (0.0689 g, Johnson Matthey) were charged to a 300-mL stainless steel Parr autoclave equipped with a standard, four-bladed axial impeller. The reactor was sealed, purged with nitrogen followed by hydrogen, and then pressurized to 40 psig with hydrogen. The mixture was stirred (700 rpm) and heated to 50° C. Hydrogen was fed on demand from a high pressure reservoir of known volume to maintain the total pressure of the reactor at 40 psig. Hydrogen uptake was monitored by following the pressure drop in the hydrogen reservoir. After 1.1 hours, hydrogen uptake ceased. The reactor was vented and the vessel was purged with nitrogen. The reactor contents were vacuum filtered through a fine sintered glass filter using methanol to produce a filtrate (129 g) which contained no ethynyl 2DM starting material and had a ratio of vinyl 2DM to ethyl 2DM of 97.1 to 2.9.

### Example 2A

### Reduction of 17-ethynyl Testosterone to 17-vinyl Testosterone

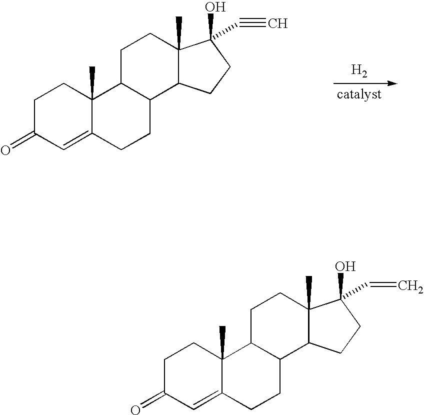

A mixture of ethisterone (50.0 g), Lindlar catalyst (Johnson Matthey, 5% Pd, 0.50 g), methanol (100 g) and 1-hexene (28.5 g) was heated in a 300-mL stirred autoclave under hydrogen (25 psig) for 4.5 hours at 40° C. followed by another 1 hour at 60° C. After cooling, the filtered crude product mixture was found to contain 98.3% 17-vinyl testosterone and 1.1% 17-ethyl testosterone by HPLC. Portionwise addition of water (2 volumes) afforded, after drying, 48.84 g of 17-vinyl testosterone 96.7% chemical yield as off-white crystals.

### Example 3

### Scheme IV, Step 3: Selective Hydrogenation of 3-keto-Δ-4(5),6(7),9(11)-ethisterone to 3-keto-Δ-4(5),6(7),9(11)-17-vinyl Testosterone

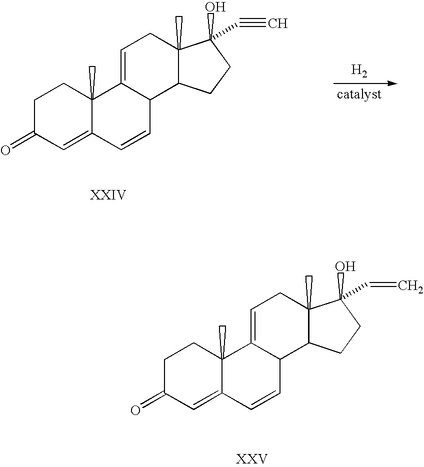

A 50-mL stainless steel autoclave was charged with 3-keto-17-ethynyl substrate (5.00 g), dichloromethane (20 mL), triethylamine (3 drops), and Lindlar catalyst (Johnson Matthey, type 310050-5, 0.0199 g). The vessel was sealed, purged first with nitrogen and then with hydrogen, and pressurized with hydrogen to 20 psig. Stirring at 400 rpm was then initiated and the reactor was fed hydrogen on-demand from a high pressure reservoir of known volume. After about 6.4 hours, gas consumption essentially ceased and the reactor was carefully vented and purged with nitrogen.

### Example 4

### Scheme VI, Step 4: Selective Hydrogenation of Δ4-(5),9(11),17-ethynyl-7α-methylfuryl Substrate

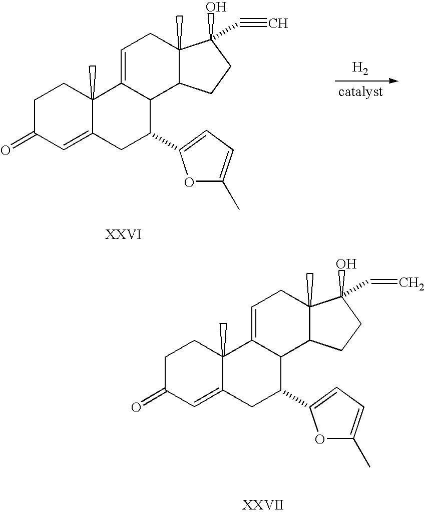

17-ethynyl-7α-methylfuryl steroid (24.65 g), Lindlar catalyst (Johnson Matthey A310050-5, 0.0871 g), and acetonitrile (100 mL) were charged to a 300-mL autoclave. The vessel was purged with nitrogen followed by hydrogen and then pressurized to 25 psig with H₂. The mixture was heated to a temperature of 50° C. while pressure was maintained at 25 psig with H₂. When the hydrogen uptake subsided, the reactor pressure was increased to 40 psig, the reaction temperature was increased to 60° C. Additions of Lindlar catalyst (up to 0.4526 g total catalyst present) were required to achieve theoretical hydrogen consumption after about 8 hours. The reaction mixture was then vacuum filtered through a sintered glass filter (fine porosity) with minimal acetonitrile addition to wash the vessel and catalyst. Filtration resulted in 118 g of an acetonitrile solution. HPLC analysis of revealed ca. 98.8% conversion (1.2% starting material) and 99.1% selectivity (0.9 area % of the 17-ethyl as a result of over-reduction).

### Example 5

### Scheme I, Step 3: Carbonylation of Vinyl 2DM

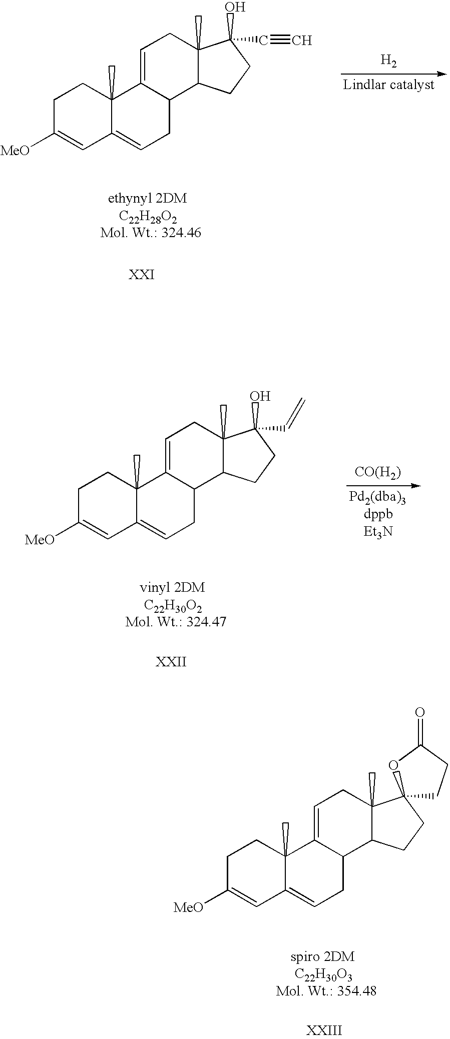 Procedure (notebook refs. 12502-121 & 12878-163): A 300-mL SS stirred autoclave was charged with ethynyl 2DM 25.00 g 77.1 mmol), methylene chloride (150 mL, 197 g), and 5% Pd Lindlar catalyst (Aldrich lot 03418LI, 0.2494 g). The vessel was sealed, purged with H₂ and pressurized to 20 psig with H₂. The mixture was agitated at room temperature (22° C.) for 4.0 hr with H₂ supplied on demand to maintain 20 psig overall pressure. The mixture was filtered through a fine sintered glass filter and the recovered catalyst was washed with about 10 mL methylene chloride. HPLC analysis revealed 94.3 area % 17-vinyl 2DM and 4.5% 17-ethyl 2DM (uncorrected area at 254 nm).

The filtrate was placed in a 300-mL SS stirred autoclave and triethylamine (0.387 g, 3.82 mmol), dppb (Strem, 0.327 g , 0.767 mmol), and Pd(dpa)₂ (Alfa, 0.22 g, 0.38 mmol) were added. The sealed vessel was purged with nitrogen and then syngas (1:1 CO/H₂, 3×200 psig). The reactor was then pressurized to 300 psig with syngas and heated to 100° C. with stirring. When the temperature reached 100° C., the total pressure was increased to 400 psig with syngas. A mixture of 1:1 CO/H₂ was suppied on demand to maintain an overall pressure of 400 psig while the temperature was maintained at 100° C. for 18 hr.

After allowing to cool to 25° C. and careful venting of the reactor pressure, the mixture was filtered over a short bed of celite and washed with additional methylene chloride. HPLC analysis of this crude mixture revealed 7.3% Δ9,11-aldona, 0.4% Δ^{9,11}-17-ethyl testosterone, 88.9% spiro 2DM, and 3.4% 17-ethyl 2DM (uncorrected area at 254 nm).

The mixture was evaporated to dryness and triturated with 300 mL of methanol containing 1 mL of triethylamine with chilling to −25° C. Filtration, washing with cold methanol, and drying in vacuo at 50° C. overnight afforded 22.59 g of material that was 95.8% pure spiro 2DM, 3.1% 17-ethyl 2DM, and 1.1% Δ^{9,11}-17-ethyl testosterone by HPLC (uncorrected area at 254 nm). Overall isolated yield from ethynyl 2DM, assuming 100% purity of starting material, is 80%. The final product filtrate contains additional spiro 2DM and Δ^{9,11}-aldona that could have been recovered.

### Example 5A

### Carbonylation of Δ9(11)-17-vinyl Testosterone

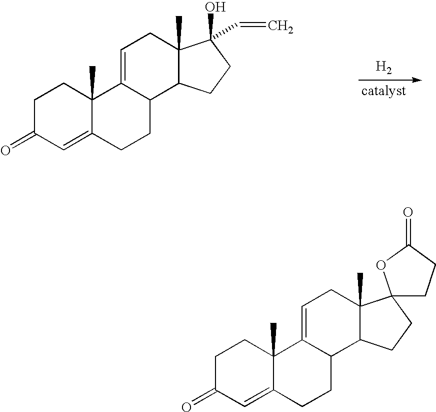

Δ9(11)-17-vinyl testosterone (25.0 g), 10% Pd/C (0.473 g comprising 0.56 mol % Pd), formic acid (7.3 g), 1,4-bis(diphenylphosphino)butane (0.949 g), PPh₃ (0.296 g) and 1,2-dimethoxyethane (163 mL) were charged to a 300-mL autoclave and the mixture was heated at a temperature of 100° C. for 18 hours under an atmosphere of carbon monoxide (100 psig). The reactor was then cooled and vented before the crude reaction product mixture was filtered through a pad of silica gel. The product mixture was then washed twice with CH₂Cl₂ and twice with acetone. Rotary evaporation of the combined filtrate afforded a thick oil. Et₂O (150 mL) was then added followed by hexanes (300 mL) to afford a white solid, which was subsequently dried. Analysis of the product showed 98.6% by weight yield of aldona.

### Example 6

### Scheme II, Step 4: Carbonylation

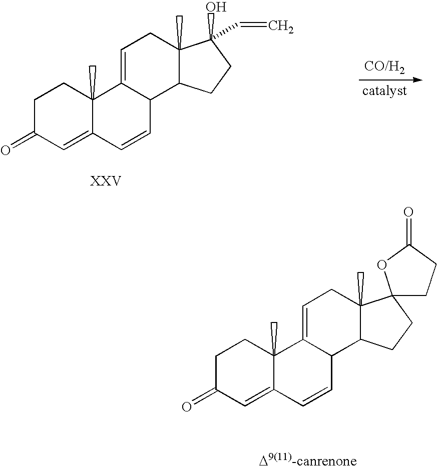

Palladium acetate (0.078 g), DPEphos (0.374 g), and the steroid substrate (crude solution as prepared in Example 3, 21.57 g) were charged to a 300-mL stainless steel reactor under argon and with agitation along with formic acid (96%, 1.6 g), and dry THF (inhibited, 98 mL). The vessel was sealed, purged with 100% CO and pressurized to about 70 psig with CO. After stirring at 25° C. for 20 minutes, the mixture was heated to 105° C. and the pressure was increased to 100 psig with CO. The reactor was fed carbon monoxide on demand from a high pressure reservoir to maintain a total pressure of 100 psig and held for 18 hours at 105° C. After cooling-and careful venting, the product mixture was filtered through a plug of silica gel (10 g) to remove some of the palladium and evaporated to dryness. The residue was dissolved in refluxing methanol (70 mL) and water (70 mL) was added dropwise with stirring. The mixture was allowed to cool to 25° C. and then placed in a freezer at −10° C. The precipitate was isolated by filtration, washed with cold 1:1 methanol/water (2×80 mL), and dried in vacuo at 70° C. overnight to afford 22.13 g (94.1% of theoretical mass) of 98.1 wt % pure Δ⁹⁽¹¹⁾-canrenone. The filtrate and washes were evaporated and dried in vacuo to afford an additional 1.55 g (6.59% of theoretical mass) of 41.1 wt % Δ⁹⁽¹¹⁾-canrenone.

### Example 7

### Scheme VI, Step 5: Carbonylation

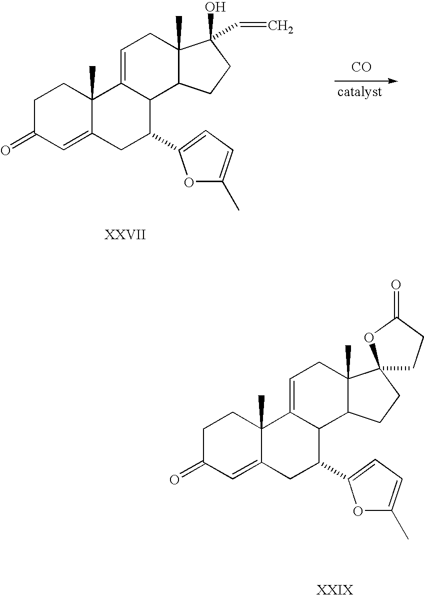

The steroid substrate prepared in Example 4 above (118 g solution containing approximately 23.15 g substrate) was transferred from the filter flask to a 300-mL stainless steel autoclave with the aid of acetonitrile (10 mL). Palladium(II) acetate (0.068 g), 96% formic acid (1.39 g) and 1,4-bis(diphenylphosphino)butane (0.257 g) were then added and the vessel was purged first with nitrogen (3×100 psig) followed by carbon monoxide (3×100 psig). The reactor was pressurized to 70 psig with CO and stirred at room temperature for 20 minutes before heating to 100° C. The system pressure was adjusted up to and maintained at 100 psig with CO as reactor reached 100° C. After 18 hours at 100° C., the reactor was cooled to room temperature and carefully vented. Filtration of the product mixture through a pad of silica gel (10 g) followed by concentration of the filtrate and acetonitrile washes to gave a crude product that was crystallized from hot acetonitrile (75 mL). After filtration, washing with cold acetonitrile, and drying in vacuo, 16.18 g (65.2% of theory) of steroid product was obtained as a white crystalline solid. Two additional crops of 4.61 g and 1.23 g of material were obtained from ethyl acetate and methanol, respectively, by successive evaporations and crystallizations of filtrates. Total yield from all three crops was 22.02 g (88.8% of theory uncorrected for assays) with an additional 1.68 g (˜6.8% of theory) of material obtained from evaporation of the final filtrate. The balance of the product (1.1 g, 4.4%) was believed to be lost during the unoptimized isolation manipulations.

### Example 8A

### Scheme I, Step 4: Preparation of (17a)-17-hydroxy-3-oxo-pregna-4,6,9(11)-triene-21-carboxylic Acid γ-lactone (i.e., Δ⁹⁽¹¹⁾-canrenone)

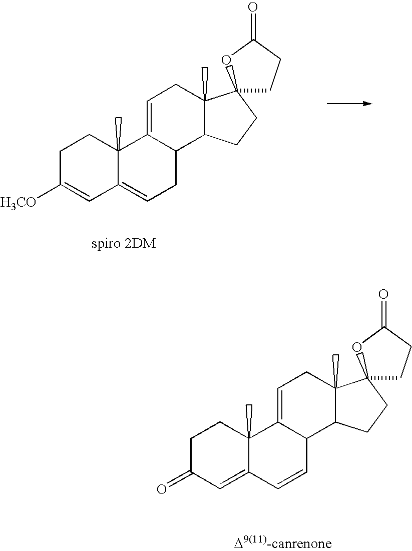

Enol ether substrate (100.0 g) and chloranil (72.2 g) were charged to a 1000-mL reactor followed by a pre-mixed solution of methylene chloride (200 mL), methanol (120 mL) and water (40 mL) while stirring. The suspension was heated to reflux (42° C.) for 2 hours over which time the mixture changed from a yellow suspension to an orange-red homogeneous solution. The reaction was checked for completion using LC. After the reaction was complete, the solution was cooled to room temperature and a solution of 20% sodium metabisulfate (30 mL) was added. The resulting mixture was stirred for 30 minutes. Water (490 mL) was added and the resulting biphase was stirred for 30 minutes. The dihydroquinone byproduct precipitated in the organic phase. The entire biphase was filtered to separate the precipitated dihydroquinone byproduct and the cake was washed twice with methylene chloride (70 mL each wash). The residual aqueous phase was removed from the filtrate and the organic phase was transferred back to the reactor for removal of the remaining dihydroquinone byproduct. The remaining byproduct was removed by contacting the residual organic phase with pulverized KOH (6.6 g) suspended in methylene chloride (70 mL) with stirring. The suspension was stirred for 1 hour and filtered to remove the dihydroquinone salt byproducts. The byproduct cake was washed twice with methylene chloride (66 mL each wash). Steroid product present in the filtrate was then isolated as described below. Prior to crystallization, the organic phase from above was washed twice with water (300 mL each wash). The mixture was then distilled at atmospheric pressure to remove methylene chloride. Methanol (379 mL) was then added and distillation was continued until the pot temperature reached 65° to 75° C. Additional methanol (35 mL) was added and the mixture was cooled to 40° C. Water (500 mL) was added over 1 hour. The suspension was then cooled within the range of 3° C. to 15° C. and held for 30 minutes. The solids were filtered and washed with a solution of methanol/water (1:1 v/v, 250 mL). Solids were dried at 70° C. in a vacuum oven with a nitrogen bleed until constant weight was obtained. Isolated 88.0 g product (92.1% molar yield unadjusted for assay).

### Example 8B

### Scheme I, Step 4: Preparation of (17α)-17-hydroxy-3-oxo-pregna-4,6,9(11)-triene-21-carboxylic Acid γ-lactone (i.e., Δ⁹⁽¹¹⁾-canrenone)

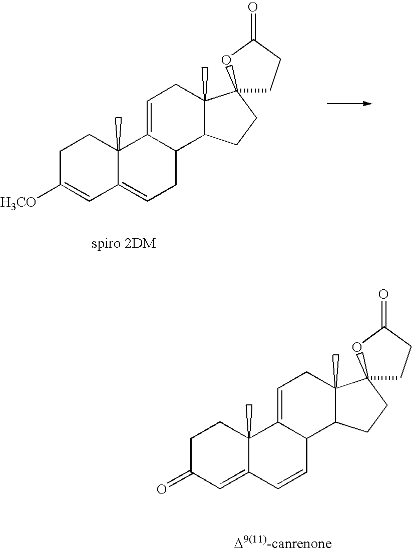

Enol ether substrate (50.1 g), acetone (200 mL) and water (50 mL) were charged to a 1000-mL, 3-necked round-bottomed flask equipped with magnetic stirring. The resulting mixture was cooled to −4° C. and 1,3-dibromo-5,5-dimethylhydantoin (22.1 g) was added in a single charge while maintaining a temperature below 10° C. The reaction was checked for completion with LC. After completion, the reaction was quenched with ethyl vinyl ether (2.5 mL). The reaction was poured onto NaHCO₃ (100 mL of ½ sat. aq. solution) and ethyl acetate (150 mL) was added. The biphase was separated and the aqueous layer was extracted with ethyl acetate (100 mL). The organic phases were combined and washed twice with water (200 mL each wash). The solution was concentrated to approximately 100 g. DMF (25 mL) was added and the resulting solution was charged to a 500-mL, 3-necked round-bottomed flask containing DABCO (19.4 g) in DMF (50 mL) heated to 70° C. After the addition, residual material was rinsed into the reaction flask with additional DMF (75 mL). The reaction was heated to 70° C. for 2 hours then cooled to room temperature and poured onto water (200 mL). Methylene chloride (200 mL) was added and the biphase was separated. The aqueous phase was extracted with CH₂Cl₂ (100 mL). The combined organic layers were washed with 5% H₂SO₄ (200 mL) then water (200 mL). The organic layer was dried (MgSO₄), filtered and concentrated to afford an orange oil. Methanol (75 mL) was added to the oil and the mixture was heated to dissolve all solids and oils. The product crystallized and was isolated by filtration at 50C to afford 37.2 g of yellow solid (75% assay adjusted molar yield).

### Example 8C

### Scheme I, Step 4: Preparation of (17α)-17-hydroxy-3-oxo-pregna-4,6,9(11)-triene-21-carboxylic Acid γ-lactone (i.e., Δ⁹⁽¹¹⁾-canrenone)

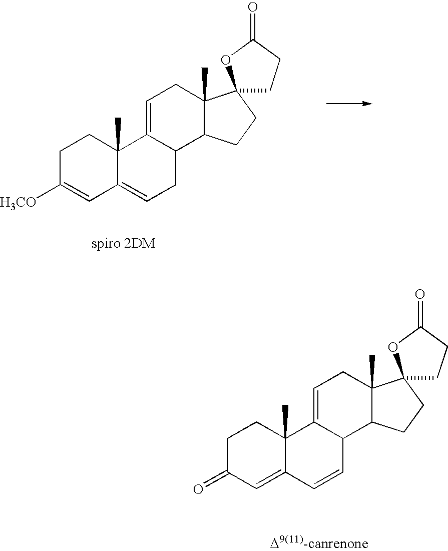

Enol ether substrate (5.0 g), acetone (20 mL) and water (5 mL) were charged to a 50 mL, 3-necked round-bottom flask equipped with a magnetic stirrer. The resulting mixture was cooled to −4° C. and 1,3-dibromo-5,5-dimethylhydantoin (2.2 g) was added in a single charge while maintaining the temperature below 10° C. The reaction was monitored by LC for completion. After completion, the reaction was quenched with ethyl vinyl ether (0.25 mL). The reaction was poured onto NaHCO₃ (10 mL of ½ sat. aq. solution) and ethyl acetate (15 mL) was added. The biphase was separated and the aqueous layer was extracted with ethyl acetate (10 mL). The organic phases were combined and washed twice with water (20 mL each wash). The solution was concentrated to approximately 10 g. DMF (2 mL) was added and the resulting solution was charged to a 50-mL, 3-necked round-bottomed flask containing Li₂CO₃/LiBr (1.3 g each) in DMF (5 mL) heated to 70° C. After the addition, residual material was rinsed into the reaction flask with additional DMF (8 mL). The reaction was heated to 70° C. for 2 hours then cooled to room temperature and poured onto water (25 mL). Methylene chloride (25 mL) was added and the biphase was separated. The aqueous phase was extracted with CH₂Cl₂ (10 mL). The combined organic layers were washed three times with water (25 mL each wash). The organic layer was dried (MgSO₄), filtered and concentrated to afford a yellow oil. Methanol (75 mL) was added to the oil and the mixture was heated to dissolve all solids and oils. The product crystallized and was isolated by filtration at 5° C. to afford 4.0 g of yellow solid (83% molar yield unadjusted for assay).

### Example 9

### Scheme IV, Step 2: Oxidation of Ethynyl 2DM

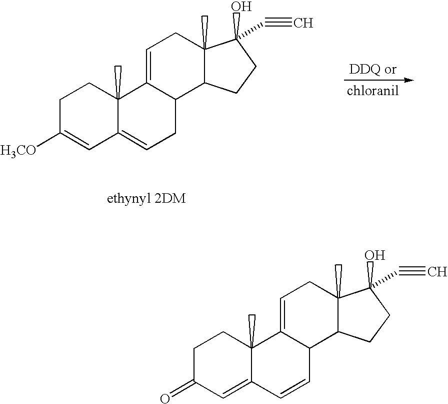

17-ethynyl 2DM (30.00 g) was dissolved in acetone (309 mL) and water (17.1 mL) and chilled to −15° C. while stirring under nitrogen. DDQ (22.42 g) was added while maintaining the temperature below −10° C. The mixture was stirred for 15 min after addition was complete. The reaction was then quenched by slowly adding saturated NaHSO₃ (32.2 mL) with stirring for 30 minutes before concentrating the product mixture. The product mixture was filtered with methylene chloride (350 mL) to recover a solid product which was further washed with methylene chloride. The filtrate was then combined with the washings and extracted three times with water (150 mL, pH 8, Na₂CO₃) followed by an additional extraction with brine (150 mL). The organic layer was dried with Na₂SO₄ and filtered over cartridge grade Magnesol (30 g). After concentration, 27.55 g (96.6% of theory) of pale yellow crystal product were obtained.

### Example 10

### Scheme II, Step 3: 6,7-oxidation of Vinyl 2DM

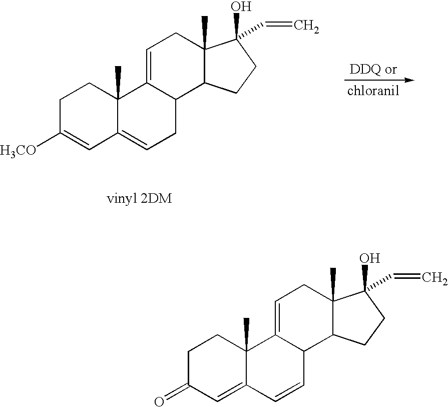

A solution of vinyl 2DM (25.0 g), water (10 mL) and methanol (200 mL) was added to chloranil (19.9 g). The solution was stirred under nitrogen at 42° C. for 1 hour. After cooling the mixture to room temperature, 10% aqueous Na₂S₂O₅ (7.3 mL) was added at a slow rate and stirred for another 20 minutes. Evaporation yielded a solid comprising a crude product. Methylene chloride (80 mL) was added to the crude product and the mixture was chilled to −10° C. before filtering. The filtered solid was washed twice with methylene chloride (20 mL each wash). The filtrate was concentrated and washed to 25 ml and add pulverized KOH (1.65 g). Stir at room temperature for 1.5 hours, filter and wash cake two times with methylene chloride (20 mL). Extract filtrate and washes three times with water and one time with brine. Concentration of the organic phase yielded a pale yellow crystalline product (22.1 g, 92.4%).

### Example 11

### Furylation of (17α)-17-hydroxy-3-oxo-pregna-4,6,9(11)-triene-21-carboxylic Acid γ-lactone (i.e., Δ⁹⁽¹¹⁾-canrenone).

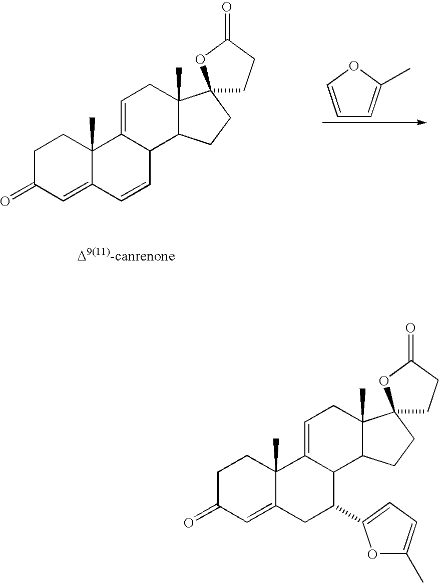

### Example 12

### Scheme VI, Step 3: Furylation

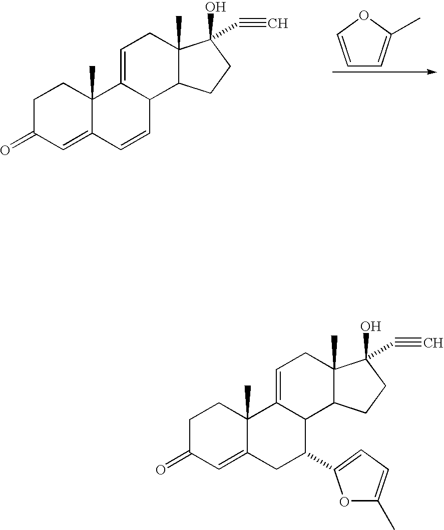

Boron trifluoride etherate (15.0 mL) was added over 30 minutes to a mixture of 3-keto-17-ethynyl substrate (24.43 g), ethanol (6.24 g), 2-methylfuran (14.82 g), and dry acetonitrile (140 mL) at a temperature of −9° C. The suspension was stirred for 16.5 hours at a temperature of −10° C. The resulting red-orange homogeneous solution was quenched by the addition of triethylamine (21 mL) and concentrated to 56 g by rotary evaporation. The residue was partitioned between of aqueous NaOH (ca. 7%, 150 mL) and toluene (150 mL). The aqueous phase was re-extracted with toluene (50 mL) and the combined organic phases were washed with 3 N HCl (100 mL) and brine (50 mL) and dried b (Na₂SO₄). Concentration to about 50 g followed by addition of MTBE (50 mL) and hexanes (50 mL, portion-wise) afforded 23.33 g of light yellow crystals after filtration, washing with 2:1 hexanes/MTBE, and drying in vacuo overnight at 60° C. Analysis by ¹H NMR and HPLC (uncorrected area) was consistent about 95.5 wt % purity (ca. 4.5 wt % total of toluene and MTBE). Concentration of the filtrates to an orange oil and addition of diethyl ether afforded, after drying at 60° C. under vacuum, an additional 1.41 g of product as a white crystalline powder containing about 4 wt % Et₂O. 23.6 g (76.4% of theory) of product was obtained.

### Example 13

### Furylation

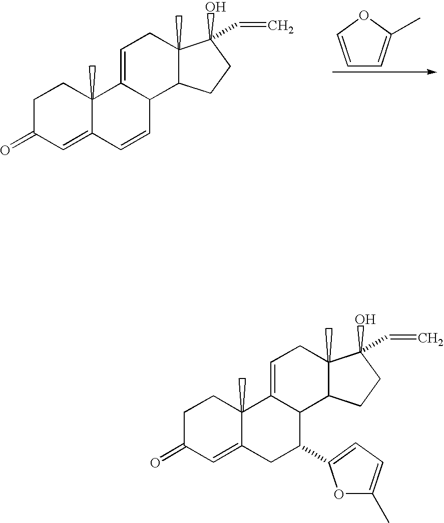

Boron trifluoride etherate (0.59 mL, 4.7 mmol) was added to a −10° C. solution of 3-keto-17-vinyl substrate (1.00 g), 2-methylfuran (0.535 g), and ethanol (0.245 g) in acetonitrile (10 mL). After stirring 19 h at −10° C., the red reaction solution was quenched by the addition of triethylamine (0.59 g, 5.8 mmol). The mixture was partitioned between about 75 mL of dichloromethane and 7 mL of water. The phases were separated and the organic phase was washed with aqueous saturated NaCl. The resulting organic phase was dried over sodium sulfate and evaporated under reduced pressure. Addition of propyl acetate to the resulting residue afforded a white precipitate. The product mixture was filtered, evaporated and diethyl ether was added to give 0.38 g of a yellow crystalline product.

### Example 14

A reactor was charged with crude compound XXXI (1628 g) and methylene chloride (6890 mL). The mixture was stirred to dissolve solids, then dipotassium phosphate (111.5 g) and trichloroacetamide (1039 g) were charged through the hatch. The temperature and agitation were adjusted to 25° C. and 320 RPM, respectively. The mixture was stirred for 90 minutes; then 30% hydrogen peroxide (1452 g) was added over a 10-15 minute period. Stirring was continued at 29-31° C. until less than 4% of the initial charge of compound XXXI remained as determined by periodic HPLC evaluation. This required about 8 hours. At the end of the reaction, water (2400 mL) was added and the methylene chloride portion separated. The methylene chloride layer was washed with a solution of sodium sulfate (72.6 g) in water (1140 mL). After a negative test for peroxide with potassium iodide paper, the methylene chloride fraction was stirred with a caustic solution prepared from 50% sodium hydroxide (256 g) diluted in water (2570 mL) for about 45 minutes in order to remove unreacted trichloroacetamide. The methylene chloride fraction was washed sequentially with of water (2700 mL), then with a solution of sodium bisulfite (190 g ) in water (3060 mL).

The methylene chloride solution of eplerenone was distilled at atmospheric pressure to a final volume of approximately 2500 mL. Methyl ethyl ketone (5000 mL) was charged. The mixture was placed under vacuum distillation and solvent removed to a final volume of approximately 2500 mL. Ethanol (18.0 L) was charged and approximately 3500 mL was removed via atmospheric distillation. The mixture was cooled to 20° C. over a 3-hour period, and then stirred for 4 hours. The solid was collected on a filter and washed twice with 1170 mL of ethanol each time. The solid was dried on the filter under nitrogen for at least 30 minutes. Finally, the solid was dried in a vacuum oven at 75° C. to <5.0% LOD. Thus, 1100 g of the semipure eplerenone was obtained.

Recrystallization of semipure eplerenone from 8-volumes of methyl ethyl ketone (based on contained) provides pure eplerenone with a recovery of about 82%.

### Example 15

The compound of Formula XXXI (160 g crude) was combined with trichloroacetamide (96.1 g), dipotassium phosphate (6.9 g) and methylene chloride (1004 mL or 6.4 ml/g).

Water (25.6 mL) was added to the methylene chloride mixture. The quantity was adjusted to accommodate the concentration of hydrogen peroxide introduced in the following operation. In this case the water was sufficient to dilute the concentration of the subsequently added aqueous hydrogen peroxide (35 wt. %) to a desired level of 30 wt. %.

The mixture of water, steroid substrate, trichloroacetamide and dipotassium phosphate was stirred at 400 RPM and adjusted to 25° C. over a 30 to 45 minute period with a heating mantel connected to a temperature controller.

Thereafter, 35 wt. % hydrogen peroxide (138.4 mL) was added in less than 5 minutes. Although this example utilized 35% hydrogen peroxide, higher concentrations, e.g., 50 wt. %, can be used. As noted, the introduction of aqueous hydrogen peroxide having a strength greater than is desired for the reaction necessitates adding water, typically in the previous step, in order to maintain the desired concentration for the start of the reaction.

The temperature was maintained at 28 to 31° C. throughout the reaction.

The organic portion of the reaction mass was periodically sampled in order to monitor the conversion via HPLC evaluation at 240 nm. A plot of the rate of disappearance of compound XXXI vs. time gave a straight line trend with R²=0.996. The trend predicted a 98% conversion at 712 minutes. The reaction was targeted for a 95 to 98% conversion. Although the reaction was monitored at 240 nm not all of the impurities were observed at this wavelength. In order to get a true profile of the reaction and impurities the assay was re-run at 210 nm.

Water (392 mL) was added to the mixture after 660 minutes (97.7% conversion). In the preparation of this example, the total amount of water was chosen so as to equal the volume of other water charges later in the workup. Addition of water reduced the strength of the peroxide and diminished reactivity towards the steroid components. However, the potential for the generation of low levels of oxygen was still present. The layers were allowed to separate and the lower methylene chloride layer removed (aqueous pH=6.5-7.0). Typically the hydrogen peroxide assayed at about 5 to 6% by weight. This level of concentration correlated with the consumption of 1.5 moles peroxide per mole of compound XXXI converted and a 30% starting concentration.

In a preferred mode of operation, the waste peroxide solution is disposed of via a sulfite quench. This operation is very exothermic and is preferably carried out with slow, controlled combination of the components (either forward or reverse quench modes can be used) in order to control the exotherm. The hydrogen peroxide is reduced to water while the sulfite is oxidized to sulfate during this procedure. After the sulfite quench, the quenched aqueous phase is subjected to a stream stripping operation in order to remove entrained methylene chloride. Prior to steam stripping, the aqueous phase is heated to decarboxylate the trichloroacetate salt that is produced as a by-product arising from conversion of the trichloroacetamide during the course of the epoxidation reaction. Decarboxylation prior to steam stripping prevents the trichloroacetate from reacting with methylene chloride during the stripping operation, which can otherwise result in the formation of chloroform. Decarboxylation can be effected, for example, by heating the aqueous phase at 100° C. for a time sufficient to substantially eliminate the trichoroacetate salt.

The organic phase of the reaction mixture, comprising a methylene chloride solution of eplerenone, was washed for about 15 minutes at 25° C. with an aqueous solution containing Na₂SO₃ (7.4 g) and water (122.4 mL) (pH 7-8). A negative starch iodide test (no purple color with KI paper) was observed in the organic phase at the end of the stir period. If a positive test were observed, the treatment would be repeated.

The methylene chloride fraction was washed with a dilute aqueous sodium hydroxide solution prepared from pellets (7.88 g) and water (392 mL). The mixture was stirred for 35 minutes at 25° C. and then the layers separated (aqueous pH=13). With this short contact time the trichloroacetamide is not completely hydrolyzed but is removed as the salt. In this regard, at least 2 hours is typically required to hydrolyze the trichloroacetamide to the corresponding acid salt, with release of ammonia.

The methylene chloride portion was further washed with water (392 mL). This was intended as a backup wash in case the basic interface was missed. Since the trichloroacetamide is not completely hydrolyzed during the 30-minute contact time, there is a potential for partitioning back into the organic phase once the pH is adjusted (aqueous pH=10).

The methylene chloride portion was washed with a solution of concentrated hydrochloric acid (4.1 mL) in water (352 mL) (pH 1) for about 45 minutes. At the end of this time the pH was adjusted toward neutral with the addition of a solution prepared from sodium sulfite (12.4 g) and water (40 mL ) (pH 6-7).

The methylene chloride solution was concentrated via atmospheric distillation to approximate a vessel minimum stir volume (˜240 mL). About 1024 mL of methylene chloride distillate was collected. Because the preparation of this example was a “virgin run,” i.e., there was no recrystallization mother liquor available for recycle, fresh MEK (1000 mL) was added to the methylene chloride solution of eplerenone, in a proportion (1546 mL in this case) intended to mimic the recycle of mother liquor. Again, the solvent was removed via atmospheric distillation to approximate a minimum stir volume (˜240 mL). Alternatively, these distillations could have been done under vacuum.

Ethanol (2440 mL) was added to the residue. The ethanol charge correlated with 15 mL/g of estimated contained eplerenone for a crude product combined with a typical volume of MEK recrystallization mother liquor (162.7 g). No distinction was made for a virgin batch (144.8 g). Consequently, the virgin run in a campaign as operated at slightly higher volume ratios than runs that contained MEK ML for recovery.

Ethanol was distilled from the slurry (a homogeneous solution was not obtained in this treatment) at atmospheric pressure until 488 mL was removed. The quantity of ethanol removed adjusted the isolation ratio to 12 volumes (not counting the minimum stir volume of about 1.5 mL/g) times the estimated quantity of compound eplerenone contained in the crude product. Since no distinction was made for a virgin run, the isolation volume for this run was slightly inflated. The final mixture was maintained at atmospheric reflux for about one hour.

The temperature of the mixture in the distillation pot was lowered to 15° C. and, after stirring for 4 hours at this temperature, the solid was filtered. The transfer was completed with an ethanol rinse. In general, a 1-2 volume quantity based on contained eplerenone (155 to 310 mL) was utilized in production runs.

The solid was dried in a vacuum oven at 45° C. and semipure material (150.8 g) with an 89.2% assay was obtained as the output of a virgin run (154.6 g assay adjusted is the expected output for runs that include an MEK recrystallization mother liquor recovery). Generally, 94-95% of the available eplerenone was recovered after this first stage upgrade of crude product. The designated level of drying allowed isolation of the semipure eplerenone as the ethanol solvate. In this regard, the solvate does not easily release ethanol until the temperature reaches about 90° C. The solvate is preferred for further processing since the desolvated material tends to clump upon mixing with MEK in the next operation.

The solid is combined with of 2-butanone (MEK) (2164 mL). This quantity of MEK corresponds with a volume ratio of 14 mL/g vs. the estimate of contained eplerenone (includes MEK mother liquor portion).

A hot filtration of the eplerenone in MEK solution is preferably carried out prior to recrystallization, but was not employed in the laboratory run. The filtration is normally followed with a rinse quantity correlating with 2 volumes of MEK based on contained eplerenone, e.g., 310 mL. This gives a total MEK volume of 2474 mL that correlates with 16 mL/g. The hot filtration should not be operated below a ratio of 12 mL/g since this is the estimated saturation level for eplerenone in MEK at 80° C.

MEK was distilled from the solution at atmospheric pressure until 1237 mL was removed. This correlated with 8 volumes and adjusted the crystallization ratio to a volume of 8 mL/g vs. the quantity of eplerenone estimated in the semipure product. The actual volume remaining in the reactor is 8 mL/g plus the solid void estimated at 1-1.5 volumes for a total isolation target volume of 9-9.5 mL/g.

The solution (the mixture is supersaturated at this point and nucleation may occur before the cool down starts) is cooled according to the following schedule. This stepwise strategy has consistently generated polymorph II.

Cool to 65° C. and hold for 1 hour.

Cool to 50° C. and hold for 1.5 hours.

Cool to 35° C. and hold for 1 hour.

Cool to 15° C. and hold for 1 hour.

Then the solid is filtered and rinsed with MEK (310 mL).

The solid was initially dried on the filter at 25° C. overnight. Then drying and desolvation were completed in a vacuum oven at 80-90° C. for ca. 4 hours. The expected dry solid weight is 119.7 g for a virgin run and 134.5 g for a run with MEK mother liquor inclusion. The LOD of the final product should be <0.1%. The filtrate (1546 mL) contained ca. 17.9 g of eplerenone. This correlated with 11.5 wt. % of adjusted input of compound XXI. The mother liquor was saved for recovery via combination with a subsequent ethanol treatment. Data have indicated that the product eplerenone was stable up to 63 days in MEK at 40° C.

The overall assay adjusted weight yield was 76.9%. This overall yield is composed of 93, 95 and 87 assay adjusted weight % yields for the reaction, ethanol upgrade and MEK recrystallization, respectively. There is a potential 1 to 2% yield loss related to the NaOH treatment and associated aqueous washes. Inclusion of the MEK mother liquor in subsequent runs is expected to increase the overall yield by 9.5% (11.5×0.95×0.87) for an adjusted total of 86.4%.

The MEK mother liquor can be combined with a methylene chloride solution from the next epoxidation reaction and the procedure, as described above, repeated.

In view of the above, it will be seen that the several objects of the invention are achieved and other advantageous results attained. As various changes can be made in the above processes and compositions without departing from the scope of the invention, it is intended that all matter contained in the above description shall be interpreted as illustrative and not in a limiting sense.

1-50. (canceled)

51. A process for the preparation of a compound corresponding to the Formula 2503: 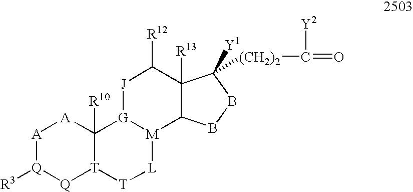
wherein
Y¹ and Y² together represent the oxygen bridge —O— or Y¹ represents hydroxy and Y² represents hydroxy alkoxy or O^{—};
R³ is selected from the group consisting of hydrogen, hydroxy, alkoxy, hydroxyalkyl, alkoxyalkyl and hydroxycarbonyl, dihydrocarbylamino, di(substituted hydrocarbyl)amino and N-heterocyclyl;
R¹⁰, R¹² and R¹³ are independently selected from the group consisting of hydrogen, halo, hydroxy, alkyl, alkoxy, hydroxyalkyl, alkoxyalkyl, hydroxycarbonyl, cyano and aryloxy;
-A-A- represents the group —CHR¹—CHR²— or —CR¹═CR²—;
where R¹ and R² are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano and aryloxy or R¹ and R² together with the carbons of the steroid nucleus to which they are attached form a (saturated) cycloalkylene group;
—B—B— represents the group —CHR¹⁵CHR¹⁶—, —CR¹⁵═CR¹⁶— or an α- or β-oriented group: 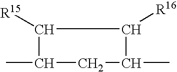
where R¹⁵ and R¹⁶ are independently selected from the group consisting of hydrogen, halo, alkyl, alkoxy, acyl, hydroxyalkyl, alkoxyalkyl, hydroxycarbonyl, alkoxycarbonyl, acyloxyalkyl, cyano and aryloxy;
or R¹⁵ and R¹⁶, together with the C-15 and C-16 carbons of the steroid nucleus to which they are respectively attached, form a (saturated) cycloalkylene group;
-G-J- represents the group 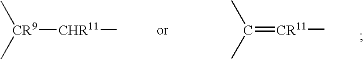
where R⁹ and R¹¹ are independently selected from the group consisting of hydrogen, hydroxy, protected hydroxy, halo, alkyl, alkoxy, acyl, hydroxyalkyl, alkoxyalkyl, hydroxycarbonyl, alkoxycarbonyl, acyloxyalkyl, cyano and aryloxy;
-Q-Q- represents the group 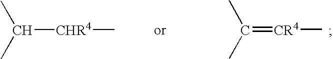
where R⁴ is selected from the group consisting of hydrogen, halo, alkyl, alkoxy, acyl, hydroxyalkyl, alkoxyalkyl, hydroxycarbonyl, alkoxycarbonyl, acyloxyalkyl, cyano and aryloxy; or 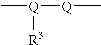
together represent the group 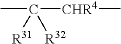
where R³¹ and R³² are independently selected from the group consisting of hydroxy and alkoxy, or R³¹, R³² and the C-3 carbon of the steroid nucleus to which they are attached form the group 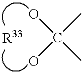
where R³³ is alkylene.
-T-T- represents the group 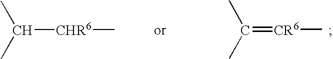
where R⁶ is selected from the group consisting of hydrogen, halo, alkyl, alkoxy, acyl, hydroxyalkyl, alkoxyalkyl, hydroxycarbonyl, alkoxycarbonyl, acyloxyalkyl, cyano and aryloxy; and
-L-M- represents the group 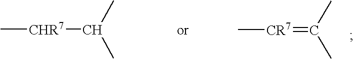
where R⁷ is selected from the group consisting of hydrogen, halo, hydroxy, protected hydroxy, alkyl, cycloalkyl, alkoxy, acyl, hydroxyalkyl, alkoxyalkyl, hydroxycarbonyl, alkoxycarbonyl, acyloxyalkyl, cyano, aryloxy, heteroaryl, heterocyclyl, acetylthio, furyl and substituted furyl;
or R⁶ and R⁷, together with the C-6 and C-7 carbons of the steroid nucleus to which they are respectively attached, form a (saturated) cycloalkylene group;
or R⁵ and R⁷, together with the C-5, C-6 and C-7 carbons of the steroid nucleus form a pentacyclic ring fused to the steroid nucleus and corresponding to the structure: 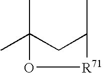
wherein R⁷¹ comprises ═CH(OH), ═CH(OR⁷²) or ═CH═O,
the process comprising:
carbonylating a 17-vinyl-17-hydroxy steroid compound of Formula 2502: 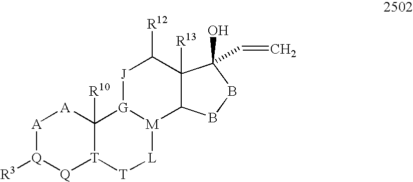
where the substituents R³, R¹⁰, R¹², R¹³, -A-A-, —B—B—, -G-J-, -Q-Q-, -T-T- and -L-M- are as defined in Formula 2503

52. A process as set forth in claim 51, wherein the process further comprises:
preparing the compound of Formula 2502 by reducing the 17-ethynyl group of a compound of Formula 2501 to a 17-vinyl group, said compound of Formula 2501 having the structure: 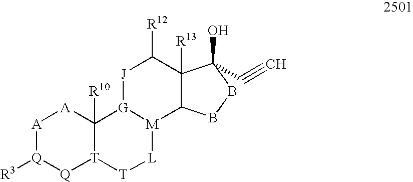
where the substituents R³, R¹⁰, R¹², R¹³, -A-A-, —B—B—, -G-J-, -Q-Q-, -T-T- and -L-M- are as defined above in Formula 2503.

53. A process as set forth in claim 51 wherein said compound of Formula 2501 is contacted with a source of hydrogen in a hydrogenation reaction zone, thereby reducing the 17-ethynyl group and yielding an intermediate comprising the 17-vinyl compound corresponding to Formula 2502; and
the derivative of Formula 2502 is contacted with a source of carbon monoxide and a carbonylation catalyst in a carbonylation reaction zone to yield the product of Formula 2503.

54. A process as set forth in claim 52 wherein said compound of Formula 2501 is contacted with a source of hydrogen in the presence of a catalyst.

55. A process as set forth in claim 53 wherein said intermediate derivative of Formula 2502 is removed from said hydrogenation reaction zone and transferred to said carbonylation reaction zone.

56. A process as set forth in claim 53 wherein said compound of Formula 2501 is simultaneously contacted with a source of hydrogen, a source of carbon monoxide and a catalyst system effective for reducing the 17-ethynyl group of the compound of Formula 2501 to a 17-vinyl group and for carbonylating the resulting derivative of Formula 2502 in situ to convert the 17-hydroxy-17-vinyl structure thereof to a 17-spirobutyrolactone structure.

57. A process as set forth in claim 53 wherein the hydrogenation reaction is conducted in the presence of an alkene or cycloalkene.

58. A process as set forth in claim 57 wherein said alkene or cycloalkene function as a solvent for said compound of Formula 2501.

59. A process as set forth in claim 58 wherein said alkene or cycloalkene further functions as a solvent for said compound of Formula 2502.

60. A process as set forth in claim 57 wherein said compound of Formula 2501 is simultaneously contacted with a source of hydrogen, a source of carbon monoxide and a catalyst system effective for reducing the 17-ethynyl group of the compound of Formula 2501 to a 17-vinyl group and for carbonylating the resulting derivative of Formula 2502 in situ to convert the 17-hydroxy-17-vinyl structure thereof to a 17-spirobutyrolactone structure; and said alkene or cycloalkene functions as a solvent for the product compound of Formula 2503.

61. A process as set forth in claim 53 wherein the 17-ethynyl group is reduced to a 17-vinyl group in a hydrogenation reaction zone in the presence of a sacrificial hydrogenation target, thereby inhibiting the hydrogenation of the 17-vinyl group to a 17-ethyl group.

62. A process as set forth in claim 53 wherein reduction of said 17-ethynyl group comprises contacting said compound of Formula 2501 with a source of hydrogen in the presence of a noble metal catalyst.

63. A process as set forth in claim 62 wherein the catalyst comprises Pd on a calcium carbonate support.

64. A process as set forth in claim 52 wherein hydrogenation of said 17-ethynyl group is conducted at a temperature of from about 0° to about 100° C.

65. A process as set forth in claim 64 wherein said hydrogenation is conducted a temperature of from about 25° to about 75° C.

66. A process as set forth in claims 52 wherein hydrogenation of said 17-ethynyl group is conducted at a pressure of from about 0 to about 100 psig.

67. A process as set forth in claim 66 wherein said hydrogenation is conducted at a pressure of from about 25 to about 50 psig.

68. A process as set forth in claim 67 wherein the hydrogenation reaction is conducted in a solvent comprising a lower alcohol selected from the group consisting of methanol, ethanol and isopropanol.

69. A process as set forth in claim 51 in which said carbonylation catalyst is formed by contacting a source of a metal with a source of carbon monoxide.

70. A process as set forth in claim 69 wherein the carbonylation catalyst is formed by contacting the source of metal with a source of carbon monoxide in the presence of a ligand.

71. A process as set forth in claim 69 wherein the carbonylation catalyst is formed in the presence of a reducing agent.

72. A process as set forth in claim 69 wherein the carbonylation catalyst is formed in situ in the carbonylation reaction medium.

73. A process as set forth in claim 69 wherein the carbonylation catalyst comprises a metal selected from the group consisting of Co, Ni, Fe, Pt, Pd, Ru, Rh, Ir and mixtures thereof.

74. A process as set forth in claim 71 wherein said carbonylation catalyst is formed by contacting a source of Pd, a ligand and a reducing agent.

75. A process as set forth in claim 71 wherein the ligand comprises phosphorus.

76. A process as set forth in claim 70 wherein the reducing agent comprises an active hydrogen source.

77. A process as set forth in claim 76 wherein the reducing agent is selected from the group consisting of hydrogen, formic acid, borohydrides and oxalic acid.

78. A process as set forth in claim 77 wherein the reducing agent comprises formic acid.

79. A process as set forth in claim 51 wherein said intermediate of Formula 2502 is contacted with carbon monoxide at a temperature of from about 80° to about 150° C.

80. A process as set forth in claim 79 wherein said intermediate of Formula 2502 is contacted with carbon monoxide at a temperature of from about 100° to about 105° C.

81. A process as set forth in claim 52 comprising:
contacting said compound of Formula 2501 with a source of hydrogen and a hydrogenation catalyst in a liquid reaction medium comprising a solvent, thereby producing a hydrogenation reaction mixture comprising a hydrogenation reaction solution comprising said intermediate of Formula 2502 in said solvent; and
mixing said hydrogenation reaction solution or a concentrate thereof with water to produce a liquid crystallization medium in which the solubility of said compound of Formula 2502 is lower than the solubility thereof in said solvent alone; and
crystallizing said compound of Formula 2502.

82. A process as set forth in claim 81 wherein said hydrogenation reaction solution or concentrate thereof is filtered for removal of catalyst prior to mixing thereof with water.

83. A process as set forth in claim 51 in which said compound of Formula 2502 is contacted with a source of carbon monoxide and a carbonylation catalyst in a liquid reaction medium comprising a solvent for the compound of Formula 2502, thereby producing a carbonylation reaction mixture comprising a carbonylation reaction solution comprising said compound of Formula 2503.

84. A process as set forth in claim 83 wherein said product of Formula 2503 is recovered from a final crystallization medium comprising said carbonylation reaction solution or derived therefrom.

85. A process as set forth in claim 84 wherein said carbonylation reaction solution is mixed with a solvent that is miscible with the liquid reaction medium but in which the solubility of said compound of Formula 2501 is lower than it is in the liquid reaction medium, resulting in crystallization of said compound of Formula 2501 from the resulting crystallization medium.

86. A process as set forth in claim 84 wherein, prior to crystallization of said compound of Formula 2501, said carbonylation reaction solution or a concentrate thereof is filtered for removal of any solids contained therein.

87. A process as set forth in claim 86 wherein solids removed by filtration from said carbonylation reaction solution or concentrate thereof are washed with a solvent which is combined with the filtrate prior to crystallization.

88. A compound of Formula XXII: 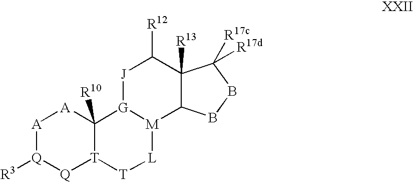
wherein:
R³ is selected from the group consisting of hydrogen, hydroxy, alkoxy, hydroxyalkyl, alkoxyalkyl and hydroxycarbonyl;
R¹⁰, R¹², and R¹³ are independently selected from the group consisting of hydrogen, halo, hydroxy, lower alkyl, lower alkoxy, hydroxyalkyl, alkoxyalkyl, hydroxycarbonyl, cyano, and aryloxy;
R^{17c} is selected from the group consisting of hydroxy and protected hydroxy; and
R^{17d} is alkenyl;
-A-A- represents the group —CHR¹—CHR²— or —CR¹═CR²—;
where R¹ and R² are independently selected from the group consisting of hydrogen, halo, hydroxy, alkyl, alkoxy, acyl, hydroxyalkyl, alkoxyalkyl, hydroxycarbonyl, alkoxycarbonyl, cyano, and aryloxy, or R¹ and R² together with the carbons of the steroid backbone to which they are attached form a cycloalkyl group;
—B—B— represents the group —CHR¹⁵—CHR¹⁶— or an α- or β-oriented group: 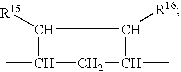
where R¹⁵ and R¹⁶ are independently selected from the group consisting of hydrogen, halo, alkyl, alkoxy, acyl, hydroxyalkyl, alkoxyalkyl, hydroxycarbonyl, alkoxycarbonyl, acyloxyalkyl, cyano, and aryloxy;
-G-J- represents the group 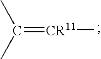
where R¹¹ is selected from the group consisting of hydrogen, halo, alkyl, alkoxy, acyl, hydroxyalkyl, alkoxyalkyl, hydroxycarbonyl, alkoxycarbonyl, acyloxyalkyl, cyano and aryloxy;
-Q-Q- represents the group 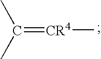
where R⁴ is selected from the group consisting of hydrogen, halo, alkyl, alkoxy, acyl, hydroxyalkyl, alkoxyalkyl, hydroxycarbonyl, alkoxycarbonyl, acyloxyalkyl, cyano and aryloxy;
-T-T- represents the group 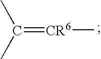
where R⁶ is selected from the group consisting of hydrogen, halo, alkyl, alkoxy, acyl, hydroxyalkyl, alkoxyalkyl, hydroxycarbonyl, alkoxycarbonyl, acyloxyalkyl, cyano and aryloxy; and
-L-M- represents the group 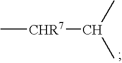
where R⁷ is selected from the group consisting of hydrogen, halo, alkyl, cycloalkyl, alkoxy, acyl, hydroxyalkyl, alkoxyalkyl, hydroxycarbonyl, alkoxycarbonyl, acyloxyalkyl, cyano, aryloxy, acetylthio, furyl and substituted furyl.

89. A compound according to claim 88 corresponding to Formula XXIIA: 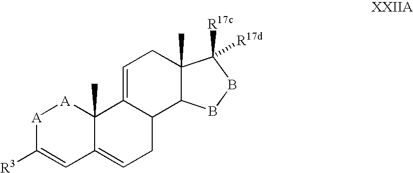
wherein
-A-A- represents the group —CH₂—CH₂— or —CH═CH—;
R³ is lower alkoxy;
—B—B— represents the group —CH₂—CH₂— or an α- or β-oriented group: 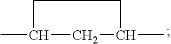
R^{17a} is hydroxy or protected hydroxy; and
R^{17b} is alkenyl.

90. A compound according to claim 89 corresponding to Formula A:

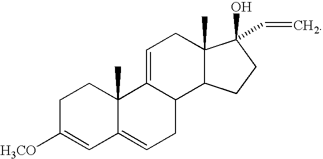

91-193. (canceled)

194. A process for the preparation of a compound corresponding to Formula 1001: 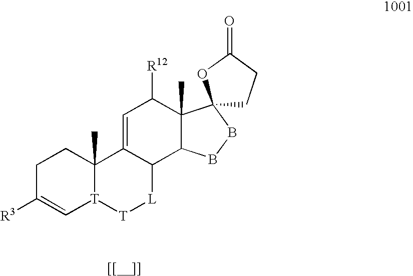
the process comprising carbonylating a 17-vinyl-17-hydroxy steroid compound of Formula 1000: 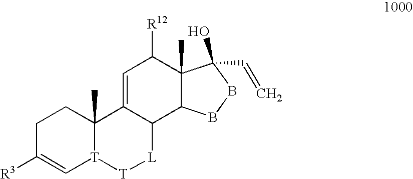
wherein
R³ is selected from the group consisting of hydrogen, hydroxy, alkoxy, hydroxyalkyl, alkoxyalkyl and hydroxycarbonyl, dihydrocarbylamino, di(substituted hydrocarbyl)amino and N-heterocyclyl;
R¹² is selected from the group consisting of hydrogen, halo, hydroxy, alkyl, alkoxy, hydroxyalkyl, alkoxyalkyl, hydroxycarbonyl, cyano and aryloxy;
-T-T- represents the group 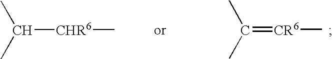
where R⁶ is hydrogen;
-T-L- represents the group —CHR⁶—CHR⁷—; where R⁶ is hydrogen;
where R⁷ is selected from the group consisting of hydrogen and acetylthio;
or R⁶ and R⁷, together with the C-6 and C-7 carbons of the steroid nucleus to which they are respectively attached, form a (saturated) cycloalkylene group;
—B—B— represents the group —CHR¹⁵—CHR¹⁶—; where R¹⁵ and R¹⁶ are hydrogen;
or R¹⁵ and R¹⁶, together with the C-15 and C-16 carbons of the steroid nucleus to which they are respectively attached, form a (saturated) cycloalkylene group.

195. The process of claim 194 further comprising the preparation of a compound corresponding to Formula 1002: 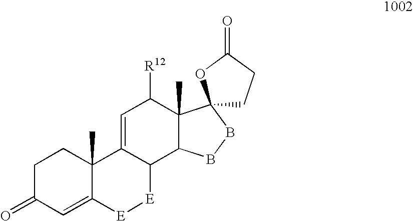
where
R¹² is selected from the group consisting of hydrogen, halo, hydroxy, alkyl, alkoxy, hydroxyalkyl, alkoxyalkyl, hydroxycarbonyl, cyano and aryloxy;
-E-E- represents the group —CHR⁶—CHR⁷—; where R⁶ is hydrogen;
where R⁷ is selected from the group consisting of hydrogen and acetylthio;
or R⁶ and R⁷, together with the C-6 and C-7 carbons of the steroid nucleus to which they are respectively attached, form a (saturated) cycloalkylene group;
—B—B— represents the group —CHR¹⁵—CHR¹⁶—; where R¹⁵ and R¹⁶ are hydrogen;
or R¹⁵ and R¹⁶, together with the C-15 and C-16 carbons of the steroid nucleus to which they are respectively attached, form a (saturated) cycloalkylene group;
the process comprising oxidizing a steroid compound of Formula 1001.

196. The process of claim 195 further comprising the preparation of a compound of Formula 1003: 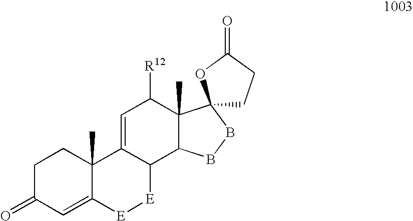
where
R⁷ is selected from the group consisting of furyl, alkylfuryl, thienyl, and alkylthienyl;
the process comprising contacting a compound of Formula 1002 where R⁷ is hydrogen, with furan, alkylfuran, thiophene or alkylthiophene;
R¹², -E-E- and —B—B— are defined as in Formula 1002 above.

197. The process of claim 196 further comprising preparation of a compound of Formula 1004: 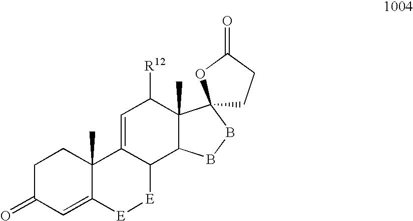
where
R⁷ is —C(O)OH;
the process comprising contacting the compound of formula 1003 where R⁷ is selected from the group consisting of furyl, alkylfuryl, thienyl, and alkylthienyl; with an oxidizing agent and an ozonolysis agent;
R¹², -E-E- and —B—B— are defined as in Formula 1003 above.

198. The process of claim 197 further comprising the preparation of a compound of Formula 1005: 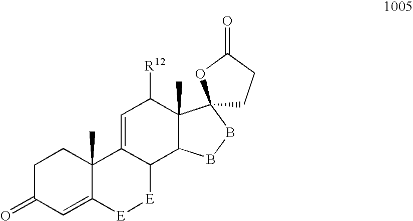
where
R⁷ is —C(O)OR⁷¹;
R⁷¹ is an alkyl group;
the process comprising esterifying a 7-carboxyl steroid compound of Formula 1004 where R⁷ is —C(O)OH;
R¹², -E-E- and —B—B— are defined as in Formula 1004 above.

199. The process of claim 198 further comprising preparation of a compound of Formula 1006 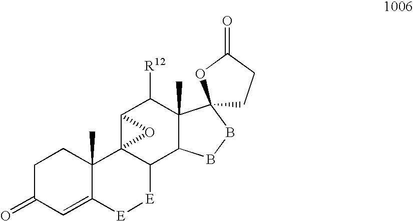
R¹², —B—B— and -E-E are defined as in Formula 1005 above;
the process comprising contacting the compound of Formula 1005 with an epoxidation agent.

200. The process of claim 199 further comprising preparation of a compound of Formula 999: 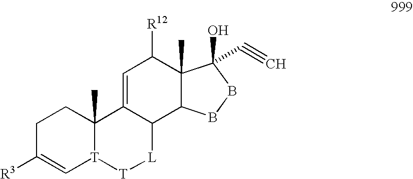
the process comprising ethynylating a 17-keto steroid compound of Formula 1997: 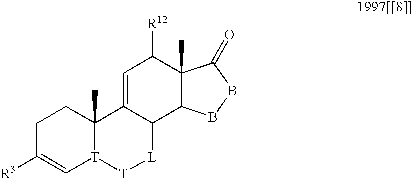
where R³, R¹², -T-T-, -T-L- and —B—B— are as defined in Formula 1001.

201. The process of claim 200 further comprising preparation of a compound of Formula 1000, the process comprising hydrogenating a compound of Formula 999.
